(19) United States
(12) Patent Application Publication
(10) Pub. No.: US 2022/0041988 A1
(43) Pub. Date: Feb. 10, 2022

(54) GENERATION OF FUNCTIONAL AND PATIENT-SPECIFIC THYMIC TISSUE IN VIVO FROM INDUCED PLURIPOTENT STEM CELLS

(71) Applicant: THE REGENTS OF THE UNIVERSITY OF COLORADO, A BODY CORPORATE, Denver, CO (US)

(72) Inventors: Holger A. Russ, Denver, CO (US); Stephan A. Ramos, Aurora, CO (US); Antonio Jimeno, Greenwood Village, CO (US); John Jason Morton, Thornton, CO (US)

(21) Appl. No.: 17/511,228

(22) Filed: Oct. 26, 2021

Related U.S. Application Data
(63) Continuation of application No. PCT/US2020/030130, filed on Apr. 27, 2020.
(60) Provisional application No. 62/839,107, filed on Apr. 26, 2019.

(51) Int. Cl. C12N 5/0789; C12N 5/074; A61K 35/26; A61K 35/17

(52) U.S. Cl. CPC C12N 5/0647; C12N 5/0696; A61K 35/26; A61K 35/17; C12N 2506/11; C12N 2506/45; C12N 2501/115; C12N 2501/41; C12N 2501/415; C12N 2501/16; C12N 2501/155; C12N 2501/385; C12N 2501/15

(57) ABSTRACT

The disclosed technology includes methods, systems, and devices for generating patient-specific functional thymic epithelial progenitor (TEP) cells. In some implementations, a method may include generating iPSCs from HSC; causing differentiation of the iPSC into thymic epithelial progenitor (TEP) cells, generating thymic epithelial cells by transplantation of the TEP cells into a host, wherein the TEP cells may differentiate into mature functional thymic epithelial cells (TECs). In some implementations, a system may include a cell population of patient specific cells, a population of iPSCs, a culture system for differentiating the iPSCs into a population of patient-specific TEP cells for transfer to a host or the patient to allow the TEP cells to differentiate into mature, functional TEC.

### CROSS-REFERENCE TO RELATED APPLICATIONS

This U.S. Continuation application claims the benefit of International Application, PCT/US20/30130 filed on Apr. 27, 2021 which claims the benefit of priority pursuant to 35 U.S.C. § 119(e) to U.S. provisional patent application No. 62/839,107 filed on Apr. 26, 2019. These applications are hereby incorporated by reference in their entirety for all purposes.

### GOVERNMENT LICENSE RIGHTS

This invention was made with government support under grant number CA213102 and CA149456 awarded by National Institutes of Health. The government has certain rights in the invention.

### FIELD

The disclosed processes, methods, and systems are directed to in-vitro generation of human thymic epithelial progenitor cells from induced pluripotent stem cells, that may be further differentiated into patient-specific thymic epithelial cells.

### SEQUENCE LISTING

The instant application contains a Sequence Listing which has been submitted electronically in ASCII format and is hereby incorporated by reference in its entirety. Said ASCII copy, created on Oct. 25, 2021, is named 106549-688764 CU4898H-PCT1_ST25.txt and is 101 Kilobytes in size.

### BACKGROUND

The thymus is a glandular organ that is necessary for the development of the T-cell repertoire. The thymus functions in the positive and negative selection of T-cells for the establishment of an adaptive, self-tolerant immune system. Defects in thymus development and function may result in the onset of immunological deficiencies and autoimmune diseases. Traditionally, studies on thymus development and autoimmunity have been largely restricted to mouse models. This is predominantly due to the lack of availability of developing human thymi, and the lack of a faithful in vitro human thymus model.

Methods are needed to modulate the immune system of autoimmune patients, such as those with Type 1 Diabetes (T1D), to (re-)induce tolerance to self-antigens. Additionally, patients who have undergone lymphopenia from chemotherapy and/or hematopoietic stem cell treatment suffer from slow or incompetent T-cell reconstitution due to the lack of a competent thymic epithelium as an adult. This slow and incompetent T-cell reconstitution may leave patients susceptible to a plethora of infectious diseases, relapse, and graph versus host disease. Furthermore, since the thymus undergoes age related atrophy, aged individuals are less immune competent than younger individuals. Patients undergoing treatment related with solid organ or stem cell derived cell/tissue transplantation are also susceptible to potential immunomodulation, and therefore in need of methods to reduce or abolish the need for immunosuppression post-transplant. Previous studies in mice have shown that providing aged mice with a young thymus may prolong the life of the aged mice and that forcing expression of FOXN1 in the thymi of aged mice may regenerate and restore thymic function. Thus, regeneration of a functional thymus in aged individuals could serve as an attractive therapy to combat the effects of aging in humans, potentially leading to prolonging human life expectancy. However, there are critical gaps in the current understanding of human thymic development and function.

### SUMMARY

The disclosed technology is directed to methods for generating patient-specific thymic epithelial cells (TECs), the method comprising, isolating a cell from the patient, administering one or more factors to the cell to reprogram the cell and create an induced pluripotent stem cell (iPSC), culturing the patient-specific iPSC for 9-14 days in a differentiation media to create a thymic epithelial progenitor (TEP) cell, and transferring at least one TEP into a recipient, and allowing the TEP cell to differentiate into a TEC. In many embodiments, the iPSC may be derived from a hematopoietic stem cell (HSC) or peripheral blood mononuclear cell (PBMC), and the resulting TECs may be mature, functional, patient-specific thymic epithelial cells (TEC). In some embodiments, the method may include a step of contacting a patient-derived T-cell with the TEC to produce a functional T-cell, or a step of contacting a patient-derived T-cell with the TEPs to produce a functional T-cell and or functional TECs. In some embodiments, the mature T-cell expresses one or more of CD69, CD25, CD5, CD7, CD4, CD8, CD3, CD45, RAG1, and RAG2. In many embodiments, the number of peripheral T-cells is greater than in a recipient that did not receive a patient-specific TEP. In many embodiments, the differentiation media comprises one or more pathway activators and/or pathway inhibitors, for example one or more of Activin, WNT, BMP, RA, TGFβ, SHH, and FGFβ, which may be affected by at least one of Activin A, WNT3a, BMP4, SAG, TTNPB, FGF8b, Ly-364947, and Sant1. In some methods, there may be a co-culturing step, wherein the TEP cells with hematopoietic stem and progenitor cells (HPSCs) or HSCs for about 7 days generates TECs. In many of these methods, the TECs may express one or more genetic markers selected from FOXN1, AIRE, CK5, CK8, CXCL12, CCL25, DLL4, TP63, CBX4, and HLA-DR, such as markers typically expressed by cortical TECs and medullary TECs, such as KRT5, KRT8, AIRE, PSMB11, and PRSS16.

Also disclosed are populations of differentiated, mature thymic epithelial cells comprising one or more thymic epithelial cells (TECs) expressing one or more of KRT5, KRT8, AIRE, PSMB11, and PRSS16, wherein the one or more TECs is derived from thymic epithelial progenitor (TEP) cell derived from an induced pluripotent stem cell (iPSC) grown in-vitro in the presence of one or more of Activin A, Wnt3a, TTNPB, BMP4, LY364947, FGF8b, FGF8a, SAG, and SANT-1, and wherein the TEP differentiates into a TEC in-vivo after transplantation into a recipient. In many embodiments, the population of differentiated, mature thymic epithelial cells, the iPSCs may be grown in-vitro for between 12 and 14 days, and may be derived from one or more cells of the recipient. The resulting TECs may express one or more markers selected from FOXN1, AIRE, CK5, CK8, CXCL12, CCL25, DLL4, and HLA-DR.

Also disclosed are systems for generating mature functional thymic epithelial cells comprising a method for inducing a pluripotent stem cell from a cell of a subject, a culture device for growing the induced pluripotent stem cell for 12-14 days in the presence and absence of one or more of Activin A, Wnt3a, TTNPB, BMP4, LY364947, FGF8b, FGF8a, SAG, and SANT-1 to produce a differentiated thymic epithelial progenitor cell, a device for implanting one or more thymic epithelial progenitor cells into a subject.

Also described are uses of populations of cells, as described above and herein, in the preparation of a medicament for the treatment of an immune condition or disorder, wherein the disorder or condition is selected from a non-existent thymus, damaged thymus, dysfunctional thymus, diseased thymus, aged thymus, diabetes Type 1, auto-immune, allorejection, cancer, and combinations thereof.

Also disclosed are methods of treating a subject suffering from or at risk of an immune condition or disorder, comprising, administering to the subject a TEP cell according to any of claims 19-22, or a cell produced by the method of any of claims 1-18; wherein the disorder or condition is selected from a non-existent thymus, damaged thymus, dysfunctional thymus, diseased thymus, aged thymus, Type 1 diabetes, auto-immune, allorejection, cancer, and combinations thereof.

Also disclosed are methods of treating a patient with a thymic disorder comprising administering one or more thymic epithelial progenitor (TEP) cells derived from a patient specific induced pluripotent stem cell (iPSC) grown in-vitro in the presence of one or more of Activin A, Wnt3a, TTNPB, BMP4, LY364947, FGF8b, FGF8a, SAG, and SANT-1, and wherein the TEP differentiates into a thymic epithelial cells (TECs) in-vivo after administration to the patient, wherein the iPSCs are grown in-vitro for between 12 and 14 days, and wherein the mature TECs express one or more markers selected from FOXN1, AIRE, CK5, CK8, CXCL12, CCL25, DLL4, and HLA-DR. In some embodiments, the iPSCs are derived from one or more of the patient's skin, uterine tissue, kidney, liver, muscle, adrenal glands, blood, and or the selected marker is expressed at between 0.5-fold and 1000-fold in mature iPSC-derived TECs relative to the administered TEP cells or the iPSCs.

### BRIEF DESCRIPTION OF THE DRAWING

The patent or application file contains at least one drawing executed in color. Copies of this patent or patent application publication with color drawing(s) will be provided by the Office upon request and payment of the necessary fee.

FIG. 1A is a schematic of direct differentiation approach to generate TEPs from iPSCs. Marker genes for each stage are indicated.

FIG. 1B is a table of differentiation conditions and factors tested. All 6 conditions tested compared to d0 iPSCs and neonatal human thymus samples (Thy).

FIGS. 1C and 1D show data on gene expression analysis for third pharyngeal pouch markers, HOXA3 and EYA1, and TEP marker FOXN1 at day 9 (FIG. 1C) and 14 (FIG. 1D). Values shown are relative quantification (RQ) compared to d0 iPSCs and were normalized to ACTB (n=3, two iPSC lines, n=2 primary human thymi). Data shows the mean with error bars indicating the standard error of the mean (SEM). P-values, as determined by one-way ANOVA with Tukey's multiple comparisons test, using ddCt values: (FIG. 1C) HOXA3: ****=p<0.0001. EYA1: ***=p<0.0007, **=p<0.0016. (FIG. 1D) HOXA3: †=0.034, and †††† signifies significance between primary thymus and all 6 conditions with p=<0.0001.**** signifies significance between d0 iPSC and all 6 samples with p=<0.0001. EYA1: ***=p<0.001, **=p<0.002. FOXN1: p=0.0024.

FIG. 1E are representative micrographs of immunofluorescence staining for FOXA2 (red) and HOXA3 (green) protein expression at day 9 of TEP differentiation using condition 4. Nuclei are counterstained with DAPI. Scale bar=20 μm.

FIG. 1F is a graph of quantification of HOXA3 positive cells at day 9 of TEP differentiations using condition 4. Graphed as mean percent HOXA3 positive cells over total DAPI+cells, with error bars indicating the SEM (n=3, 3 iPSC lines).

FIG. 1G are graphs of gene expression analysis of third pharyngeal pouch markers, HOXA3 and EYA1, and TEP marker FOXN1 at day 14 of TEP differentiations with condition 4 (plus supplements) using multiple iPSC lines (n=3, 3 iPSC lines). Data graphed as the mean relative expression, with the error bars indicating SEM. P-values, as determined by unpaired t-test using ddCt values: HOXA3=0.0003, EYA1=0.0064.

FIG. 2A is a schematic of experimental design for in vivo maturation of iPSC-derived TEPs to TECs.

FIG. 2B shows data from gene expression analysis of iPSC-derived TEPs generated using condition 4 at day 20 (n=2, two iPSC lines), and grafts recovered from nude mice recipients (n=8) 14-17 weeks post transplantation. Graphs of relative quantification (RQ) values are shown. Data shown as the mean with error bars showing the SEM normalized to ACTB and set relative to experimentally matched day 0 iPSCs. P-values, as determined by one-way ANOVA with Tukey's multiple comparisons test, using ddCt values: FOXN1: ****p=<0.0001, ***p=0.0001. KRT5: p=<0.0001. DLL4: p=0.041

FIG. 2C are representative micrograph immunofluorescence images of TEP grafts stained for mTEC and cTEC markers cytokeratin 5 (green) and cytokeratin 8 (red), respectively and DAPI to mark the nuclei. Yellow, double positive cells indicate developing TECs. Scale bar=20 μm.

FIG. 2D are representative micrograph immunofluorescence images of TEP grafts stained for mouse T-cell markers Cd4 (green) and Cd8 (red), respectively and DAPI to mark the nuclei. Yellow, double positive cells indicate developing T-cells.

FIG. 3A is a graph presenting principle component analysis (PC) of bulk RNA-seq samples showing clustering of day 20 TEPs, in vivo generated TEC grafts, and primary neonatal human thymus (TEPs n=2, grafts n=4, primary thymus n=2).

FIG. 3B is a dendrogram showing whole genome hierarchical clustering of bulk RNA-seq samples.

FIGS. 3C-3E present data on the significance analysis of differential gene expression displayed as volcano plots of primary neonatal thymus, TEC grafts and day 20 TEPs with third pharyngeal pouch and key thymus markers annotated. Significant P-values<0.01, minimum fold change (FC) 0.5 log. (FIG. 3C) Graft versus day 20 TEPs. (FIG. 3D) Primary neonatal thymi versus day 20 TEPs. (FIG. 3E) Primary neonatal thymus versus graft.

FIG. 3F are representative micrographs showing immunohistochemical staining for mouse Cd3 (brown) and mouse Cd45 (red) on control and TEP engrafted mouse spleens (n=2 sham, 4 engrafted).

FIG. 3G shows a bar graph of total splenocyte count in control and TEP engrafted nude mice (n=3 Sham, 7 engrafted).

FIG. 3H are representative flow plots of activation markers Cd25 and Cd69 of in vitro stimulated T-cells isolated from the spleens and lymph nodes of control and TEP engrafted nude mice (CD25: n=5 sham, 9 engrafted; CD69: n=4 sham, 9 engrafted).

FIGS. 4A-4C show t-Distributed Stochastic Neighbor Embedding (tSNE) visualization of single cell mRNA sequencing data (scRNAseq) of TEP grafts and primary human neonatal thymus samples. (Grafts n=2 (17 weeks), primary thymus n=1 prepared by two different methods). (FIG. 4A) Cluster analysis revealed distinct cell populations within samples. (B) Species-wise visualization of clusters corresponds to cluster labeling in 4A. (FIG. 4C) Sample-wise visualization the scRNAseq data shows overlap of graft derived and primary thymus derived TEPs/TECs.

FIG. 4D is a curated heatmap of common immune related genes.

FIG. 4E are violin plots showing cluster specific gene expression of key thymic markers.

FIG. 4F are violin plots showing cluster specific gene expression of T-cell markers.

FIG. 4G are violin plots showing cluster specific gene expression of key dendritic and antigen presenting cell markers.

FIG. 5A is a tSNE visualization of original cluster subpopulations with TEP and TEC clusters demarcated.

FIG. 5B is a tSNE visualization following sub-cluster analysis of TEP/TEC populations.

FIG. 5C is a sample-wise tSNE visualization of sub clustered TEC populations.

FIG. 5D is a velocity analysis showing projected developmental directionality of TEC populations.

FIG. 5E is a pseudo-time analysis of TEP subpopulations showing sample specific developmental trajectory.

FIG. 5F is a branch point heat map showing genes differentially expressed at the branching point of the pseudo-time analysis.

FIG. 5G are violin plots showing gene expression distribution of key thymic markers within the TEP/TEC populations.

FIG. 6A is a tSNE visualization of original clusters with mouse T-cells demarcated.

FIG. 6B is a tSNE analysis showing sub-clustering of mouse derived T-cells.

FIG. 6C is a pseudo-time analysis of the mouse derived T-cells.

FIG. 6D is a branch point heat map showing genes differentially expressed at the branching point of the pseudo-time analysis.

FIG. 6E shows gene specific pseudo-time trajectory of key markers of developing T-cells.

FIG. 6F are violin plots showing gene expression distribution of key transcription factors and cell surface markers of developing T-cells Panels A-B are graphs of example presence of T-cells after 6 weeks as STOCs.

FIG. 7A is a schematic of work flow for reprogramming of peripheral blood mononucleated cells (PBMCs) into patient-specific induced pluripotent stem cells (iPSCs).

FIG. 7B schematic of FOXN1 gene and strategy for insertion of HA, P2A, and Clover sequences.

FIG. 7C results from experiments outlined in FIG. 7B.

FIG. 7D shows immunofluorescence analysis of pluripotency marker proteins OCT4 (green), SOX2 (red), and NANOG (magenta).

FIG. 7E is a qPCR analysis of pluripotency marker genes OCT4, SOX2, and NANOG in iPSCs. Data is shown as the mean of technical replicates. Values were normalized and set relative to ACTB. Human embryonic stem cells served as positive control.

FIG. 7F shows karyotype analysis of established iPSC lines.

FIG. 8A is a schematic of direct differentiation approach for iPSCs to TEPs.

FIG. 8B is a table of the differentiation conditions tested and the factors used.

FIG. 8C shows gene expression analysis of third pharyngeal pouch markers, HOXA3 and EYA1, and TEP marker FOXN1, at day 16 of differentiations. Data is normalized to ACTB and shown as the mean with error bars indicating the standard error of the mean (SEM) and set relative to experimentally matched dO iPSCs (n=5, T-6 n=1). P-values, as determined by one-way ANOVA with Tukey's multiple comparisons test using ddCt values: HOXA3=<0.0001; EYA1 ****=<0.0001, **=0.0047; FOXN1 ****=<0.0001, ***=<0.0004.

FIG. 9A is a heatmap analysis of top 10 genes expressed in each of the primary clusters.

FIG. 9B is a curated heatmap of key marker genes of T-cell development.

FIGS. 9C-9E are gene specific tSNEs corresponding to gene specific violin plots in FIG. 4.

FIG. 10A is a heatmap of top 10 differentially expressed genes for each cluster.

FIG. 10B is a curated heatmap of key T-cell markers for human and mouse cells.

FIG. 10C and FIG. 10D are gene specific tSNE visualization for key thymic markers, corresponding to the gene specific violin plots in FIG. 5.

FIG. 11 gene specific tSNE visualizations for thymic genes.

FIG. 12A is a heatmap of the top up and down regulated genes in identity groups 0-9.

FIG. 12B are gene specific tSNE visualization of markers in FIG. 12A.

FIG. 12C schematic representation of enhanced T-cell receptor sequence heterogeneity in athymic mice receiving iPSC-derived graft TEP cells (Transplant) versus Control mice.

### DETAILED DESCRIPTION

Describe herein are compositions, methods, and systems for a universal direct differentiation protocol that generates thymic epithelial progenitor cells (TEPs) from various different iPSC (patient-specific, induced pluripotent stem cell) lines. The iPSCs may be derived various sources, including from human peripheral blood mononuclear cells. Upon transplantation into a recipient (for example a mammal, such as a human in need of such treatment, or an athymic nude mouse), the disclosed TEPs further differentiate into thymic epithelial cells (TECs). The grafted TECs are functional and can facilitate the education of developing mouse T-cells.

The presently disclosed methods and systems result in iPSC-derived, patient-specific TECs that are indistinguishable from TECs present in human primary neonatal thymus tissue. Single cell RNA sequencing analysis demonstrates that grafting of iPSC-derived TEPs results in the generation of all mature TEC phenotypes found in primary thymus.

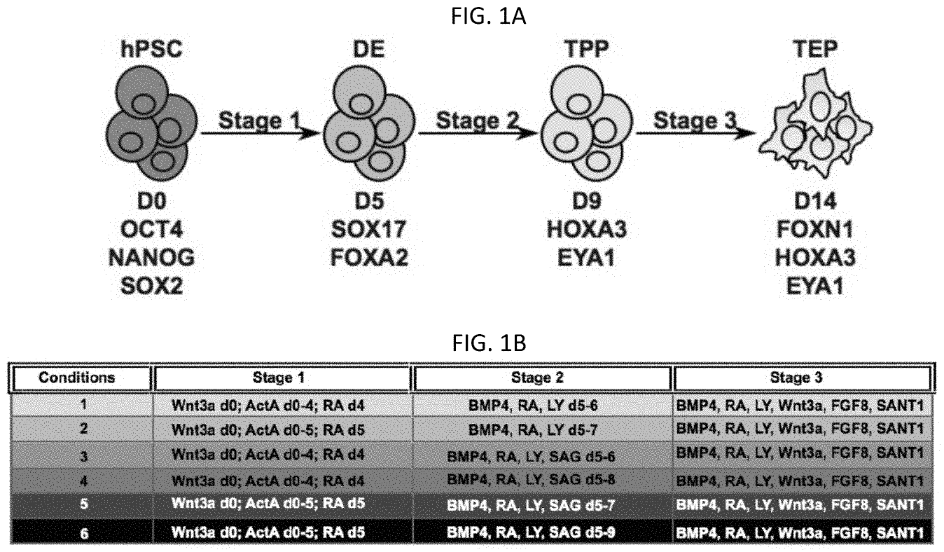

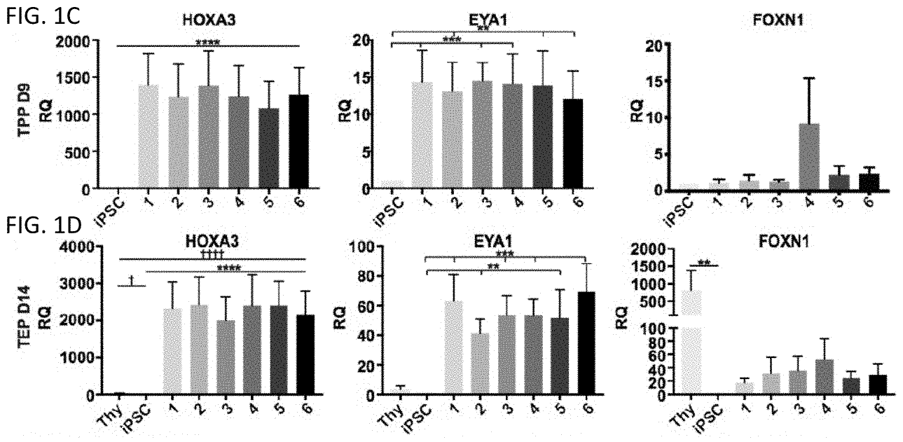

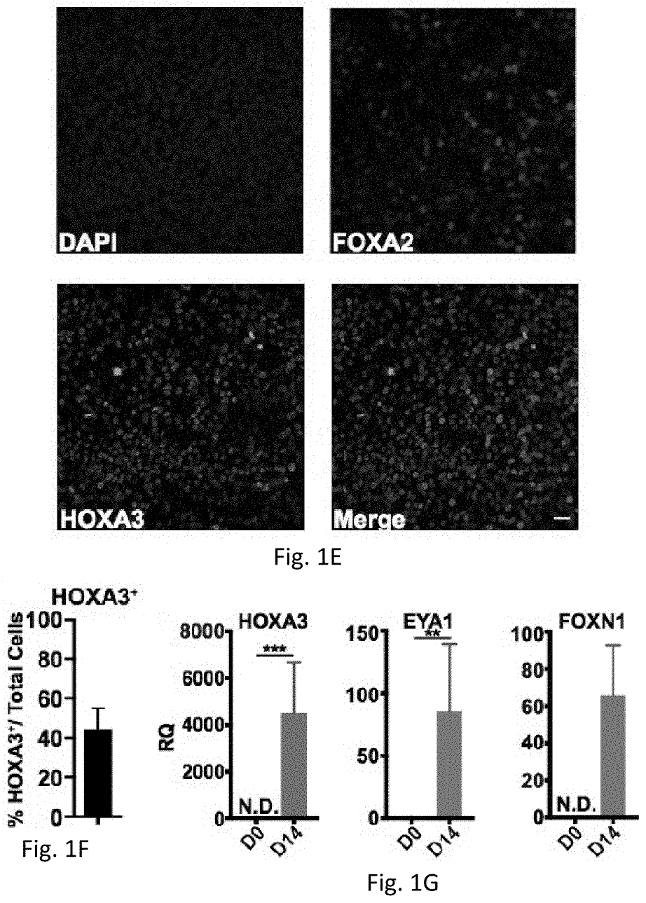

Applicants show that the present methods, systems, and compositions provide for a universal, direct differentiation protocol that generates TEPs from various iPSC lines (described schematically in FIG. 1A). The presently disclosed cells, methods, and systems can be further used to generate mature, functional thymic epithelium in a subject. In some embodiments, the disclosed methods and systems may be useful in generating a functional autogenic thymus in subjects suffering from non-existent, damaged, dysfunctional, diseased, or aged thymus.

### Thymus

The thymus is a glandular organ that is essential for the generation of a functional adaptive immune system by providing positive and negative selection of developing T-cells. The thymus is an endodermal derived tissue and originates from the third pharyngeal pouch (TPP) during embryonic development. Thymic epithelial progenitor cells (TEPs) in the TPP can be identified by the expression of the master transcription factor FOXN1 marking the thymic anlage that is surrounded by supporting mesenchymal cells. FOXN1, is necessary for the development of TEPs, and subsequently, a functional thymus. In both mice and humans, disruption of FOXN1 causes congenital athymia. Loss of Foxn1 in the adult thymus results in thymic atrophy, similar to that observed in aged individuals. While the specification of TEPs is independent of Foxn1 expression, Foxn1 is required for the differentiation of functional thymic epithelial cells (TECs) from TEPs. In addition, differentiation of TEPs into functional thymic epithelial cells (TEC) depends on interaction with developing T-cells. Functional TECs can be divided into two distinct subtypes based on their location and function; cortical and medullary TECs (cTECs and mTECs, respectively) and can be identified by the expression of cytokeratin 8 and 5, respectively. Both TEC subtypes originate from a common TEC progenitor marked by co-expression of both keratins. Developing T-cells, marked by co-expression of CD4 and 8, are first positively selected for successful interaction with the self-peptide bearing human leukocyte antigen (HLA) complex proteins on cTECs before migrating into the thymic medulla as single positive CD4 or CD8 T-cells. This process is termed positive selection and only T-cells that strongly interact with HLA/peptide receive a survival signal while the majority of developing T-cells undergo death by neglect. In the thymic medulla, mTECs are essential in the establishment of central immune tolerance through the process of negative selection by presenting self-antigens to positively selected T-cells. Autoreactive T-cells that interact with self-antigens too strongly are eliminated, while non-reactive, functional T-cells emigrate into the periphery. Absence or altered negative selection results in the development of autoimmune diseases, including type 1 diabetes (T1D). While the thymus is very active in young individuals, the organ starts to involute around the age of adolescence and naive T-cell output declines rapidly. Thymic involution is accelerated by certain clinical treatments, including chemotherapy. The ability to generate a patient-specific human thymus would provide an attractive platform the investigate human thymus biology and stimulate the development of novel model systems and treatment modalities for multiple approaches. Indeed, the majority of our current knowledge on thymus development and function is based on studies of animal models and needs to be translated and expanded in the human context for a better understanding that will allow the development of novel treatments.

Thymic epithelial progenitors (TEPs) can be generated by directed differentiation of human embryonic stem cells. The ability to effectively differentiate patient-specific TEPs from disparate iPSC lines has yet to be demonstrated in detail. Disclosed herein is the use of systematic evaluation of key signaling pathway manipulation in the development of a universal protocol for the directed differentiation of TEPs from patient-specific iPSCs. Patient-specific TEPs are further differentiated into functional TECs. These functional TECs are shown to possess the ability to educate developing T-cells upon in athymic nude mice. Single cell RNA sequencing (scRNAseq) analysis revealed that these functional, iPSC-derived TECs are indistinguishable from TECs present in primary neonatal thymus tissue, suggesting that the disclosed TECs possess a mature phenotype. The disclosed cells, methods, protocols and systems provide critical platforms for the development of innovative technologies and capabilities to investigate and model thymic function in a patient-specific manner. The disclosed cells, methods, and systems will provide for development of novel treatment modalities.

Disclosed herein are universal protocols, methods, and systems for use in generating thymic epithelial progenitor (TEPs) cells from induced pluripotent stem cell (including all cell lines tested). Disclosed herein, for example, are results from TEPs generated from iPSCs derived from various somatic cell sources and by diverse reprogramming modalities. When transplanted into athymic nude mice, the disclosed TEPs gave rise to functional thymic epithelial cells (TECs) having the ability to educate developing mouse T-cells. The presently disclosed iPSC-derived TECs are indistinguishable (by scRNAseq analysis) from TECs present in primary tissues, for example neonatal human donor thymi. These results provide further evidence that the disclosed compositions, methods, and systems are able to generate relevant thymic cells, which will impact basic and translational research by providing a framework for generation of patient-specific thymic cells.

The induced pluripotent cells may be obtained from various sources. In most embodiments, the iPSCs are patient-specific cell, that is cells were obtained from the patient and reprogrammed by methods well known in the art. As noted below, the source of the cell being reprogramed may be various tissues, organs, systems, etc. In some embodiments, the cell to be reprogramed is a hematopoietic cell, for example a hematopoietic stem cell or peripheral blood mononuclear cell (PBMC). In many embodiments, the disclosed methods and system may be useful in obtaining high yields cells expressing one or more third pharyngeal markers, for example HOXA3 (such as the HOXA3 protein), and/or the thymic marker FOXN1(for example FOXN1 mRNA). In many embodiments, the disclosed TEP cells may express one or more of the markers DLK1 and/or INHBA. In many embodiments, expression of one or more of the disclosed markers may be useful and/or novel for identification of human TEPs. In most embodiments, the disclosed protocols and systems for generating TEPs may be useful in further differentiation and generation of TECs.

The disclosed cells, methods, and systems may be useful in modeling and studying various developmental and functional thymic defects, through the use of patient- and/or group-specific TECs with known thymic phenotypes. In addition, the disclosed compositions, methods, and systems may be useful in generating functional human thymic epithelium for cell/tissue therapy for various patients, for example patients with congenital birth defects, age- or clinical intervention-related involution of the thymus, or to generate functional T-cells in a patient-specific, isogenic manner.

The disclosed differentiated thymic epithelial cells express various genes and markers typical of primary thymus cells (i.e. thymus cells from a subject with a functioning thymus, for example a neonatal thymus cell). In many embodiments the disclosed TECs upregulate one or more medullary TEC markers, for example KRT5, and one or more cortical TEC markers, for example KRT8. The disclosed TECs may also express and/or upregulate one or more of FOXN1, HLA-Class II molecules, for example HLA-DR4, and DLL4. In many embodiments, the disclosed TECs may show expression of various markers that is similar to that found in primary human thymus, for example the markers FOXN1, KRT5, KRT8, TP63, CBX4. In some embodiments, the presently disclosed iPSC-derived TECs may be indistinguishable, by single cell RNA sequencing or other methods, from primary human TECs.

Careful assessment of the effects of manipulating signaling pathways during the presently disclosed direct differentiation process for generating thymic cells in vitro shows that the disclosed methods are robust. For example, Applicants surprisingly find that the disclosed methods and systems are useful in establishing an anteriorized definitive endoderm cell population at day 5, despite subjecting cells to different manipulations. Specifically, as shown in FIG. 1D, the disclosed methods and systems resulted in cells expressing high levels of third pharyngeal markers (FIG. 1D). Quantification of the TPP marker HOXA3 protein revealed that approximately 40% of cells differentiated with the disclosed methods express this marker. These results are two-fold: first, showing reasonable induction of the target tissue, and second, highlighting possible optimization of the disclosed methods and systems to achieve higher efficiencies at this differentiation step (FIGS. 1E and 1F).

Disclosed herein are experiments where TEPs, produced from two independent iPSC lines, were transplanted into athymic nude mice. The TEPs were deposited under the kidney capsule of the mice, and later evaluated for maturation by different parameters. As is well known in the art, Nude mice are athymic, due to the absence of functional Foxnl protein, but contain HSCs that can give rise to T-cells if a functional thymus, either human or mouse, is provided. In various embodiments, the disclosed cells may be administered to the recipient in various ways. In some embodiments, the TEP cells may be implanted into various locations in the recipient. In some embodiments, the TEP cells may be implanted into the kidney capsule, intra-muscularly, for example the thigh, sub cutaneously, intra-peritoneally, the omentum, the liver (for example by perfusion, such as via the portal vein), between the liver lobes, and/or the upper anterior of the chest, beneath sternum.

The grafts, containing iPSC-derived transplanted TEPs, were analyzed for the presence of keratin 5 and 8. These studies revealed areas within the graft reminiscent of developing human thymic tissue. Specifically, the grafts possessed characteristic double positive, developing TECs, as well as more mature single positive TECs. Developing T-cells, double positive for mouse CD4 and CD8 were also found in proximity of developing thymic structures, demonstrating that the TECs are functional and capable of educating mouse T-cells. More single positive T-cells were found in the periphery of graft-bearing mice consistent with a functional thymus that increase the frequency of such cells, compared to control nude mice.

The disclosed cells may express various markers that are typical of mature, differentiated TEC cells. In some embodiments, the markers expressed may be one or more of FOXN1, HOXA3, EYA1, AIRE, CK5, CK8, CXCL12, CCL25, DLL4, and HLA-DR. In various embodiments, the marker may be differentially expressed compared to that marker in a reference cell under similar conditions (for example an in-vitro cultured TEP cell may be compared to an in-vivo grafted TEC cell) by more than about 1×, 2×, 3×, 4×, 5×, 6×, 7×, 8×, 9×, 10×, 20×, 30×, 40×, 50×, 60×, 70×, 80×, 90×, 100×, 200×, 300×, 400×, or 500×, and less than about 1000×, 500×, 400×, 300×, 200×, 100×, 90×, 80×, 70×, 60×, 50×, 40×, 30×, 20×, 10×, 9×, 8×, 7×, 6×, 5×, 4×, 3×, or 2×. In some embodiments, the reference or disclosed cell may be selected from a neonatal thymus cell, an ungrafted TEP cell, and a grafted TEC. For example, the marker may be expressed 10× more in TEP cells compared to grafted TEC cell or 10× more in grafted TEC cell compared to TEP cell.

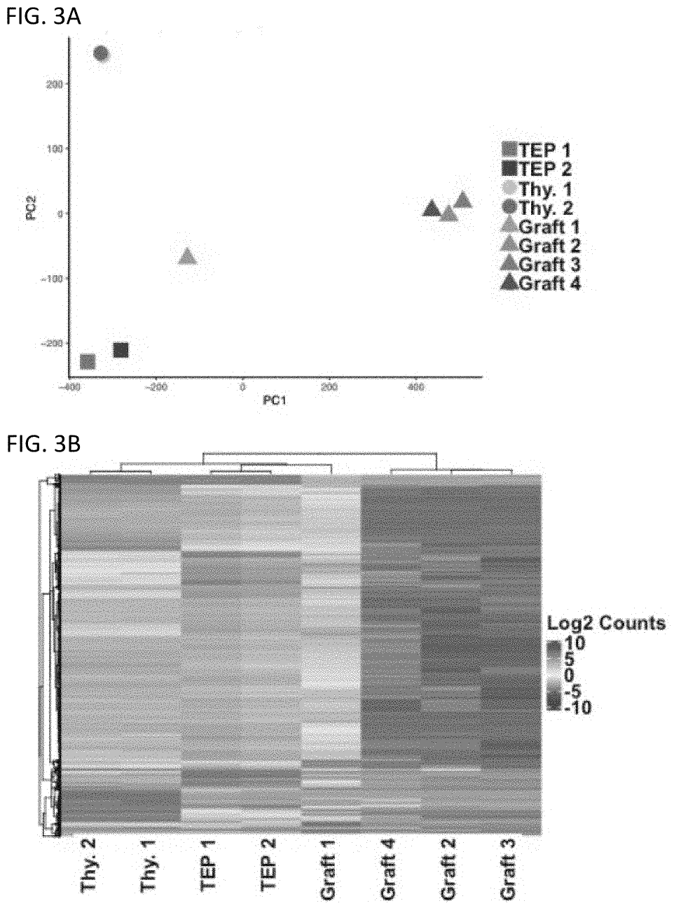

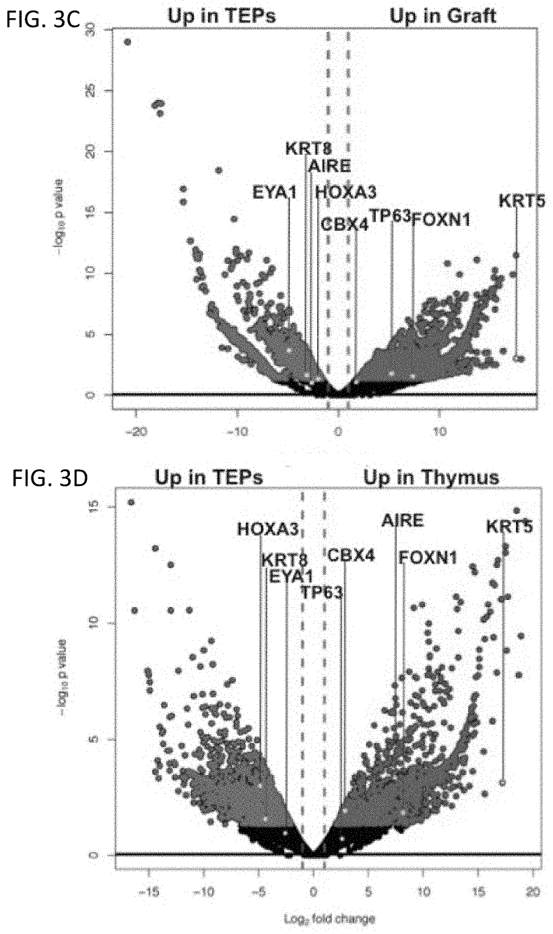

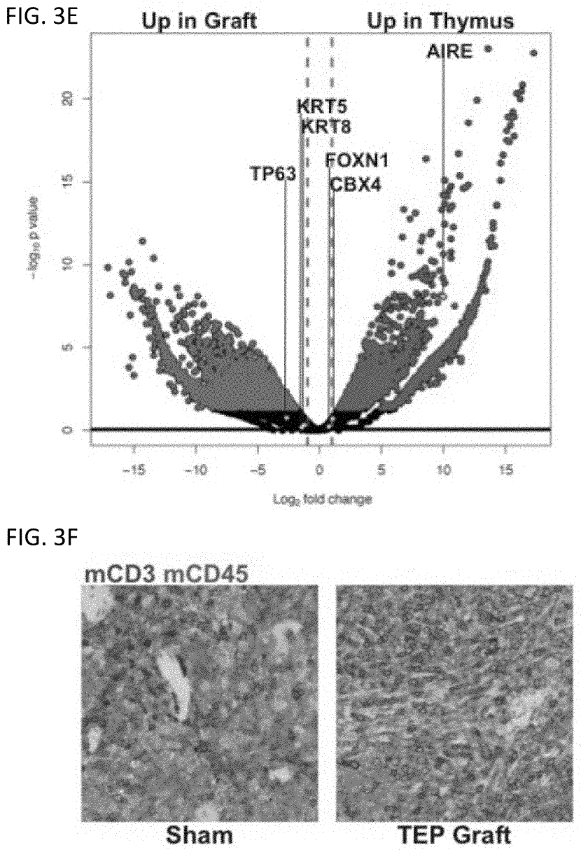

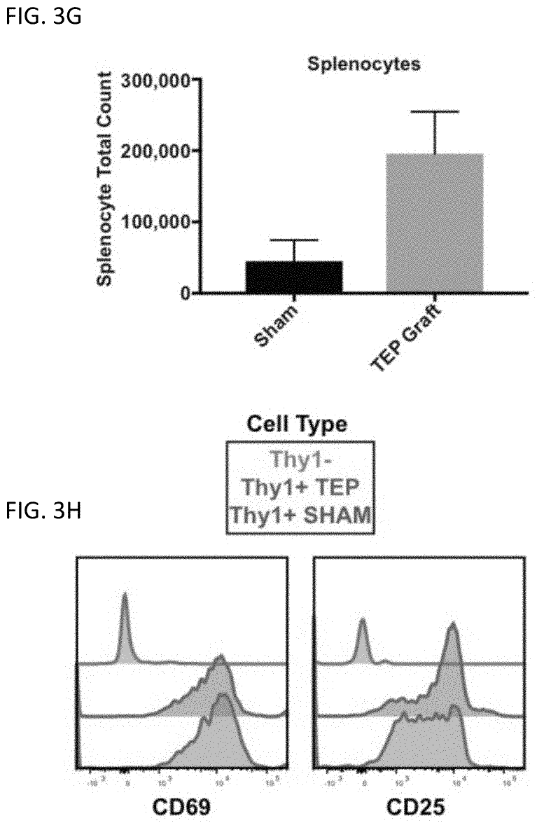

Comparative global transcriptomic analysis of in-vitro generated iPSC-derived TEPs before transplant, 4 TEC grafts from two different iPSC lines, and 2 primary human thymi further confirmed that the transplanted cells differentiated into functional TECs. For example, specific TEC markers were significantly enriched in the iPSC-derived TECs compared to TEPs, while expression levels were similar to those of primary human thymi (FIG. 3C-E). One exception is the thymic gene AIRE, critical for negative selection of developing T-cells, which was absent in the grafts. Without wishing to be limited by theory, Applicants note this absence may be due to xenograft interactions of human TECs with mouse T-cells. Notably previous studies, employing hES-derived TEPs for transplantation, also failed to detect AIRE expression. While bulk RNA sequencing for whole transcriptomic analysis allows the detection of even low-expressed genes, by increasing sequencing depth, it was unable to de-convolute heterogeneity within samples assayed.

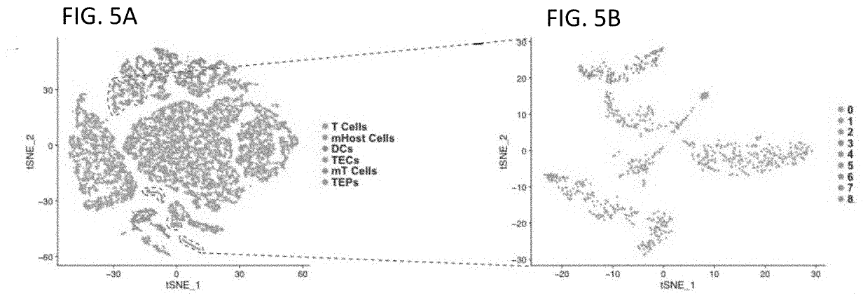

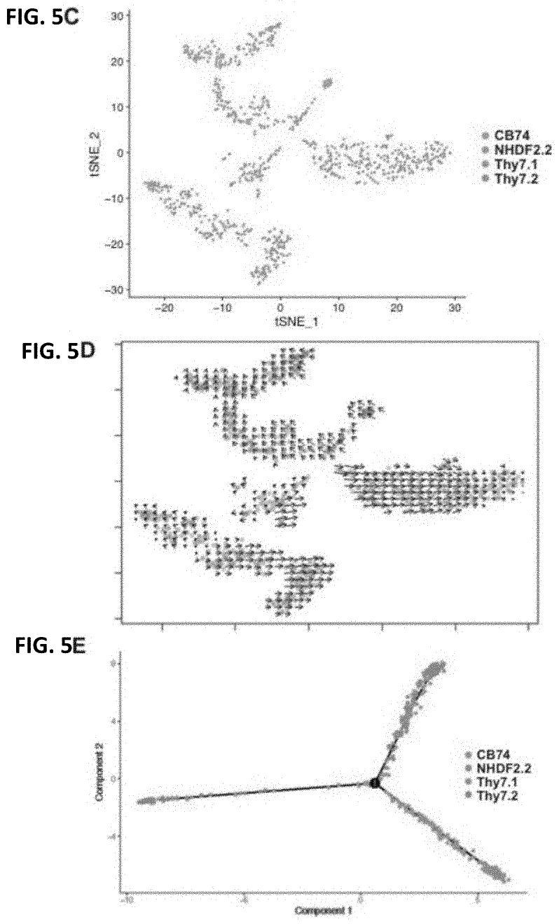

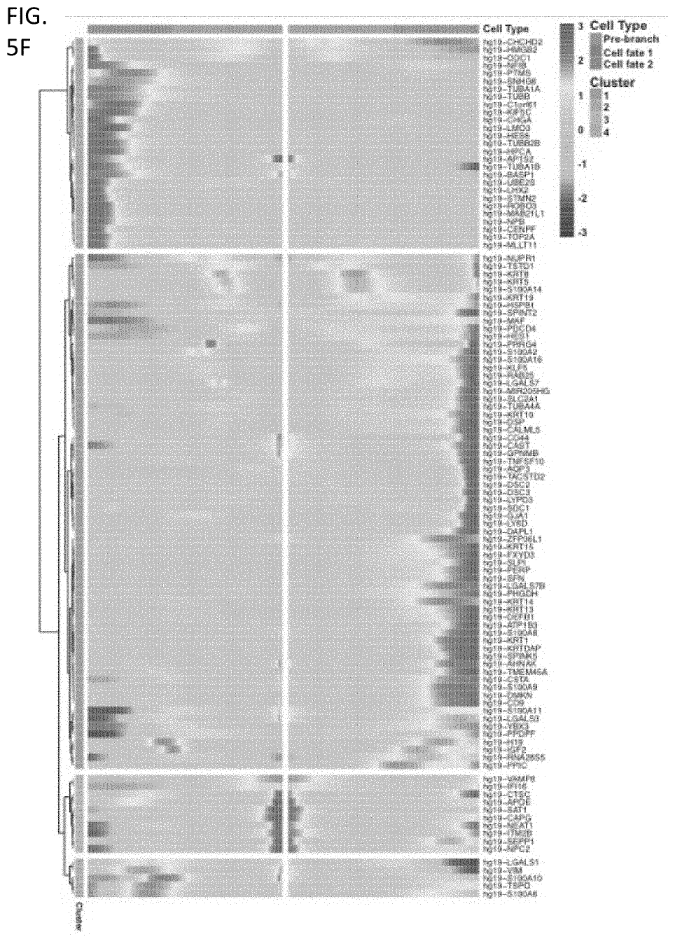

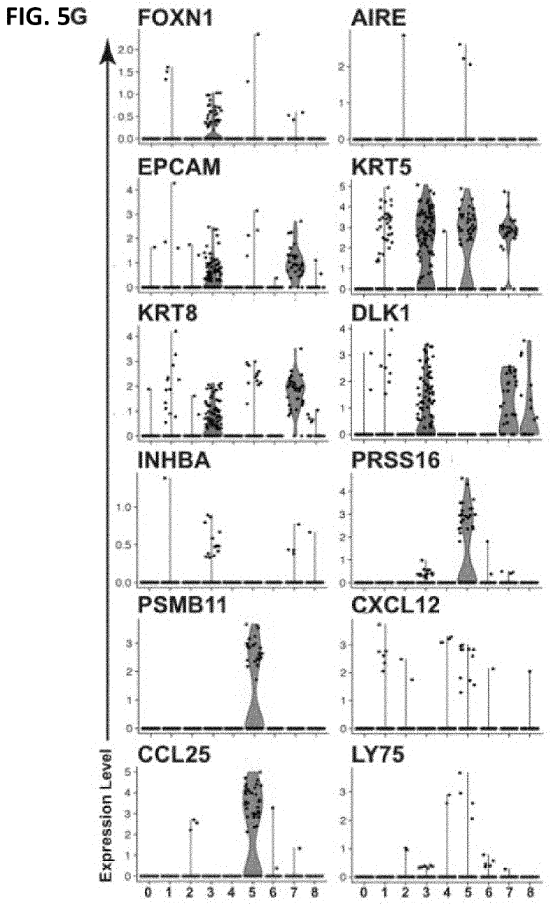

scRNAseq was employed on individual cells from grafts and the results compared to primary neonatal thymus. This allowed the identification of the expression profiles of thousands of individual cells, thus allowing classification of different cell types within samples with high confidence. Unsupervised cluster analysis was used to define specific subpopulations within the sample grafts, and marker expression profiles were used to assign specific functional cell classes to cluster subpopulations. Bioinformatic analysis was used to further differentiate between human and mouse cells with high confidence. Both mouse and human cells were found in TEC grafts from xenografts, as expected, but no mouse cells were found in primary thymus. The majority of mouse cells present transcriptomes that resemble supporting mesenchymal cells, but antigen presenting dendritic cells and, most importantly, developing T-cells were also identified. The majority of cells assayed from primary thymus were developing T-cells, characteristic TECs were identified and cluster together. Additionally, other subpopulations were identified, including dendritic and mesenchymal cells that are known to be present in thymus. These results demonstrated that a small, but appreciable, TEC subpopulation is present in iPSC-derived grafts and that these cells cluster together with primary TECs. (FIGS. 5A and C). Importantly, iPSC-derived TECs express key markers of mature TECs such as FOXN1, AIRE, EPCAM, KRT5, KRT8, DLK1, PRSS16, PSMB11, CXCL12, and CCL25 at levels similar to that seen in primary thymus tissue (FIG. 5G). Thus, the presently disclosed iPSC-derived TECs are indistinguishable from primary TECs by the bioinformatic analysis employed, demonstrating that the disclosed methods and systems are useful in generating functional TECs.

The generated data sets were useful in verifying that both iPSC-derived and primary TECs express characteristic marker genes and can be further divided into subclasses. This analysis suggests involvement of the Activin signaling pathway in TEP to TEC differentiation (FIG. 5G).

The disclosed cells, methods, and systems provide for universal protocol for differentiating patient-specific iPSCs into TEP cells that can be further differentiated, in vitro, into functional and mature TECs. Two indicators are useful in facilitating the generation of the presently disclosed TECs; the provision of 3D culture conditions that may provide an appropriate structure for thymic development and (subsequent) interactions with developing T-cells. In some embodiments, re-aggregation of thymic organotypic cultures (RTOCs), as previously described, may be useful. In this system, the growth of epithelial and mesenchymal compartments from primary human thymi may be supported by specific media compositions. After expansion, both cell types may be re-aggregated, together with CD34 positive cells isolated from various sources (for example cord blood), to create RTOCs. The RTOCs may be useful in supporting development of T-cells and thus represent an excellent system for studying aspects of T-cell education. There are, however, disadvantages to the use of RTOCs. Specifically, the allogenic origin of T-cell progenitors may hinder certain experiments, especially those requiring a selective, patient-specific environment.

A similar 3D strategy was recently described as artificial thymic organoids (ATOs). ATOs combine iPSC-derived embryonic mesoderm progenitors that can subsequently give rise to diverse repertoire of positively selected T-cells when co-cultured with a murine bone marrow derived stromal cell line that overexpress DLL4 (MS5-hDLL4). Applicants describe herein the use of the presently disclosed universal protocol for the generation of TEPs that may be helpful in various research efforts. In some cases, the disclosed protocols and systems may be useful in combining aspects of ongoing research projects to establish an isogenic culture system that may provide for both positive and negative selection of developing T-cells. In other aspects, the disclosed cells and protocols may be useful in generating diverse, patient-specific T-cells including regulator cells, as well as facilitating development of functional human thymic cell types. The disclosed cells, protocols, and systems may help to accelerate development of various cell therapies, and aid in basic and translational research efforts to understand both thymus and T-cell biology.

### Definitions

The term “allograft” refers to cells from a donor administered to a recipient patient or subject. The donor may be matched for certain criteria, but the administered cells are not derived from the recipient. In these cases, the administered cells may be extracted from the donor, treated and/or expanded before being administered to the recipient. In many embodiments, the graft cells may receive foreign biological matter, for example nucleic acids and or proteins, before being administered.

The term “autograft” refers to cells administered to a recipient that are derived from the recipient. In these cases, the administered cells may be extracted from the recipient, treated and/or expanded before being administered back to the patient. In many embodiments, the autograft cells may receive foreign biological matter, for example nucleic acids and or proteins, before being administered back to the recipient.

The term “xenograft” refers to cells administered to a recipient that are derived from a donor of another species.

The term “thymic epithelial cells” or “TEC” refers to specialized cells with high degree of anatomic, phenotypic and functional heterogeneity that are located in the thymic epithelium within the thymic stroma. The thymus, as a primary lymphoid organ, mediates T-cell development and maturation. TEC are further separated into cortical and medullary TEC (cTEC and mTEC) based on their localization within the thymic cortex or medulla respectively.

The term “thymic epithelial progenitor” cell or “TEP” cell refers to early descendants of stem cells that can differentiate to form one or more kinds of thymic epithelial cells but cannot divide and reproduce indefinitely.

The term “hematopoietic stem cells,” “HPSCs” or “HSC” refers to stem cells that form blood and immune cells (e.g., white blood cells, red blood cells, and platelets).

The term “pluripotent stem cell” or “PSC” refers to a cell that has the ability under appropriate conditions of producing progeny of several different T-cell types that are derivatives of all the three germinal layers (endoderm, mesoderm, and ectoderm). Examples of pluripotent stem cells are embryonic stem (ES) cells, embryonic germ stem (EG) cells, iPSC, and adult stem cells. PSCs may be from any organism of interest, including, primate, e.g., human, canine, feline, murine, equine, porcine, avian, camel, bovine, ovine, and so on.

The term “human embryonic stem cell” or “hESC” refers to cells that a) can self-renew, b) can differentiate to produce all types of cells in an organism, and c) is derived from a developing organism or is an established ES cell line which was derived from a developing organism. ES cell may be derived from the inner cell mass of the blastula of a developing organism. ES cell may be derived from a blastomere generated by single blastomere biopsy (SBB) involving removal of a single blastomere from the eight T-cell stage of a developing organism. In general, SBB provides a non-destructive alternative to inner cell mass isolation. ES cells can be cultured over a long period of time while maintaining the ability to differentiate into all types of cells in an organism. In culture, ES cells typically grow as flat colonies with large nucleo-cytoplasmic ratios, defined borders and prominent nuclei. In addition, ES cells express SSEA-3, SSEA-4, TRA-1-60, TRA-1-81, and Alkaline Phosphatase, but not SSEA-1.

The term “induced pluripotent stem cell” or “iPSC” refers to a cell that a) can self-renew, b) can differentiate to produce all types of cells in an organism, and c) is derived from a somatic cell. iPSC have an ES cell-like morphology, growing as flat colonies with large nucleo-cytoplasmic ratios, defined borders and prominent nuclei. In addition, iPSC express one or more key pluripotency markers known by one of ordinary skill in the art. iPSC may be generated by providing the cell with “reprogramming factors”, i.e., one or more, e.g., a cocktail, of biologically active factors that act on a cell to alter transcription, thereby reprogramming a cell to pluripotency, which are well known by those of skill in the art.

The term “cell line” refers to a population of largely or substantially identical cells that has typically been derived from a single ancestor cell or from a defined and/or substantially identical population of ancestor cells. The cell line may have been or may be capable of being maintained in culture for an extended period (e.g., months, years, for an unlimited period of time).

The term “endoderm” refers to the germ layer formed during animal embryogenesis that gives rise to the gastrointestinal tract, respiratory tract, endocrine glands and organs, certain structures of the auditory system, and certain structures of the urinary system.

The term “transforming growth factor betas”, “TGF-βs”, and “TGFBs” refers to the TGFB secreted proteins belonging to the subfamily of the transforming growth factor β (TGFβ) superfamily. TGFBs (TGFB1, TGFB2, TGFB3) are multifunctional peptides that regulate proliferation, differentiation, adhesion, and migration and in many cell types. The mature peptides may be found as homodimers or as heterodimers with other TGFB family members. TGFBs interact with transforming growth factor beta receptors (TGF-βRs, or TGFBRs) on the cell surface, which binding activates MAP kinase-, Akt-, Rho- and Rac/cdc42-directed signal transduction pathways, the reorganization of the cellular architecture and nuclear localization of SMAD proteins, and the modulation of target gene transcription. Of particular interest in the present invention are inhibitors of TGFB signaling, which can be readily be identified by one of ordinary skill in the art by any of a number of methods, for example competitive binding assays for binding to TGFB or TGFB receptors, or functional assays, e.g. measuring suppression of activity of downstream signaling proteins such as MAPK, Akt, Rho, Rac, and SMADs, e.g., AR-Smad, etc., as well known in the art.

The term “WNT” refers to the family of highly conserved secreted signaling molecules which play key roles in both embryogenesis and mature tissues. The human WNT gene family has at least 19 members (Wnt-1, Wnt-2, Wnt-2B/Wnt-13, Wnt-3, Wnt3a, Wnt-4, Wnt-5A, Wnt-5B, Wnt-6, Wnt-7A, Wnt-7B, Wnt-8A, Wnt-8B, Wnt-9A/Wnt-14, Wnt-9B/Wnt-15, Wnt-10A, Wnt-10B, Wnt-11, Wnt-16). Wnt proteins modulate cell activity by binding to Wnt receptor complexes that include a polypeptide from the Frizzled (Fz) family of proteins and a polypeptide of the low-density lipoprotein receptor (LDLR)-related protein (LRP) family of proteins. Once activated by Wnt binding, the Wnt receptor complex will activate one or more intracellular signaling cascades. These include the canonical Wnt signaling pathway; the Wnt/planar cell polarity (Wnt/PCP) pathway; and the Wnt-calcium (Wnt/Ca2+) pathway.

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. Treatment as used herein covers any treatment of a disease in a mammal and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some examples after the symptomatic stage of the disease.

The terms “condition,” “disorder,” and “disease” may be used interchangeably. In most cases these terms are directed to immune conditions, which may include any one or more conditions related to damaged, depleted, aged, dysfunctional, or missing thymic tissue, and/or T-cell disorders related there with, for example autoimmune disorders, and/or T-cell development disorders. In some cases, the disclosed immune disorder may be type-1 diabetes, and or other conditions that may benefit from induction or re-introduction of tolerance to self-antigens.

The terms “individual”, “subject”, “host”, and “patient” are used interchangeably herein and refer to any mammalian subject for whom diagnosis, treatment, or therapy is desired, particularly mammals, including humans.

The term “medium” in context of cell culture or the phrase “cell culture medium” or “cell medium” refer to a cellular growth medium suitable for culturing of various cells, including all cells described above, for example PS cells, DE cells, AFE cells, VPE cells, TEP cells. Examples of cell culture medium include Minimum Essential Medium (MEM), Eagle's Medium, Dulbecco's Modified Eagle Medium (DMEM), Dulbecco's Modified Eagle Medium/Nutrient Mixture F-12 (DMEM/F12), F10 Nutrient Mixture, Ham's F10 Nutrient Mix, Ham's F12 Nutrient Mixture, Medium 199, RPMI, RPMI 1640, reduced serum medium, basal medium (BME), DMEM/F12 (1:1), and the like, and combinations thereof. The medium or cell culture medium may be modified by adding one or more additives. Additives may include serum, such as, fetal bovine serum and/or serum replacement agents, such as, B27, N2, KSR, and combinations thereof, and differentiation factors, such as, activators of RA receptor, nodal, Act-A, Act-B, Wnt family members, activators of BMP signaling, inhibitors of TGF-β signaling, FGF, inhibitors of hedgehog signaling, and the like, and combinations thereof.

As used herein, “expression” and grammatical equivalents thereof, in the context of a marker (protein, gene, transcript, etc.) refers to production of the marker as well as level or amount of the marker. For example, expression of a marker or presence of a marker in a cell or a cell is positive for a marker, refers to expression of the marker at a level that is similar to a positive control level. The positive control level may be determined by the level of the marker expressed by a cell known to have the cell fate associated with the marker. Similarly, absence of expression of a marker or a cell is negative for a marker, refers to expression of the marker at a level that is similar to a negative control level. The negative control level may be determined by the level of the marker expressed by a cell known to not have the cell fate associated with the marker. As such, absence of a marker does not simply imply an undetectable level of expression of the marker, in certain cases, a cell may express the marker but the expression may be low compared to a positive control or may be at a level similar to that of a negative control.

As used herein, “marker” refers to any molecule that can be measured or detected. For example, a marker can include, without limitations, a nucleic acid, such as, a transcript of a gene, a polypeptide product of a gene, a glycoprotein, a carbohydrate, a glycolipid, a lipid, a lipoprotein, a carbohydrate, or a small molecule (for example, a molecule having a molecular weight of less than 10 kDa).

Defects in thymus development and function may result in the onset of immunological deficiencies and autoimmune diseases. Thus, the proper development of the thymus is critical in ensuring that individuals are born with the ability to recognize and respond to foreign antigens while also being tolerant to self-antigens. As of yet, very little research has been done to interrogate the mechanisms involved in the development of the human thymus. This has primarily been due to the lack of a sufficient model system for human thymic development and is further exasperated by the absence of reliable research tools, such as a quality antibody to human FOXN1. Traditionally, studies on thymus development and autoimmunity have been largely restricted to mouse models. This is predominantly due to the lack of availability of developing human thymi, and the lack of a faithful in vitro human thymus model. Tools and models necessary for the study of thymic development in a human and patient-specific manner are described herein.

The thymus is divided into two distinct compartments, the cortex and medulla, composed of phenotypically and morphologically distinct subsets of thymic epithelial cells, cTEC and mTEC respectively. cTECs and mTECs perform distinct functions in the development of T-cells, with cTECs functioning in the positive selection of developing T-cells for the expression of CD3, CD4 and CD8 surface receptors, and mTECs functioning in the process of negative selection of autoreactive T-cells. These subsets may be differentiated by the use of various markers, such as DLL4 and K8 for cTEC, and AIRE and K5 for mTEC. In some examples, the presence of these TEC subtype specific markers may be detected in STOCs.

Directed differentiation system has been established for the generation of TEP cells from hESC that may be capable of further maturation into functional TEC with the ability to support T-cell education after transplantation in vivo. When applied to iPSCs, however, the same protocol was not efficient in differentiating iPSC to TEP cells in vitro. Thus, the presently disclosed universal directed differentiation protocol is efficiently differentiating various iPSC lines derived from different sources.

The present disclosure describes a model system that derives TEP cells from iPSCs and efficiently differentiates all iPSC lines tested. In some embodiments, TEP cells may be derived from patient-specific human iPSCs. Also described is a FOXN1 reporter line wherein the reporter is appended to the endogenous FOXN1 gene. In some examples, the reporter may be a fluorescent reporter gene driven by endogenous FOXN1 expression. This system may allow visualization and/or immunoprecipitation of FOXN1 for various purposes. In some embodiments, FOXN1 can be tagged to aid in analysis of chromatin binding in developing TEP cells and/or isolation of FOXN1+ cells for gene expression analysis.

The disclosed differentiation protocols and systems may have applications in conferring donor-specific immune tolerance to allografts.

As used herein, the singular forms “a”, “an”, and “the” encompass embodiments having plural referents, unless the content clearly dictates otherwise. As used in this specification and the appended claims, the term “or” is generally employed in its sense including “and/or” unless the content clearly dictates otherwise.

### EXAMPLES

In order to illustrate the disclosure, the following examples are included. However, it is to be understood that these examples do not limit the disclosure and are only meant to suggest exemplary methods of practicing the disclosure.

Results—Generation of a Universal Protocol for the Directed Differentiation of Thymic Epithelial Progenitors from Patient-Specific iPSCs

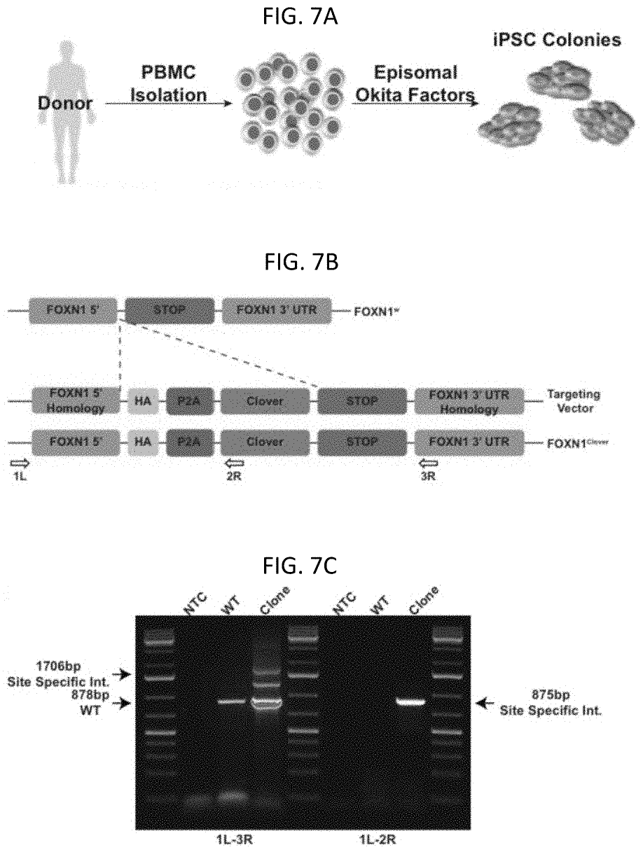

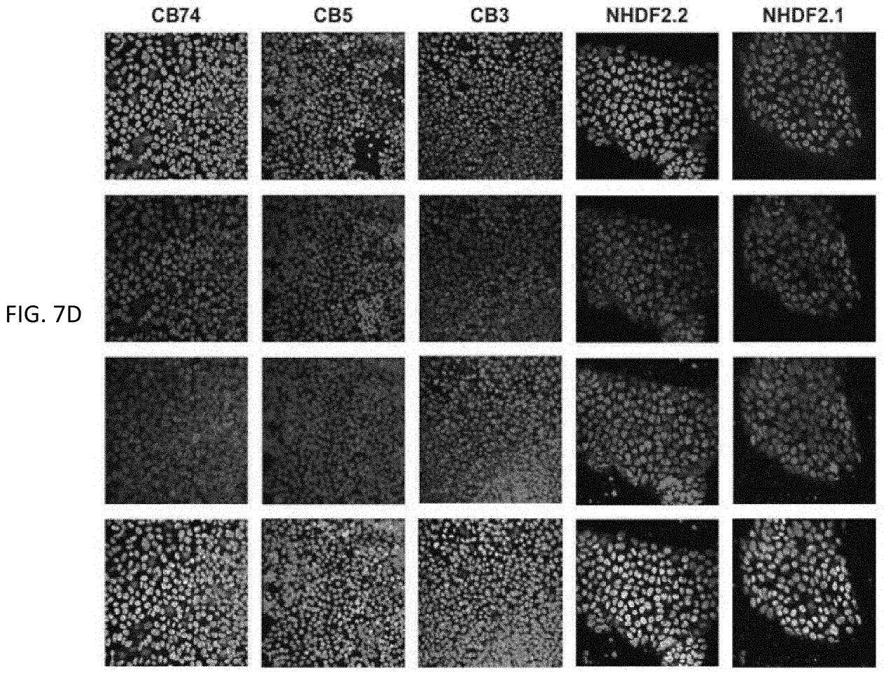

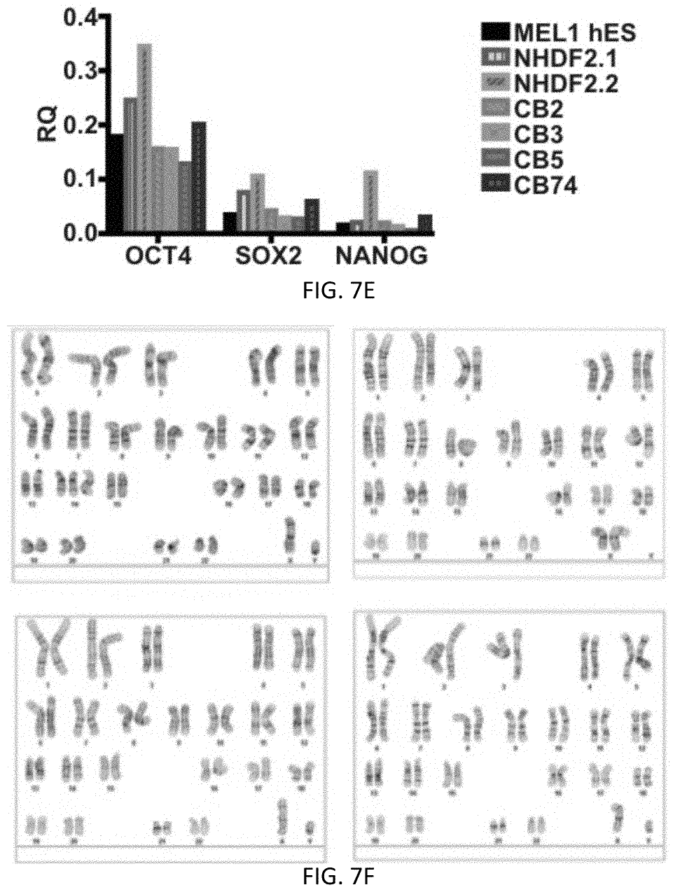

Several iPSC lines CB3, CBS, and CB74 were generated in-house from cord blood derived peripheral blood mononucleocytes (PBMCs) using non-integrative episomal vectors as described (FIG. 7A, Material and Methods at Example 1). Cell line NHDF2.1 was generated from human neonatal dermal fibroblasts utilizing RNA-based reprogramming and was described previously. Homologous recombination in conjunction with CRISPR/Cas9 technology was used to target an HA epitope tag and a nuclear localization signal tethered clover fluorescent reporter, separated by a P2A self-cleavage peptide, to the endogenous FOXN1 locus, immediately prior to the stop codon in NHDF2.1 to generate the daughter cell line NHDF2.2 (FIG. 7B). However, while the transgene was correctly targeted to the site (FIG. 7C), the fluorescence reporter was not reliable to monitor endogenous FOXN1 expression. Without wishing to be limited by theory, this may have been due to reduced protein synthesis resulting from the chosen targeting strategy. Thus, this transgene was not analyzed here. Instead, cell lines NHDF2.1 and NHDF2.2 were used interchangeably.

The disclosed compositions, methods, and systems may be useful in differentiating iPSCs into functional TEP cells, in-vitro. In many embodiments, the disclosed iPSCs may be obtained or derived from various sources including cell lines, autogenic, and allogenic sources. In many embodiments the iPSCs may be created using various methods.

Clonal iPSC lines in the present studies express pluripotency marker transcripts and proteins, OCT4, SOX2, NANOG by qPCR and immune fluorescence staining, respectively, at levels comparable to control Mel1 hESC (FIG. 7D, 7E). All, but one iPSC line assayed exhibit a normal karyotype (FIG. 7F). Specifically, cell line NHDF2.2 contains a trisomy of chromosome 14 (a common abnormality in iPSCs) in all cells analyzed by g-band karyotyping (FIG. 7F).

The generation of hESCs-derived TEPs through directed differentiation has been reported. Further to these findings, the effects of distinct changes at the definitive endoderm (DE) and/or third pharyngeal pouch (TPP) stages on FOXN1 expression at the TEP stage was analyzed. It was found that various tested conditions result in robust transcript expression of TPP markers, HOXA3 and EYA1, by day 9 of the differentiation protocol (FIGS. 1A-C). Furthermore, HOXA3 and EYA1 expression increased and remained high at day 14 of all differentiation protocols (FIG. 1D).

Of the conditions tested (and listed at FIG. 1B), Condition 4 was identified as inducing the highest levels of FOXN1 transcripts at both day 9 and 14 (FIG. 1C, D). Immunofluorescence analysis and quantification of day 9 cultures differentiated with Condition 4 showed about 45% HOXA3+ cells indicating efficient TPP generation (FIG. 1E, F).

Condition 4 differentiation, like other conditions disclosed herein, was initiated 24 hours after plating of the iPSCs. Specifically, differentiation carried out in X-VIVO10 media (Lonza 04-743Q), with the following factors were added at the indicated concentrations: Activin A, 100 ng/ml (days 0-4); Wnt3a, 50 ng/ml (d0, and d9-13); TTNPB (RAR agonist) 6 nM (d4-13); BMP4, 20 ng/ml (d5-13); LY364947, 5 μM (d5-13); FGF8b or FGF8a, 50 ng/ml (d9-13); SAG, 100 ng/ml (d5-8); SANT-1, 0.25 μM (d9-13) insulin-transferrin-selenium, 1:5,000 (d0) or 1;2,000 (d1-4). In many embodiments various supplements and/or factors may be useful in improving and/or stabilizing the generated cell phenotype. In various embodiments the supplements may be selected from Trolox, 0.1 mM; Heparin, 10 μg/ml; 2-Phospho-L-ascorbic acid trisodium salt, 50 μg/ml; hydrocortisone 0.5 μg/ml; insulin-transferrin-selenium, 1:2000; non-essential amino acids, 1×; EGF, 20 ng/ml. Supplements and factors may be available from various sources, for example Stemcell Technologies (Activin A, SAG, TTNPB), R&D Systems (BMP4 and FGF8), Novus Biologicals (Wnt3a), PeproTech (FGF8a and FGF8b), VWR (TTNPB and LY364947), Selleck Chemicals (SANT-1), Gibco (ITS and NEAA), Sigma-Aldrich (2-Phospho-L-ascorbic acid trisodium salt, heparin, hydrocortisone), Millipore-Sigma (Trolox).

The improved TEP differentiation protocol of Condition 4 was tested with 2 additional iPSC lines, for a total of three independent iPSC cell lines (NHDF2.2, CB3, CB5). qPCR analysis of day 14 cultures shows robust induction of third pharyngeal pouch (TPP) markers, HOXA3 and EYA1, and TEP marker FOXN1 (FIG. 1G). In sum, these results demonstrate establishment of a directed differentiation protocol for the efficient generation of TEPs from various human iPSCs—a universal differentiation protocol. In these experiments, universality is demonstrated through effective testing on iPSCs derived from multiple donors using different reprogramming technologies.

iPSCs may be obtained from various sources. In some embodiments the source may be skin, uterine tissue, kidney, liver, muscle, adrenal glands, blood, etc, and the cells may be neuronal cells, fibroblasts, myocytes, keratinocytes, hepatocytes, B cells, etc.

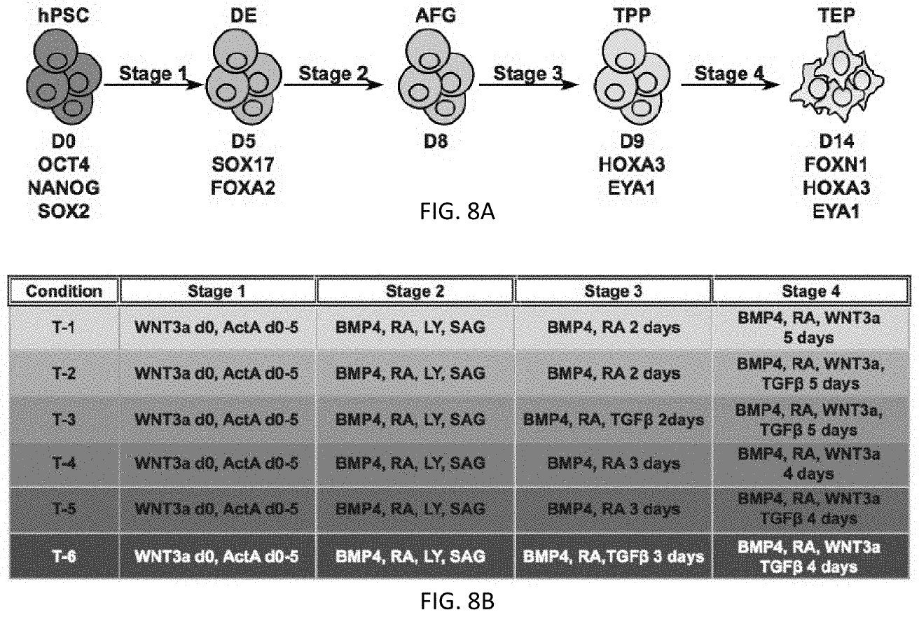

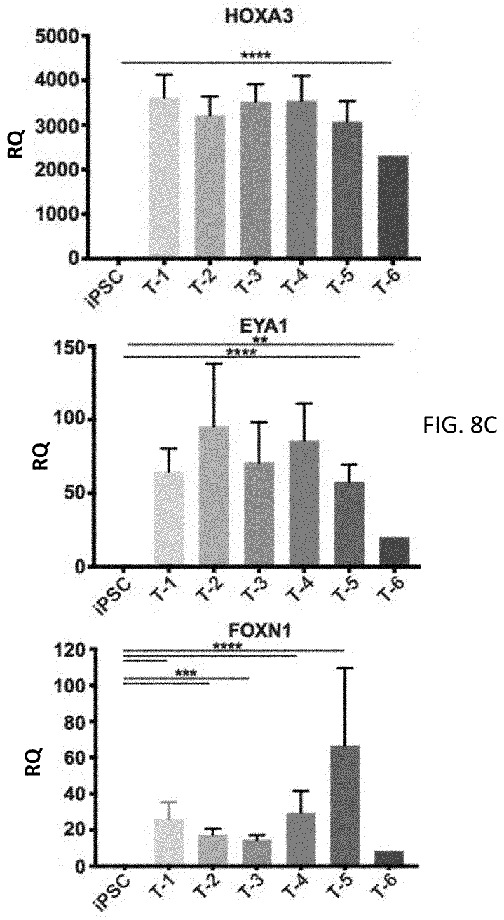

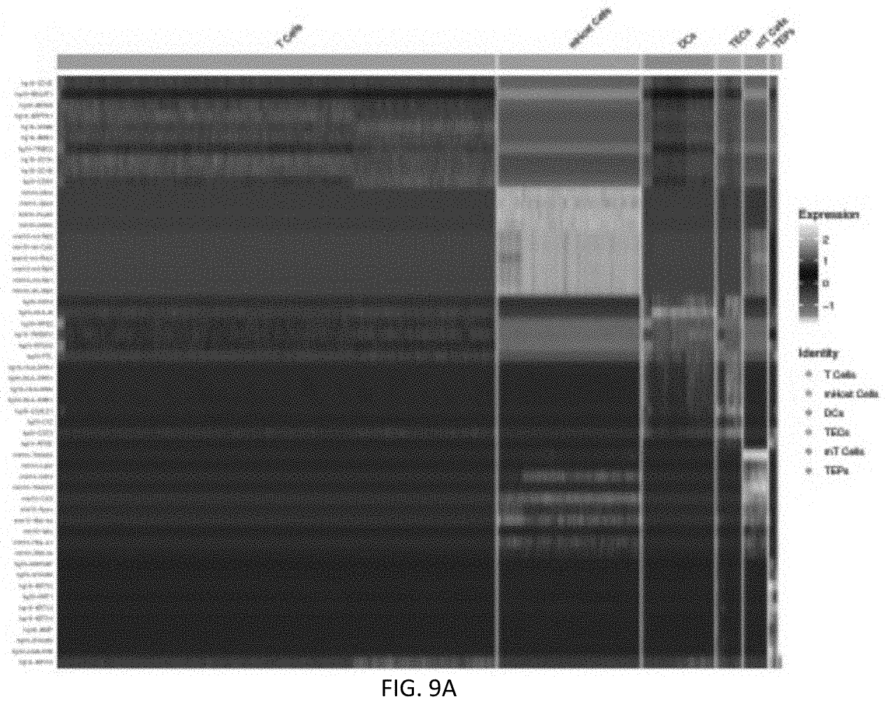

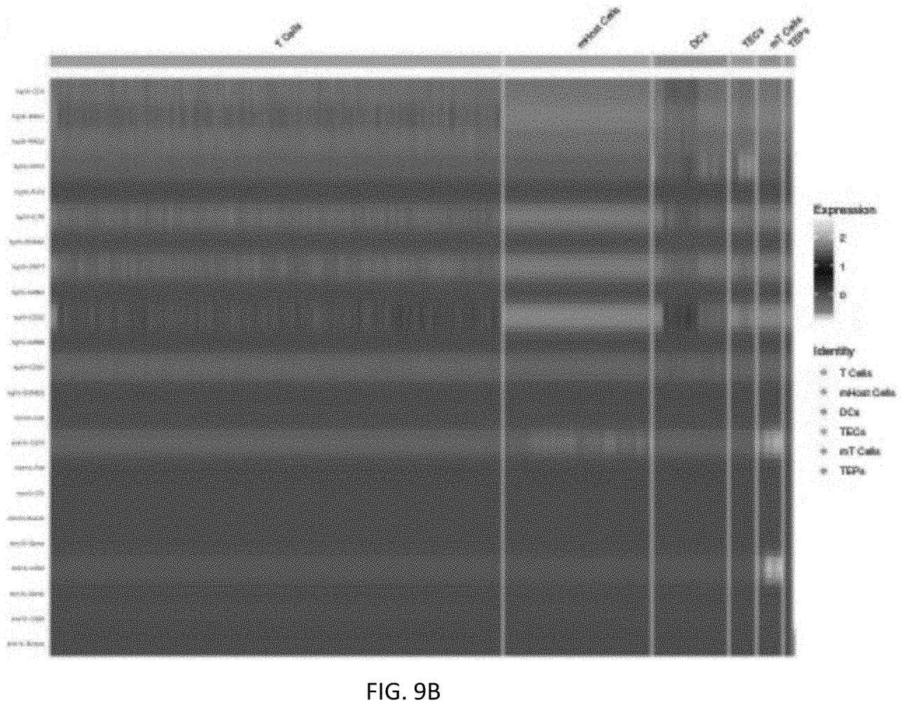

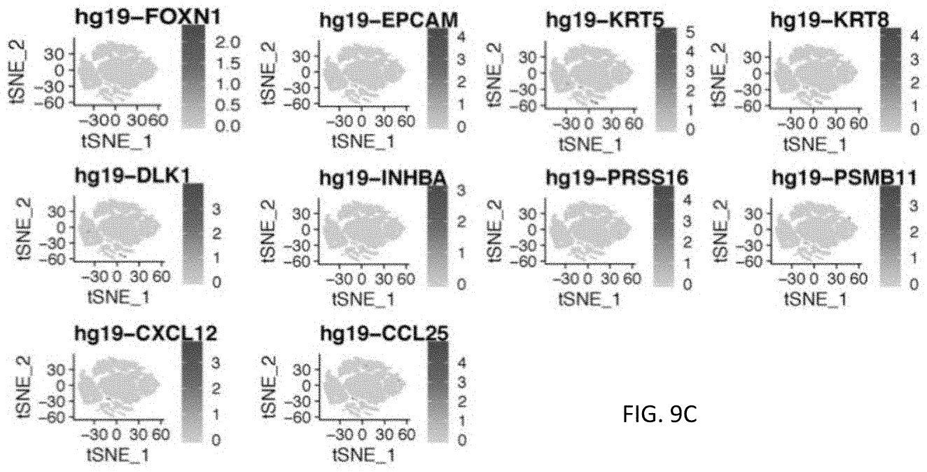

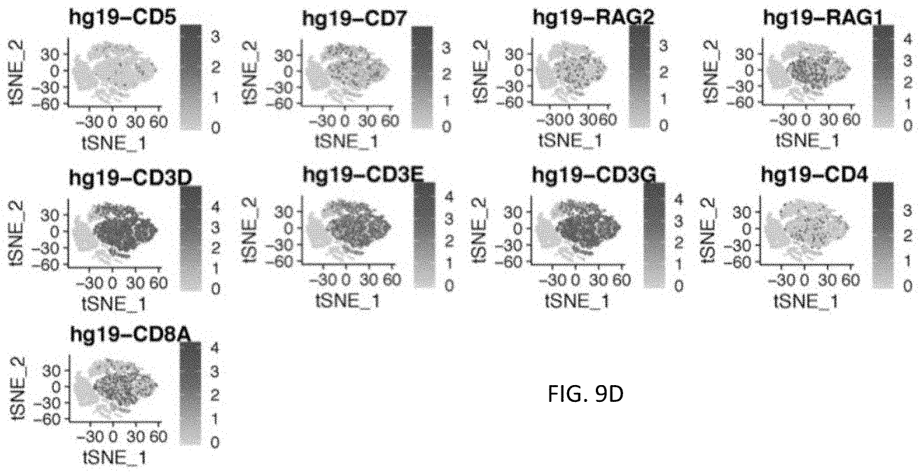

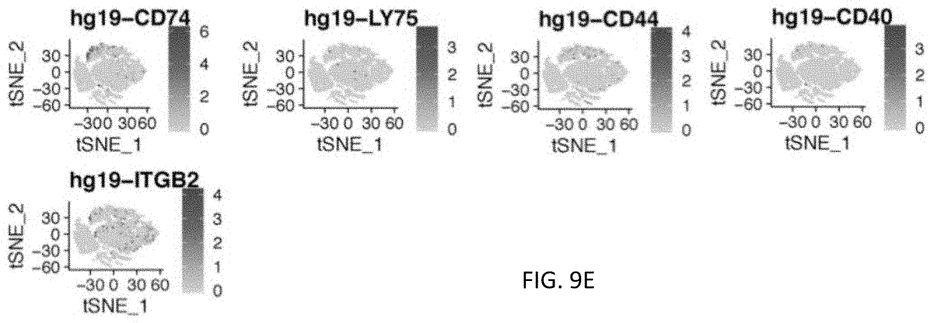

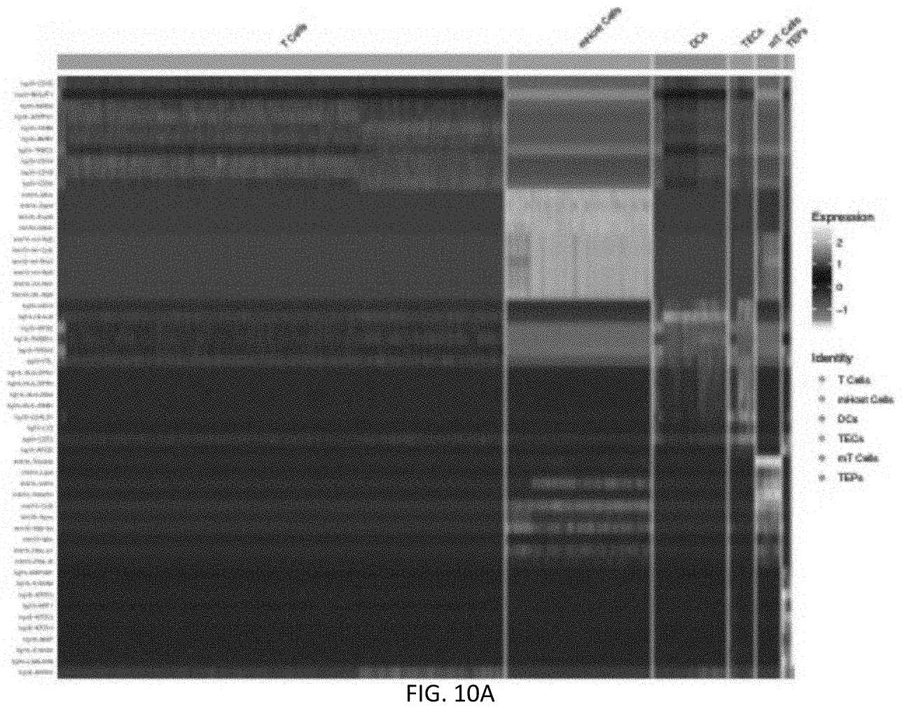

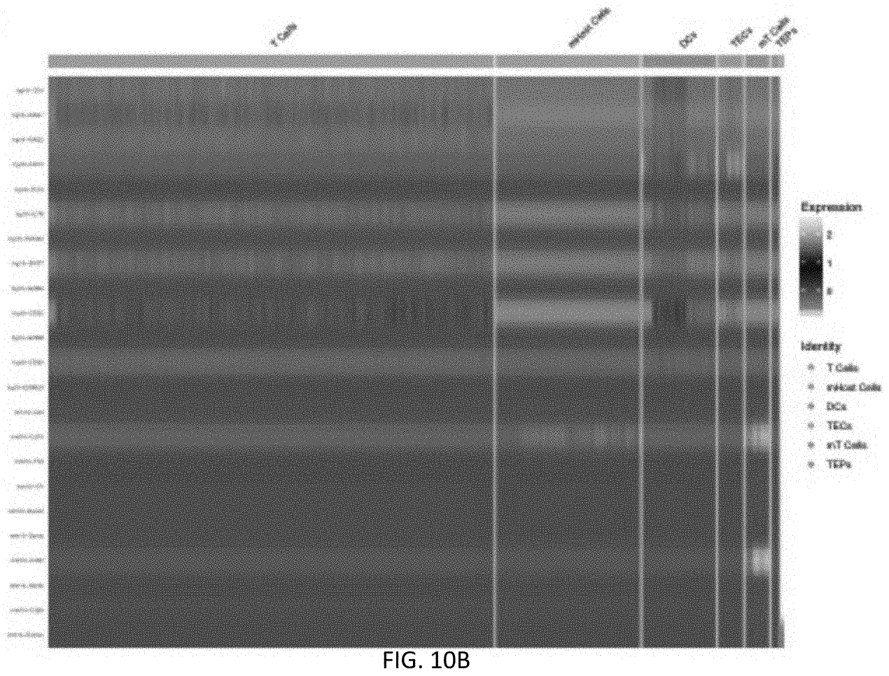

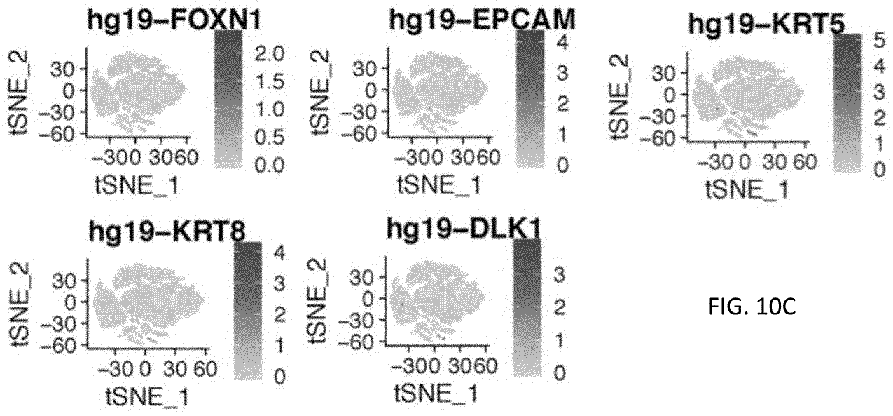

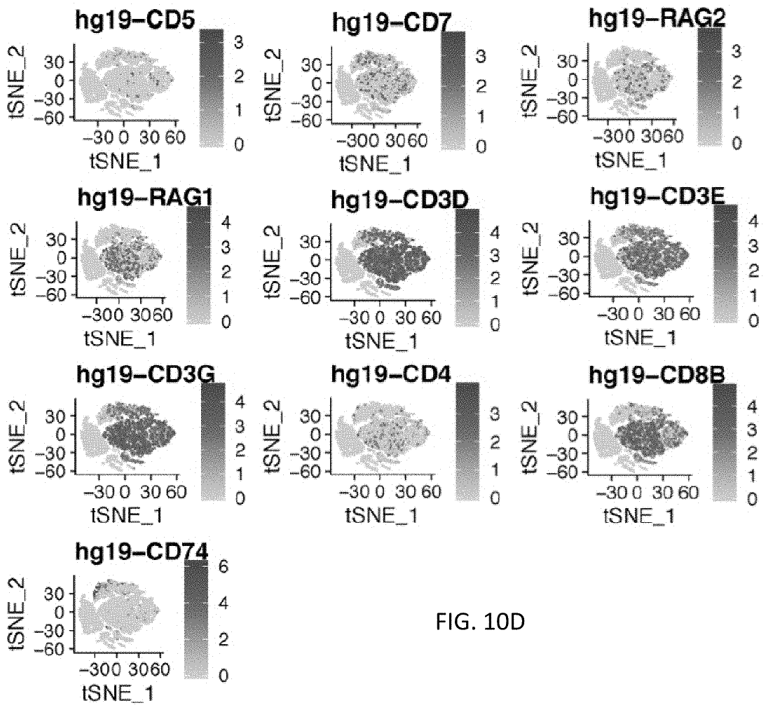

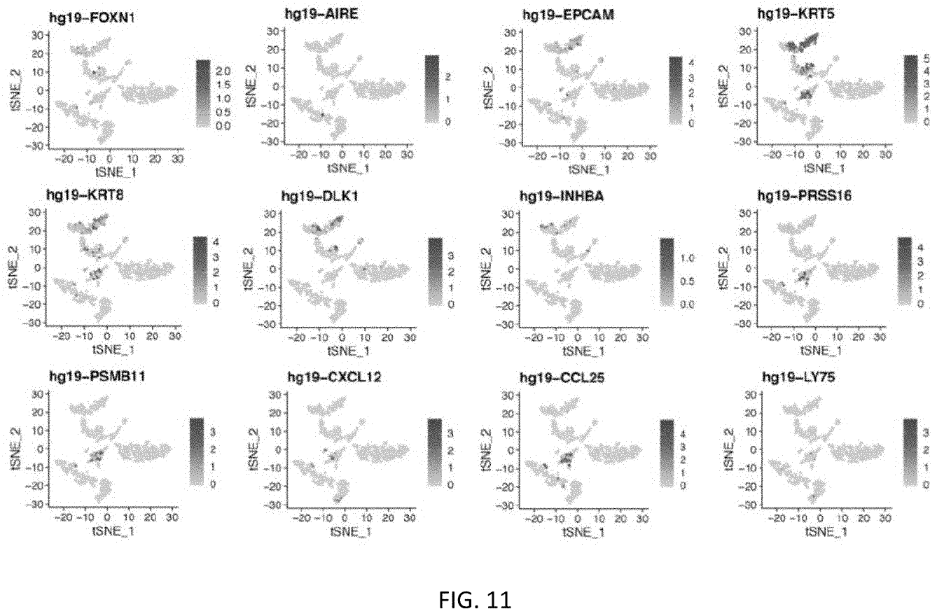

Previously, TGFβ was implicated during thymus organogenesis and function. Thus, experiments were performed to determine whether the addition of TGFβ to the TEP differentiation protocol would result in more efficient and robust generation of TEPs from iPSCs (FIG. 8A, B). The present data indicate that the addition of TGFβ during the anterior foregut endoderm (AFG) to TEP or during the TPP to TEP stages had very little effect on the differentiation efficiency of iPSCs to TEPs (FIGS. 8B and 8C). Additionally, TGFβ addition has no effect on the induction of TPP markers HOXA3 and EYA1 (FIG. 8C).

### Example 1—Materials and Methods

### Cell Culture

Undifferentiated iPSCs were maintained on Matrigel (Corning) in mTeSR1 (Stemcell Technologies), NutriStem (Biological Industries), or mTeSR plus (Stemcell Technologies), as per manufacture directions. For differentiation, iPSCs were plated on Matrigel at 3.15e5 cells/cm2. Differentiations were initiated 24 hours after plating and were carried out in X-VIVO10 (a serum free hematopoietic cell media from Lonza; 04-743Q). Factors were added at the following concentrations: Activin A, 100 ng/ml (d0-4); Wnt3a, 50 ng/ml (d0, and d9-13); TTNPB (RAR agonist) 6 nM (d4-13); BMP4, 20 ng/ml (d5-13); LY364947, 5 μM (d5-13); FGF8b or FGF8a, 50 ng/ml (d9-13); SAG, 100 ng/ml (d5-8); SANT-1, 0.25 μM (d9-13) insulin-transferrin-selenium, 1:5,000 (d0) or 1;2,000 (d1-4). Number of days reflect times used for condition 4, timing of factors may differ for other conditions as per FIG. 1B.

The addition and timing of factors in the differentiation media may vary. In some embodiments, Wnt3a is present at d0; Activin A is present at days 0, 1, 2, 3, 4, and/or 5; retinoic acid (or an analog therof) is present at day 4 and/or 5; BMP4, retinoic acid, LY364947, and/or SAG is present on one or more of days 5, 6, 7, 8, and 9; and one or more of BMP4, retinoic acid, LY364947, Wnt3a, FGF8, FGF8a, and SANT-1 is present on one or more of days 7, 8, 9, 10, 11, 12, 13, and 14. In many embodiments, the concentration of Activin A in the media may be from about 50-200 ng/ml, for example 100 ng/ml, the concentration of Wnt3a in the media may be from about 10-100 ng/ml, for example 50 ng/ml, the concentration of TTNPB (RAR agonist) in the media may be from about 2-10 nM, for example 6 nM (d4-13), the concentration of BMP4 in the media may be from about 5-50 ng/ml, for example 20 ng/ml, the concentration of LY364947 in the media may be from about 1-10 μM, for example 5μM, the concentration of FGF8b or FGF8a in the media may be from about 10-200 ng/ml, for example 50 ng/ml, the concentration of SAG in the media may be from about 50-200 ng/ml, for example 100 ng/ml, and the concentration of SANT-1 in the media may be from about 0.05-0.5 ng/ml, for example 0.25 μM.

The disclosed differentiation media may comprise one or more supplements. In various embodiments, the following supplements may be added from d9-13 for differentiations used in FIG. 1G: Trolox, 0.1 mM; Heparin, 10 μg/ml; 2-Phospho-L-ascorbic acid trisodium salt, 50 μg/ml; hydrocortisone 0.5 μg/ml; insulin-transferrin-selenium, 1:2000; non-essential amino acids, 1×; EGF, 20 ng/ml. Supplements and factors were from Stemcell Technologies (Activin A, SAG, TTNPB), R&D Systems (BMP4 and FGF8), Novus Biologicals (Wnt3a), PeproTech (FGF8a and FGF8b), VWR (TTNPB and LY364947), Selleck Chemicals (SANT-1), Gibco (ITS and NEAA), Sigma-Aldrich (2-Phospho-L-ascorbic acid trisodium salt, heparin, hydrocortisone), Millipore-Sigma (Trolox).

Collection and Reprogramming of PBMCS to iPSC and Collection of Neonatal Thymus Tissue

De-identified cord blood was obtained from the University of Colorado cord blood bank (http://www.clinimmune.com/cordbloodbank/). The use of human subjects was approved by the Colorado Multiple Institutional Review Board (COMIRB #14-0842; principal investigator AJ). Human peripheral blood mononuclear cells (PBMCs) were isolated and expanded for 6 days in StemSpan SFEM II media with Erythroid Expansion Supplement (StemCell Technologies). Erythroid progenitor cells were transduced with Okita factors using P3 Primary Cell 4D Nucleofector X Kit L (Lonza) as described previously. Transduced cells were plated on a Matrigel coated six well plate and cultured in ReproTeSR Media (StemCell Technologies) for 14 days with media changes every other day. Thereafter, cultures were fed with mTESR1 (StemCell Technologies) with daily media changes. Individual iPSC colonies were picked between days 12-18, following expansion and phenotypical analysis as outlined above. Deidentified human neonatal thymus tissue was obtained after review and approval by the Colorado Multiple Institutional Review Board as not human subject research (COMIRB #18-0347; principal investigator HAR).

### Quantitative Real-Time PCR

RNA was extracted from hPSC and TEP cultures and dissected grafts using a RNeasy micro kit (QIAGEN) per manufacturer's instructions. Reverse transcription of RNA was performed using iScript cDNA synthesis kit (Bio-Rad, 1708891BUN), as per manufacturer's instructions. Real-time quantitative PCR was performed on a CFX96 Touch Real-Time PCR Detection System (Bio-Rad) using human specific Taqman probes (Bio-Rad or ThermoFisher) or human specific primers as listed below. Samples were normalized to endogenous control gene ACTB and plotted relative to undifferentiated iPSCs.

| Probe | | SEQ |
|---|---|---|
| Target | Supplier: Assay ID | ID NO |
| ACTB | ThermoFisher: Hs01060665_g1 | |
| ACTB | ThermoFisher: Hs99999903_m1 | |
| AIRE | Bio-Rad: qHsaCIP0029272 | |
| CCL25 | ThermoFisher: Hs00608373_m1 | |
| CCXL12 | ThermoFisher: Hs00171022_m1 | |
| DLL4 | Bio-Rad: qHsaCEP0051500 | |
| EYA1 | ThermoFisher: Hs00166804_m1 | |
| FOXN1 | ThermoFisher: Hs00919266_m1 | |
| HLA-DRA | Bio-Rad: qHsaCEP0040019 | |
| HOXA3 | ThermoFisher: Hs00601076_m1 | |
| KRT5 | Bio-Rad: qHsaCEP0055058 | |
| KRT8 | Bio-Rad: qHsaCEP0041467 | |
| NANOG | Bio-Rad: qHsaCEP0050656 | |
| OCT4 | Bio-Rad: qHsaCEP0041056 | |
| SOX2 | Bio-Rad: qHsaCEP0039595 | |
| | | SEQ |
| Target | Primer Sequence | ID NO |
| ACTB F | CATGTACGTTGCTATCCAGGC | |
| ACTB R | CTCCTTAATGTCACGCACGAT | |
| NANOG F | CCCCAGCCTTTACTCTTCCTA | |
| NANOG R | CCAGGTTGAATTGTTCCAGGTC | |
| OCT4 F | CCGAAAGAGAAAGCGAACCAG | |
| OCT4 R | ATGTGGCTGATCTGCTGCAGT | |
| SOX2 F | CCATGACCAGCTCGCAGAC | |
| SOX2 R | GGACTTGACCACCGAACCC | |

### Immunofluorescence

iPSC cultures were fixed at room temperature (RT) for 15 minutes in PBS+4% paraformaldehyde (PFA), washed three times with PBS and blocked/permeabilized for 30 minutes at RT in CAS-block (Invitrogen)+0.2% Triton X-100. Primary antibodies (listed below) were diluted in CAS-block+0.2% Triton X-100 and samples were stained for 1 hour at RT. Slides were washed with PBS+0.1% Tween three times for 5 minutes and incubated with secondary antibodies (Alexa Fluor tagged secondary antibodies (Invitrogen)) diluted 1:1000 in PBS+0.1% Tween and stained for 40 minutes at RT. Slides were washed three times in PBS+0.1% Tween and once in PBS then mounted in ProLong Gold antifade reagent with DAPI (Invitrogen). For tissue sections, 4-10 μm sections were cut from paraffin-embedded tissue blocks using a microtome and placed on microscope slides. Deparaffination and antigen retrieval was performed by washing the slides 3× in Xylene for 5 minutes, 2× in 100% ETOH for 2 minutes, 2× in 95% ETOH for 2 minutes, 2× in 70% ETOH for 2 minutes, 1× in 40% ETOH for 2 minutes, 1× in H2O (tap) for 5 minutes. Antigen retrieval was performed in Tris-EDTA Buffer (10 mM Tris Base (Fisher BioReagents), 1 mM EDTA (KD Medical), 0.05% Tween 20, pH 9.0). Tris-EDTA buffer was brought to boiling and placed in rice cooker with boiling water. Slides were added to the hot Tris-EDTA buffer for 20 minutes and washed in cold tap water for 10 minutes after. Slides were then blocked and stained as described above. Z-stack images were taken with a Zeiss LSM 800 microscope. For quantification analysis, 1 field of view from each well was imaged randomly and the percentage of total HOXA3 positive cells over total DAPI positive cells was quantified by hand using ImageJ software. Antibodies are as follows, KRTS Abcam (ab52635) 1:100, KRT8 Santa Cruz (sc-8020) 1:100, HOXA3 Santa Cruz (sc-374237) 1:100, FOXA2 Millipore (07-633) 1:300, OCT4 Santa Cruz (sc-5279) 1:100, SOX2 Abcam (ab97959) 1:500, NANOG Abcam (ab77095) 1:300. Graft staining for mouse CD's was performed by the Human Immunology and Immunotherapy Initiative (HI3) at the University of Colorado Anschutz Medical Campus.

### Flow Cytometry

Collected mouse tissues were dissociated in DMEM containing 1 mg/mL collagenase IV (Worthington, Lakewood, N.J.; Cat #LS004189) at 37° C. for 1.5 h. Cells were filtered (40 μm) and red blood cells lysed in ACK buffer (Life Technologies, Carlsbad, Calif.; Cat #A1049201) before re-suspension in PBS containing 2% FBS for analysis. White blood cells were separated from whole blood by ACK lysis and centrifugation. Cells were washed and stained with the mouse antibodies CD3, CD45 at a 1:10 concentration (Biolegend, San Diego, Calif.; Cat #100235, 103105). Cytometric analysis was conducted on a CyAn ADP (Beckman Coulter, Fort Collins, Colo.) and analyzed using Summit V5.1 (Beckman Coulter) software.

### Immunohistochemistry

Slides were de-paraffinized and re-hydrated in graded concentrations of alcohol before antigen retrieval in citrate buffer, pH 6 (Dako, Carpinteria, Calif.; Cat #S1699) at 50° C. for 10 min and rinsed in wash buffer (Dako; Cat #K8007). All staining was done in a Dako Autostainer. Slides were incubated in dual endogenous enzyme block (Dako; Cat #52003) for 10 min, protein-free blocking solution (Dako; Cat #X0909) for 20 min, then in primary antibody for 60 min. Primary antibodies and dilutions: mouse CD45 (Becton Dickinson Biosciences, San Jose, Calif.; Cat #550539), 1:400; mouse CD3 (R&D Systems, Minneapolis, Minn.; Cat #MAB4841), 1:50. Staining was developed as follows: EnVision +Dual Link System HRP (Dako; Cat #K4061) for 30 min and substrate-chromogen (DAB+) Solution (Dako; Cat #K3468) for 5 min. Slides were counterstained with hematoxylin (Dako; Ct #53301) for 10 min. Quantification of cells was performed by counting staining in at least three non-overlapping fields on a Zeiss Axio Imager A2 microscope.

### T-Cell Activation Assay

Splenocytes/Lymph node cells from Sham and TEP grafted mice were prepared by lysing the red blood cells then purifying them through negative selection for CD4 and CD8 (Pan T-cell isolation kit II mouse, MACS #130-095-130). These cells were then plated and stimulated with plate-bound anti-CD3 (145-2C11, 10 ug/mL) and soluble anti-CD28 (35.11, 1 ug/mL) for 24 hours. After 24 hours cells were harvested and analyzed for their activation status by flow cytometry with antibodies against the following cell surface markers (Thy1.2 APC (BD 553007), CD25 PE (BD 553075), CD69 PE (BD 553237), CD4 FITC (BD 553729), CD8a PacBlue (Biolegend 100725)). Lymphocytes were gated on with Thy1.2 and then subgated by CD4 and CD8 to analyze cell surface expression levels of the activation markers, CD69 and CD25. Expression levels were compared to the Thy1-population as a control between the Sham and TEP grafted cells.

### Cell Preparation for Single Cell RNA Sequencing

Graft was carefully dissected from resected kidney and placed in 0.25% trypsin at 4 degrees Celsius(C.) for 2-2.5 hours. Graft and trypsin mixture was then placed at 37 C for 5 minutes, vortexed and passed through 35 μm filter, counted using a hemocytometer and diluted to a concentration of 100-2,000 cells/μl. For primary thymus samples, a small ˜1 cm3 section of tissue was dissected, minced with a razor blade, and incubated in 0.25% trypsin for 1.5 hours at 4 C. A second ˜1 cm3 section of tissue was dissected and mashed against a 35 μm filter. The tissue section was washed 5 times with PBS with mechanical agitation, in an attempt to deplete the sample of thymocytes and enrich for the epithelial cell compartment. Thymocyte depleted section was then minced with a razor blade and placed in 0.25% trypsin for 1.5 hours at 4 C for 1.5 hours. Tissue and trypsin mixture was then placed at 37 degrees C. for 5 minutes, vortexed and passed through 35 μm filter, counted using a hemocytometer and diluted to a concentration of 100-2,000 cells/μl. Cell suspensions were then taken to the UC Anschutz Medical Campus Genomics and Microarray Core for single cell sequencing on a 10× Genomics Chromium Box.

### RNA Preparation for Bulk Sequencing

For d20 TEPs, one well of a 24-well plate was collected and resuspended in 350 μl Qiagen RLT lysis buffer. For grafts, TEP grafts were dissected from resected kidney, and a small portion was placed in 350 μl Qiagen RLT lysis buffer. Graft was then homogenized using an electronic pellet pestle (Kimble). For primary thymus, a small ˜1-2 mm3 section of tissue was placed in 500 μl of RLT lysis buffer. RNA was isolated using QIAGEN RNeasy mini kit, as per manufacturer's instructions.

FOXN1 Targeting Plasmid Generation and iPSC Targeting

sgRNA sequences were designed using benchling.com: 5′-gCACAGCTCATGCCAGGGCCA-3′ (SEQ ID NO:) and 5′-GCTGGGCACAGCTCATGCCA-3′ (SEQ ID NO:). The sgRNA sequences were cloned into the PX459 plasmid (Addgene, plasmid #62988) to generate the CRISPR targeting construct. The donor plasmid (reporter cassette) carrying FOXN1 homology arm (HA), 5′HA-3×HA-P2A-mClover-3′HA, was synthesized in PUC57 (701032-1). The targeting construct was further confirmed by a restriction digestion and DNA sanger sequencing. N2#1 iPSCs were dissociated into single cells using TrypLE for 7 min at 37 C. Single cells were electroporated using the Bio-Rad Gene Pulser Xcell Electroporation system. 8 million cells were mixed in mTSER1 media with 10 uM ROCKi to which 20 ug of both CRISPR targeting constructs and 40 ug of the donor plasmid was added. Cells were plated in Matrigel-coated 10 cm plates with mTSER1 and 10 uM ROCKi. 24 hours later, cells were with puromycin (0.5 ug/ml) for two days. Two weeks later, clonal colonies were picked, expanded and prepared for gDNA. gDNA was isolated using Lysis buffer (100 mM TrisHCl, 5 mM EDTA, 2% SDS and Proteinase K) followed by precipitation using isopropanol. All colonies were genotyped by PCR using primers (see table below) to detect the integration of the reporter cassette. One primer pair, 1L and 3R, amplifies a region outside of the 5′ HR and the 3′ HR of the reporter cassette (expected bands: WT: 878 bp, Site specific integration: 1706 bp). The other primer set, 1L and 2R, amplifies a region outside of the 5′ HR and within the clover sequence of the reporter cassette (expected bands: WT: no band, site specific integration: 875 bp). One colony was found to contain successful site specific integration of the transgene by PCR of gDNA and Sanger sequencing of the PCR products, termed NHDF2.2.

| Name of | | SEQ ID |
|---|---|---|
| primer | Sequence (5′-3′) | NO: |
| 1L | AATCTACCTTCCTTGGGAGACTGG | |
| 2R | TAAACGGCCACAAGTTCAGC | |
| 3R | CCTCTCACACATTTCTGCCA | |

### Statistics

Data was analyzed using GraphPad Prism software. A one-way ANOVA was performed on the delta delta Ct values. Error bars in bar graphs represent the standard error of the mean.

### Single Cell Sequencing

Libraries was prepared and run on Illumina and 250M reads were captured for all the 4 samples. The reads were aligned using the 10× genomics, Cell Ranger pipeline to generate feature-barcode matrices. Both human and mouse references (GRCH38-and-mm10) were used to align the individual xenograft sample reads. The number of cells captured in the thymus samples was 8,515 followed by 5,500 cells in thymus-depleted, 4,042 in EEE1 and 1602 cells in HM74. The mean reads per cell ranged from 151,767 to 50,644 across the samples. The raw feature-barcode matrices of each sample were combined in R and analyzed using the Seurat (3.1.0) pipeline.

Pre-processing: The cells were filtered on the basis of number of unique genes in each cell and the percent of mitochondria present. Cells with less than 250 genes and more than 5000 genes were discarded. Cells having more than 5% human and mouse mitochondrial content were not analyzed further as higher mitochondrial content correlates with low-quality or dying cells. The data was normalized using the Log Normalize method to a scale factor of 10,000. For feature selection, variation-stabilizing transformation was applied as is detailed by Stuart et al, to return 5000 features per dataset. Next, linear transformation (scaling) was performed prior to linear dimensionality reduction.

The cells were clustered based on their PCA score, first a K-nearest neighbor (KNN) graph is constructed based on the euclidean distance in PCA space and then the cells are clustered by applying the Louvain algorithm. Non-linear dimensionality reduction was done to generate the tSNE plot. The cells were identified, and the differentially expressed genes were found using the Wilcoxon rank sum test. To find the markers for every cluster compared to the rest of the cells, Seurat function FindAllMarkers was used, the min.pct was set at 0.1 and logfc.threahold was set at 0.25, implying that the features had to be expressed by a minimum of 10% of the cells and have a log-fold change greater than 0.25.

### Pseudotime Analysis

The pseudotime analysis was done using the Monocle pipeline. The phenotype data and feature data were extracted from the Seurat object and Monocle CelldataSet class was created. First low-quality cells were filtered out to remove dead or empty wells in plate as well as doublets and triplets. This was done by setting minimum expression to 0.1 and num_cells_expressed>=10. The cells were clustered without marker genes and the differentially expressed genes were found out. Monocle uses an algorithm to learn the changes in gene expression as cells go through the biological changes and places them along a trajectory. The dimensionality was reduced, and the cells were plotted along the trajectory based on the clusters as well as the original samples. The pseudotime dependent genes were found out separately for the mT-cells and the TEPs plus TECs and plotted on a heatmap.

### Velocity Plot

The velocity plot was constructed using the Velocyto pipeline. The pipeline was run on the Cell Ranger output using the combined reference genomes to generate a loom file that has the splicing information. The embeddings were taken from the Seurat object loaded on R and the distance between the cells were estimated. The gene relative velocity of the spliced and un-spliced objects was estimated, and the velocity was shown on the Seurat tSNE embeddings.

### Bulk RNA Sequencing

RNA-seq reads were generated from the Illumina sequencing platform. Sequencing quality and adapters were checked using FastQC v0.11.5. STAR (version 2.6.0c) was used to compare and align the sequencing reads to the Human reference genome (Homo_sapiens. GRCh38.91). The reads were counted using Feature Count and the RPKM (Reads Per Kilobase of transcript, per Million mapped reads) values were generated. Downstream analysis was done using R package EdgeR (version 3.14). Negative binomial distributions were used to calculate biological and technical variability. Differential gene expression was determined using Fisher exact test. False discovery rate (FDR), was controlled using the Benjamini-Hochberg procedure, and a cut-off criterion of FDR<0.05 was applied to identify differentially expressed genes. Differentially expressed genes were selected based on fold-change (>=|2|), and FDR value (q<0.05). PCA (Principal component analysis) plots and Volcano plot based on (−log p value) vs (Log fold change), was created using ggplot2 in R. ComplexHeatmap v1.10.2 package of Bioconductor was used to perform hierarchical clustering and generate Heatmap.

### Example 2—In Vivo Maturation of iPSC-Derived TEPs

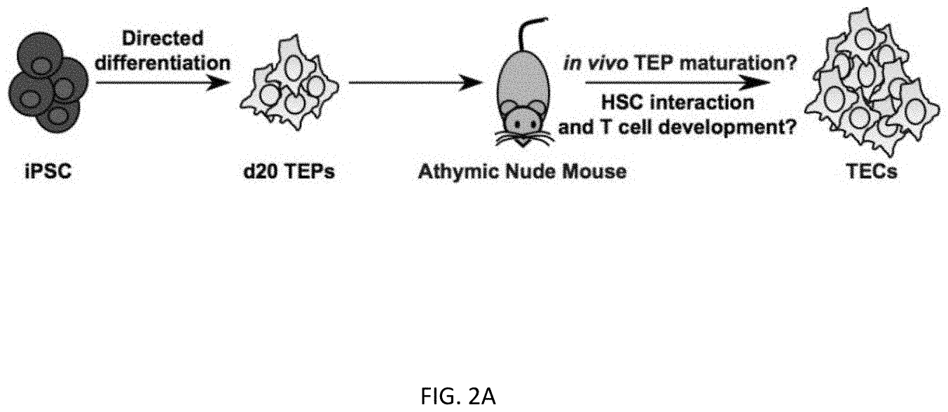

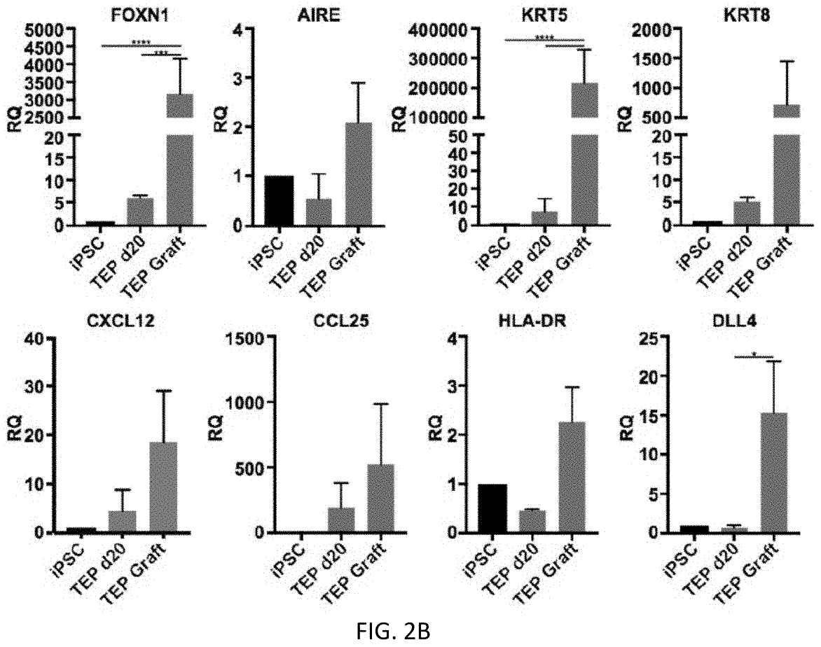

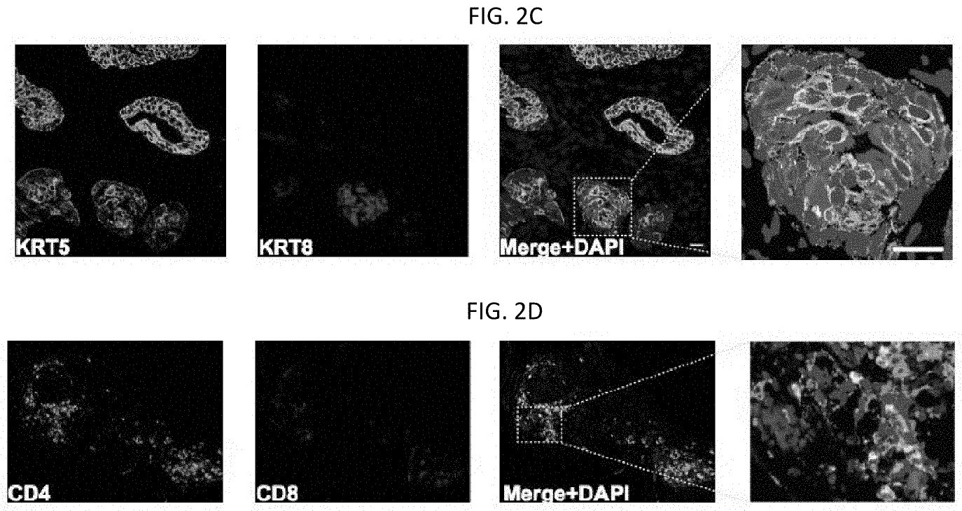

It is known that TEPs require interactions with hematopoietic stem and progenitor cells (HSPCs) to mature into functional thymic epithelial cells (TECs). Thus, to assess the ability of iPSC-derived TEPs to mature into functional TECs, d20 TEPs differentiated using condition 4, above, were transplanted under the kidney capsule of athymic nude mice, as previously described (FIG. 2A). Mice were euthanized 14-16 weeks post transplantation, and graft-bearing kidneys were prepared for multiple downstream analysis. qPCR analysis of dissected thymic grafts in comparison to in vitro TEPs indicates a robust induction of mature TEC markers FOXN1, AIRE, cytokeratin (KRT) 5 and 8, cytokines CXC112 and CCL25, HLA-DR (MHC-II) and delta like canonical notch ligand 4 (DLL4) (FIG. 2B) suggesting further differentiation in vivo.

The disclosed methods and systems are useful in inducing expression of markers for mature TECs. In some embodiments, the expression of one or more of FOXN1, AIRE, KRT5, KRT8, CXC112, CCL25, MHC-II, and DLL4. In many embodiments, expression of the disclosed markers may be enhanced relative to d20 TEP cells or iPSCs, from about more than about 1-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 200-fold, 300-fold, 400-fold, 500-fold, or more and less than about 1000-fold, 600-fold, 500-fold, 400-fold, 300-fold, 200-fold, 100-fold, 50-fold, 40-fold, 30-fold, 20-fold, 10-fold, 9-fold, 8-fold, 7-fold, 6-fold, 5-fold, 4-fold, 3-fold, 2-fold, 1.5-fold, or 1-fold. In many cases, fold expression may be determined based on standard cell expressing that same marker. In some cases the standard cell may be an iPSC cell that has not undergone differentiation to a TEP cell.

Immunofluorescence staining of graft sections probing for KRTS and 8 show single and double positive cells indicative of developing and mature TECs (FIG. 2C). These data indicate that iPSC-derived TEPs have the ability to further differentiate into patient-specific TECs upon transplantation into an in-vivo environment. Furthermore, immunofluorescence analysis of graft sections probing for mouse CD4 and CD8 T-cells markers show single and double positive cells near a TEC like structure, indicative of developing mouse T-cells within the grafts (FIG. 2D). In many embodiments, the in-vivo environment may be xenograft, allograft, or autograft.

Bulk RNA-seq was performed on four differentiated samples from graft tissue (Graft 1-4) obtained from two different samples of day 20 TEP differentiated from two different iPSC lines (CB74 and NHDF2.2) used for transplantation, two TEP samples (TEP 1 and 2) obtained from TEP grafts removed from individual mice for each iPSC line, and two primary neonatal thymi samples (Thy. 1 and 2), which acted as controls. Principle component analysis based on differentially expressed genes shows that samples are located within three discreet clusters reflecting their respective origin (i.e. thymi samples cluster together, separately from TEP samples, which also cluster together), with one of the TEP graft samples (Graft 1) located in between the clusters (FIG. 3A). Similarly, dendrogram hierarchical clustering of whole genome expression data of all samples shows clustering of TEP samples, primary thymi and grafts, with one graft sample (Graft 1) clustering closer to the starting TEP population than the other three graft samples.

Differential gene expression analysis between the sample groups was also performed. Comparing grafts versus day 20 TEPs shows 1,299 and 1,245 significantly up and down regulated, respectively. Examining specific genes, TPP markers HOXA3 and EYA1, are significantly downregulated in the grafts (FIG. 3C). Mature TEC markers FOXN1, KRTS, TP63, and CBX4 are significantly upregulated in grafts, as compared to day 20 TEPs, indicating that transplantation of iPSC-derived TEPs into nude mice results in further differentiation into patient-specific TECs (FIG. 3C). Counterintuitively, mRNA transcript levels of the cortical thymic epithelial cell (cTEC) marker KRT8 were down regulated in grafts compared to TEPs. KRT8 is primarily expressed in cTECs, however, previous studies have shown that TEPs exhibit a cTEC like phenotype. In line with this observation, KRT8 is also significantly less expressed in primary thymi when compared to day 20 TEPs, indicating that this result may reflect normal expression changes upon human TEP to TEC differentiation (FIG. 3D). Probing global differential gene expression of primary human neonatal thymi versus day 20 TEPs revealed 932 and 656 genes significantly up and down regulated, respectively (FIG. 3C, D). As shown, specific TEC markers, FOXN1, KRT5, TP63, CBX4 and AIRE, show higher expression in primary thymi. TEP markers HOXA3 and EYA1, as well as KRT8, are expressed at lower levels in primary thymi compared to day 20 TEPs. Direct comparison of expression levels of thymi to grafts shows 2,435 and 1,737 genes significantly up and down regulated, respectively (FIG. 3E). However, key TEC markers FOXN1, KRT5, TP63, and CBX4 are present at similar levels in both groups (FIG. 3E). Another TEC marker gene, AIRE an important regulator of negative T-cell selection in the medulla, is expressed at lower levels in grafts. This result might reflect a previously unappreciated impairment of negative selection, considering the TCR-HLA mismatched interactions of developing mouse T-cells on differentiating human thymic tissue employing the xenogenic nude mouse model system.

### Example 3—iPSC-Derived TECs are Functional and Can Support Developing Mouse T-Cells In Vivo

To investigate whether iPSC-derived thymus grafts are functional and able to support development of mouse T-cells, spleens from engrafted mice and sham control mice were harvested for further analysis at the time of graft removal. Immunohistochemical staining of two control and four graft-bearing mouse spleen sections using mouse-specific antibodies against CD3 or CD45 shows the presence of T-cells at higher ratio in the thymic graft-bearing mice (FIG. 3F). of CD3/CD45 double positive T-cells within isolated splenocytes from three control and seven graft bearing mice by fluorescence activated cell sorting (FACS) confirmed these results (FIG. 3G). Of note, previous reports have shown the presence of mature T-cells in the spleen and lymph nodes of athymic nude mice, despite the absence of a functional thymus. Mouse T-cells educated by iPSC-derived TEPs are functional, as indicated by the upregulation of activation markers CD69 and CD25 in response to in vitro stimulation (FIG. 3H). In sum, these data indicate that iPSC-derived TEPs are able to develop into functional, patient-specific TECs that are transcriptionally similar to primary human neonatal thymi when transplanted in vivo. Furthermore, iPSC-derived TECs are able to support T-cell development in athymic nude mice.

### Example 4—Single-Cell RNA Sequencing Resolves Thymic Cell Types in Grafts and Human Primary Neonatal Thymus

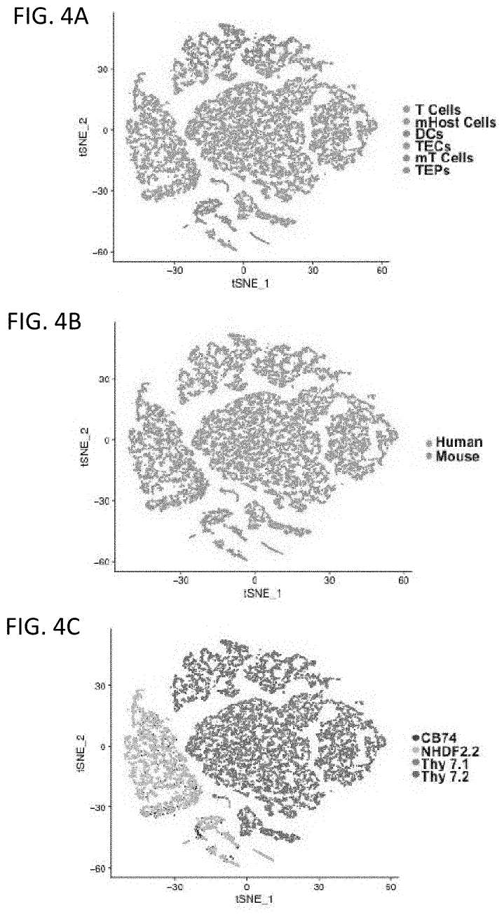

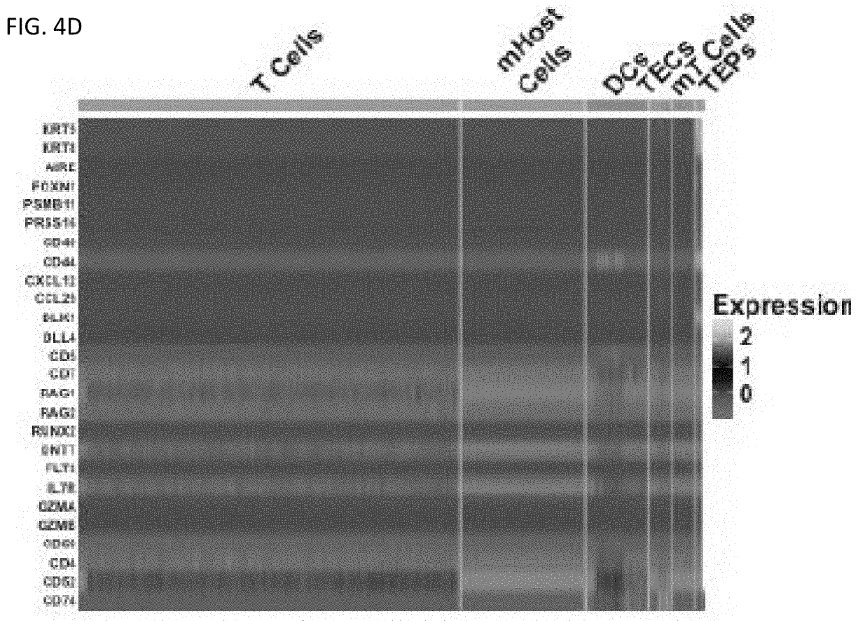

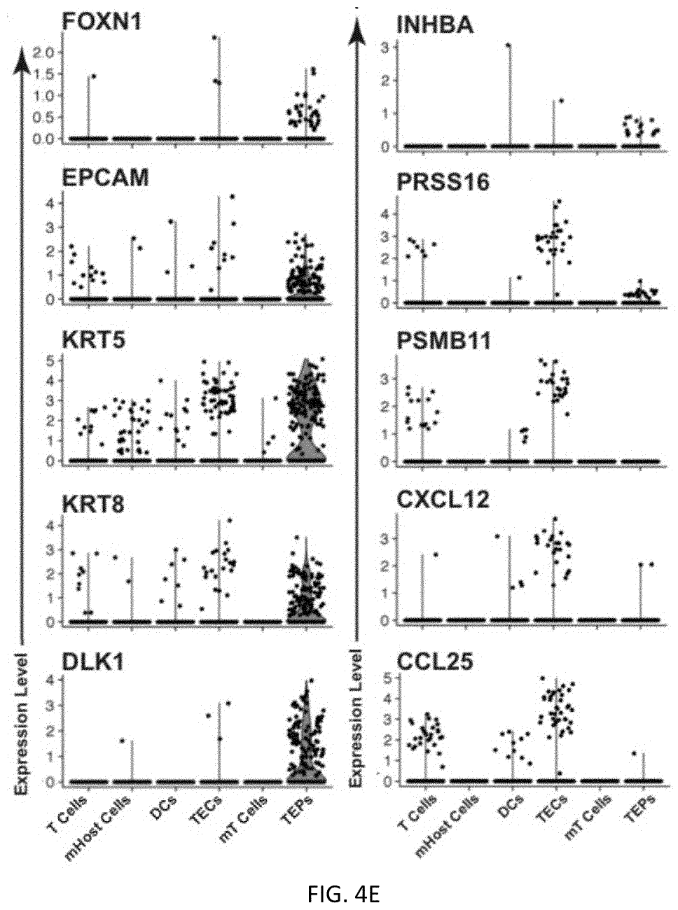

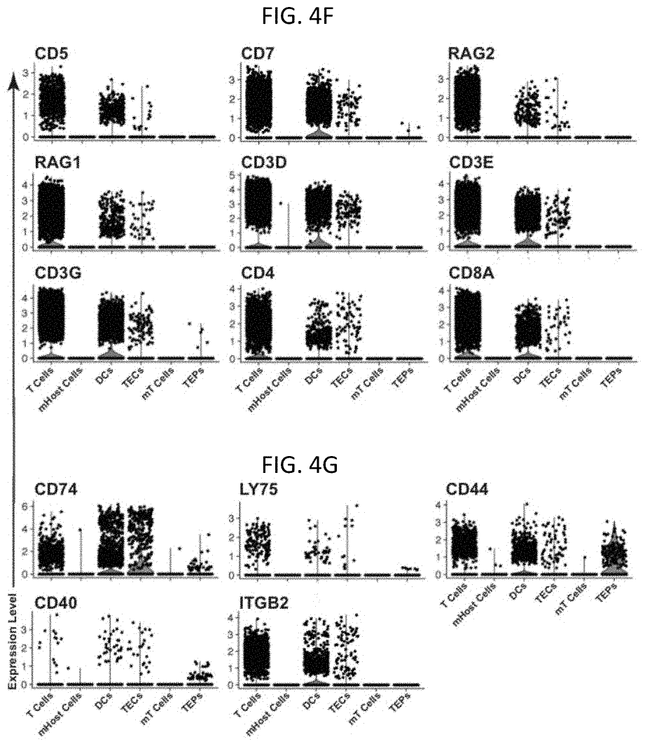

In addition to bulk RNA sequencing, grafts and human primary neonatal thymus were subjected to single-cell RNA sequencing analysis using 10× Genomics technology. The primary human neonatal thymus samples, referred to as Thy7.1 and Thy7.2, were derived from the same individual; however, single cell suspensions were prepared by two different methods to reduce digestion time that could potentially confound gene expression levels while simultaneously enriching for the TEC fraction in Thy7.2 (see Materials and Methods). While this approach did slightly increase the percentage of TECs detected by single-cell RNA sequencing from 2.50% in Thy7.1 to 4.58% in Thy7.2, T-cells still made up 79.24% of Thy7.2 single cell fraction, a small reduction from 87.27% in Thy7.1 (data not shown). To resolve different cell types in TEP graft and human primary neonatal thymus samples, unsupervised machine learning, including t-stochastic neighbor embedding (tSNE), was employed. This methodology effectively separates individual cells by tissue type and species (FIG. 4A, B), with 13 distinct clusters based on a Louvain algorithm variant (data not shown). Using a comprehensive, annotated list of marker genes, we further combined the 13 original clusters into 6 cell type-specific clusters consisting of human T-cells, dendritic cells (DCs), TECs, and TEPs and mouse T-cells and other host cells (FIG. 4A, D). Individual human and mouse cells could be successfully identified using the Cell Ranger (10× Genomics) pipeline for human and mouse datasets, verifying the 6 cell type specific clusters based on their species (FIG. 4B). As expected, tSNE analysis by species shows mouse derived cells present only in the TEP graft samples, with no mouse cells identified in the human primary neonatal thymus samples (FIG. 4B, C). Notably, TEP graft derived cells cluster with primary thymi cells in the TEP/TEC clusters, indicating that transplantation of iPSC-derived TEPs in vivo results in the generation of patient-specific TECs that resemble bona fide primary TECs (FIG. 4C).

Next, violin plots were used to visualize the expression patterns of individual genes in our single cell data sets and verify the validity of cell type specific clustering. Thymic specific markers FOXN1, EPCAM, KRT5, and KRT8 are most highly expressed in the TEP and TEC clusters, as compared to all other cell types (FIG. 4E). Key cortical TEC (cTEC) markers, PRSS16 and PSMB11, are also specifically expressed in the TEC cluster (FIG. 4E). LY75 (CD205) is known to be present in TEPs and TECs, and is expressed, albeit at low levels, in the TEP and TEC clusters (FIG. 4G). Additionally, key cytokines, CXCL12 and CC125, known to be expressed by thymic cells to attract hematopoietic stem cells, are expressed by cells of the TEC cluster (FIG. 4E). Activin A has recently been implicated in the induction of TEP differentiation towards TECs. Indeed, Inhibin beta A (INHBA), a subunit of activin A, is expressed specifically in the TEP cluster, albeit at low levels (FIG. 4E), indicating that Activin A may also play a role in human TEC development. NOTCH signaling is critical for T-cell commitment and development; in line with this notion delta like non-canonical Notch ligand 1 (DLK1) is found to be highly expressed only in the TEP population (FIG. 4E).

Within the T-cell compartment, key markers of developing T-cells are detected, such as progenitor and developing T-cell markers CD5 and CD7, RAG1 and 2, and CD3, 4, and 8 (FIG. 4F). Lastly, dendritic cell markers, some of which are known to be also expressed by TECs, are strongly expressed in the T-cell, DC, and TEC compartments (FIG. 4G). The present analysis provides novel insights into expression patterns and their changes in distinct human thymic cell types, both stem cell derived and neonatal. All scRNAseq data has been hosted on the Russ lab server (www.russlab.com/scRNA) using UCSC Cell Browser (cellbrowser.readthedocs.io)

### Example 5—iPSC-Derived TECs Cluster with Primary Neonatal Human Thymus TECs

To further resolve the cell types within the TEP/TEC clusters, we subset and re-analyzed the TEP and TEC clusters together (FIG. 5A, B). tSNE analysis identified 9 clusters within the combined TEP and TEC cell population (FIG. 5B). Sample specific tSNE analysis shows the distribution of TEP/TEC cells throughout the newly generated clusters (FIG. 5C). Importantly, iPSC-derived TECs cluster together with primary thymus TECs in clusters 0 and 4, while clusters 1, 3, 7, and 8 are comprised exclusively of iPSC-derived TEPs or TECs, suggesting that these clusters may contain developing thymic cells not readily present in the postnatal primary thymus sample (FIG. 5B, C). Co-expression of key markers FOXN1, KRT5, KRT8, and DLK1, in a large number of cells in cluster 3 indicates that the majority of TEPs are contained in this subset (FIG. 5F). TEC markers PSMB11, PRSS16, and CCL25, as well as individual expression of KRT5 or KRT8 are present in cells of cluster 5 at moderate to high levels, indicating a mature TEC population (FIG. 5G). KRT5 is expressed by more cells, and at slightly higher levels in cluster 5 than in cluster 7, however, a very low number of cells in cluster 5 also express autoimmune regulator (AIRE) (FIG. 5G). As medullary TECs (mTECs) are marked by the expression of both KRT5 and AIRE11, the data suggests that cluster 5 may represent mTECs. However, cTEC markers PSMB11 and PRSS16 are also predominately expressed in cluster 5, with cTEC distinguishing KRT8 being expressed predominately in cluster 7 (FIG. 5G). By distinguishing between unspliced and spliced mRNAs in single-cell RNA-seq data, RNA velocity can be used to predict the likely future state of any individual cell within the data set. RNA velocity analysis predicts the directionality of TEP graft derived cells as moving from cluster 1 and 8, towards clusters 3 and 7 (FIG. 5B,5D). Monocle's pseudo-time analysis was applied to the re-clustered TEP/TEC clusters, and one branch point was identified (FIG. 5E, 5F). Our detailed analysis shows that some iPSC-derived TECS and primary TECs exhibit overlapping expression profiles rendering them indistinguishable by the bioinformatic approaches employed, suggesting a bona fide TEC phenotype. However, iPSC-derived TEPs still differentiating into TECs appear to follow two differential developmental trajectories, only one of which leads towards primary TECs. Without wishing to be limited by theory, this could be a result of the xenograft interaction of iPSC-derived TEPs with developing mouse T-cells.

### Example 6—Mouse T-Cells Develop Within iPSC-Derived Thymic Tissue

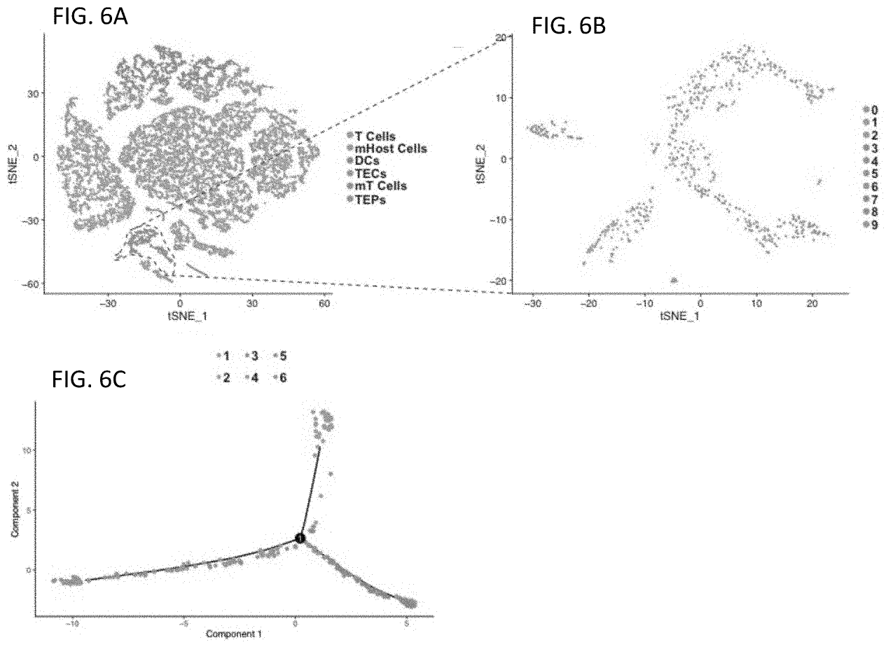

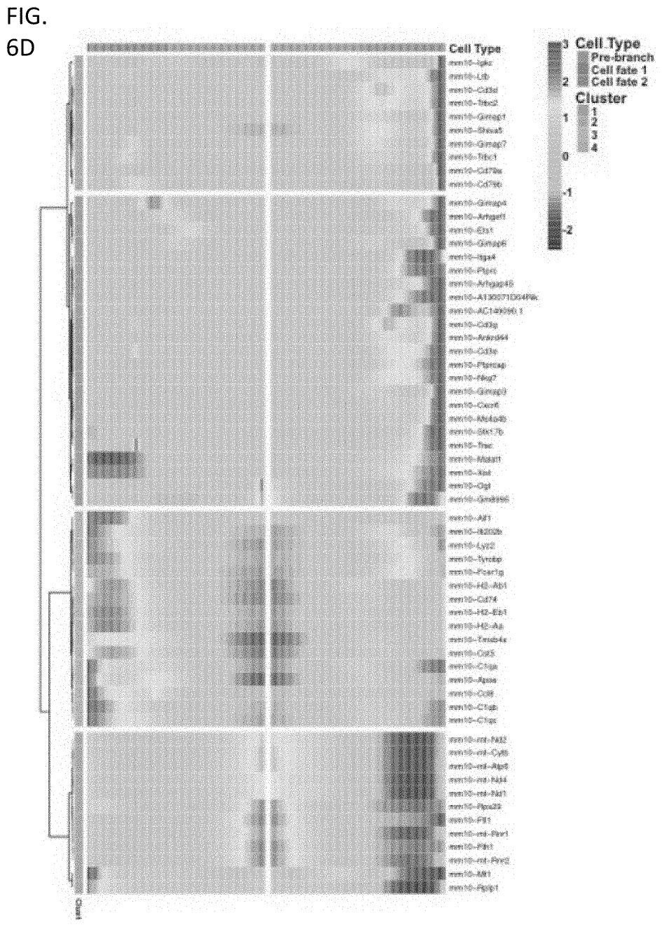

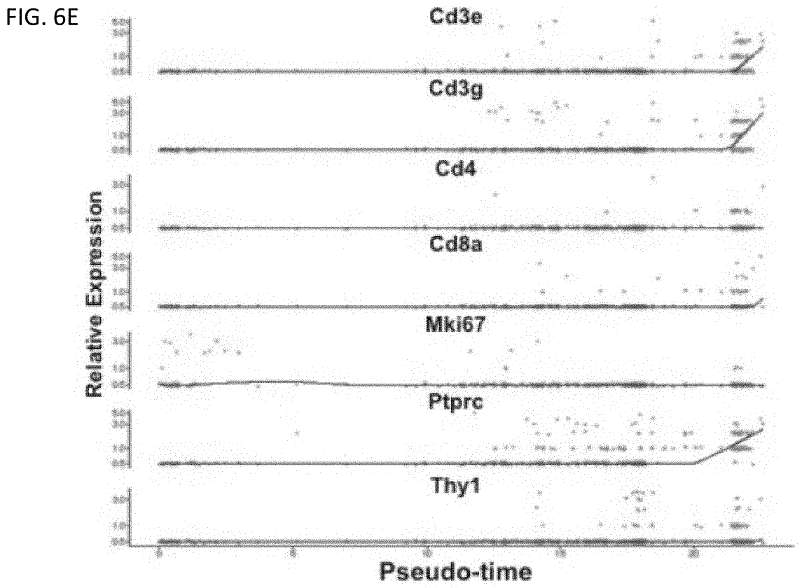

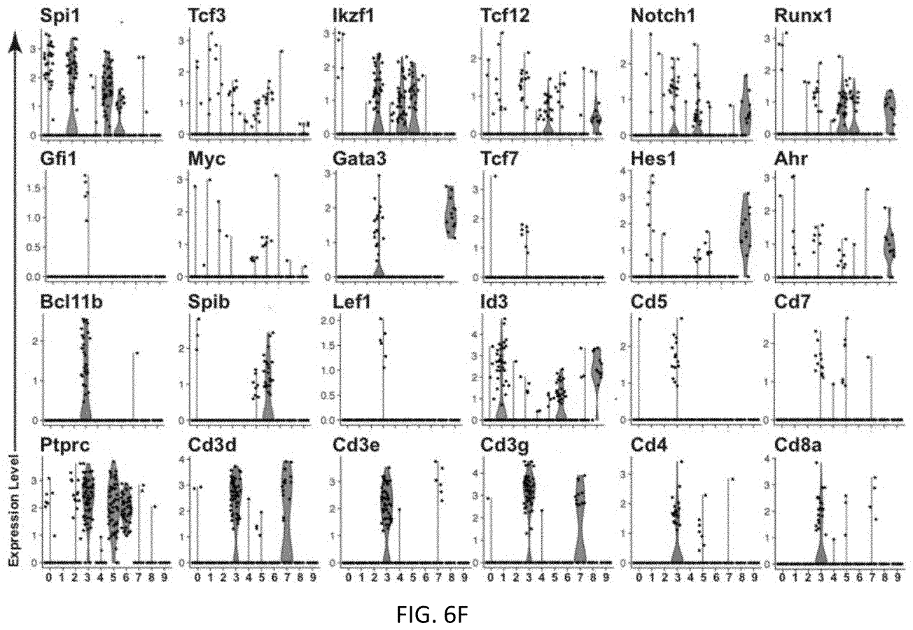

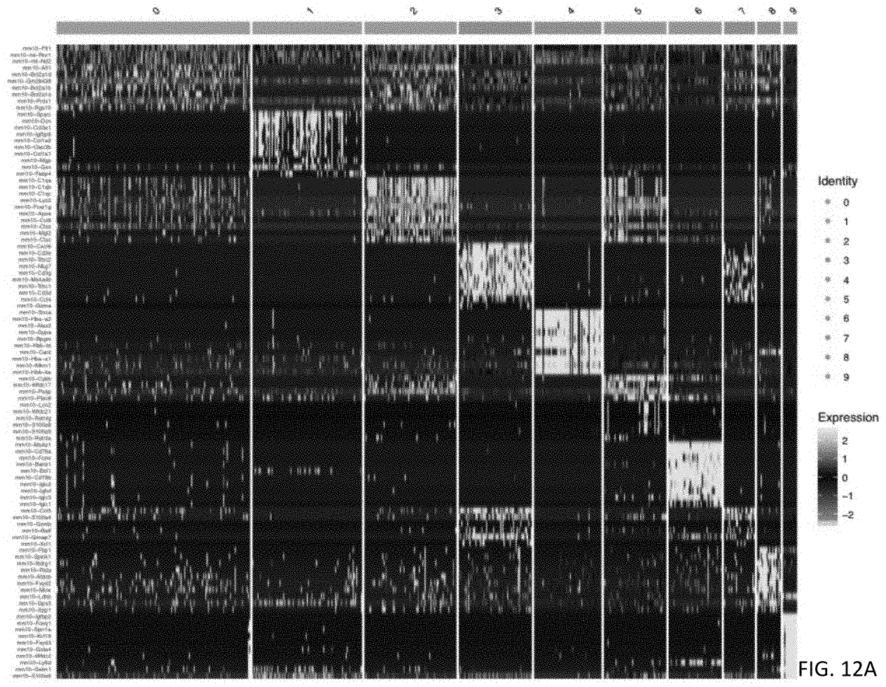

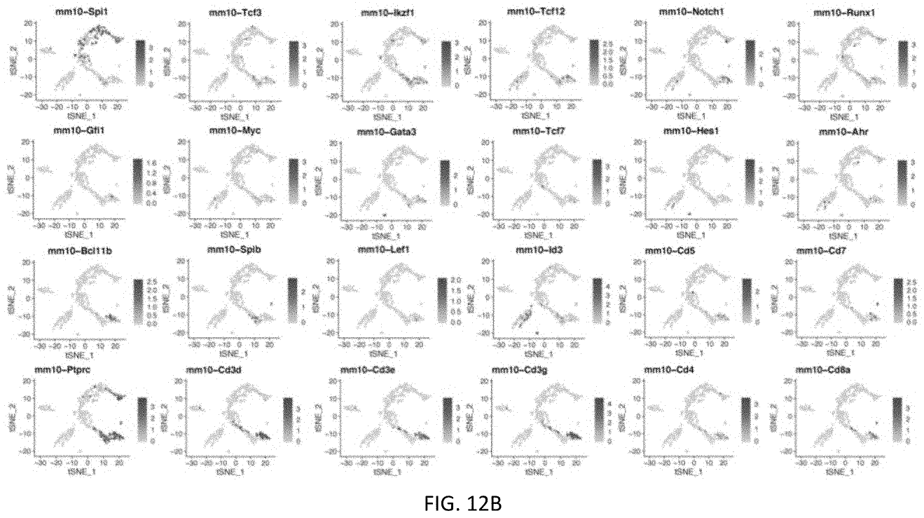

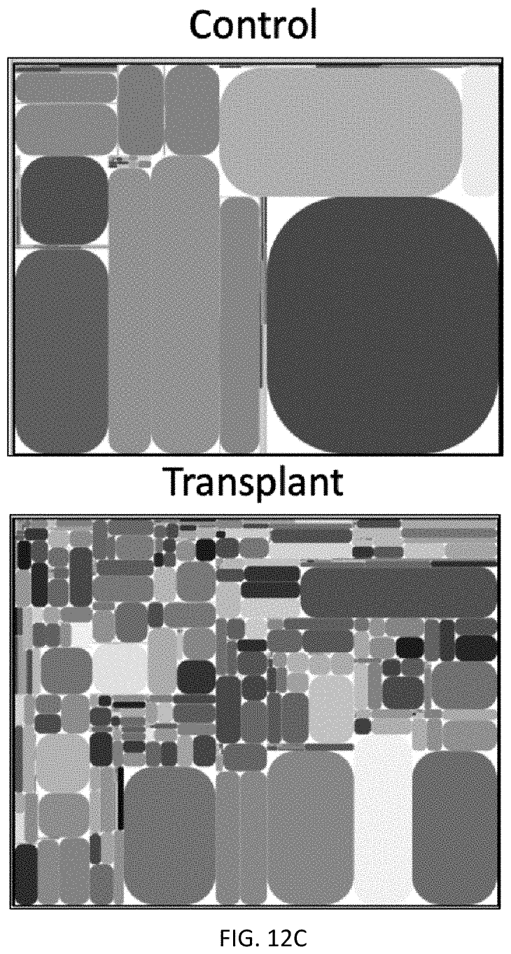

To determine whether de novo T-cell development was indeed occurring in iPSC-derived thymic grafts as suggested by our previous analysis (FIG. 3F-H), we subset and re-analyzed the mouse T-cell cluster individually (mT-cells) (FIG. 6A, B). tSNE analysis identified 10 clusters within the mT-cell population (FIG. 6B). Indeed, key transcription factors involved in mouse T-cell development are present in many of the T-cell sub clusters, indicating the developmental progression of mouse T-cells in nude mice that received a TEP graft (FIG. 6F, FIG. 12B). RNA velocity analysis shows many cells moving towards cluster 3, which contains the highest number of cells expressing later markers of T-cell differentiation, such as Cd3, 4, and 8 (FIG. 12A, FIG. 6F, FIG. 12B), indicating the directionality of mouse T-cell development in the graft tissue. Pseudo time analysis shows only one branch point, with cells developing towards two cell fates (FIG. 6C,D). Gene-specific pseudo time analysis shows 3 developmental states (FIG. 6E). Key T-cell markers, Cd3e, Cd3g, Cd4, Cd8a, and Ptprc (Cd45) show low expression during states 1 and 2, but increases during state 3, indicative of the developmental progression of the mouse derived T-cells in the TEP graft (FIG. 6E). Additionally, we observe a bump in the proliferation marker Mki67 during stage 1, indicative of cell proliferation that is associated with T-cell development (FIG. 6E). During the course of development, cells branches out in different trajectories based on their developmental lineage. The branch points heatmap was made using the Monocle, which shows genes that are enriched at the branch point, as well as at each cell fate. In cell fate 2, we observe the enrichment of innate immune cell markers (FIG. 6D). Further, cell fate 1 shows enrichment for markers of T-cell development, such as Aifl and Tyrobp (FIG. 6D). Interestingly, cluster 4 cell fate 2 shows enrichment for B cell markers, as B cells have also been shown to be present in the thymus (FIG. 6D).

T-cells in graft bearing mice show significantly enhanced diversity in T-Cell Receptor (TCR) sequence. FIG. 12C shows the clustering of TCR sequences in control versus grafted mice. TCR sequencing was conducted on CD45 and CD3 double positive T-cells that were FACS isolated from the blood of either control or graft bearing nude mice. TCR sequencing and analysis was performed using iRepertoir technology. Each color bubble shown in FIG. 12C corresponds to an individual TCR sequence identified, while the size of each bubble indicates the abundance of that specific sequence, relative to all reads. Thus, many small TCR bubbles indicate higher diversity/heterogeneity.

The above specification, examples, and data provide a description of the features and uses of some exemplary implementations of Applicants inventive subject matter. Many implementations of Applicant's subject matter may be made without departing from its spirit and scope. Furthermore, other features of the different implementations may be combined in yet further implementations without departing from the recited claims.

What is claimed is:

1. A method for generating patient-specific thymic epithelial cells (TECs), the method comprising: isolating a cell from the patient;
administering one or more factors to the cell to reprogram the cell and create an induced pluripotent stem cell (iPSC);
culturing the patient-specific iPSC for 9-14 days in a differentiation media to create a thymic epithelial progenitor (TEP) cell; and
transferring at least one TEP into a recipient; and
allowing the TEP cell to differentiate into a TEC.

2. The method of claim 1, wherein the iPSC is derived from a hematopoietic stem cell (HSC) or peripheral blood mononuclear cell (PBMC).

3. The method of claim 1, wherein the TECs are mature, functional, patient-specific thymic epithelial cells (TEC).

4. The method of claim 1, further comprising the step of contacting a patient-derived T-cell with the TEC to produce a functional T-cell.

5. The method of claim 1, further comprising the step of contacting a patient-derived T-cell with the TEPs to produce a functional T-cell and or functional TECs.

6. The method of any of claims 4-5, wherein the mature T-cell expresses one or more of CD69, CD25, CD5, CD7, CD4, CD8, CD3, CD45, RAG1, and RAG2.

7. The method of any of claims 4-6, wherein the number of peripheral T-cells is greater than in a recipient that did not receive a patient-specific TEP.

8. The method of claim 1, wherein the differentiation media comprises one or more pathway activators and/or pathway inhibitors.

9. The method of claim 8, wherein the activated and/or inhibited pathway is one or more of Activin, WNT, BMP, RA, TGFβ, SHH, and FGFβ.

10. The method of claim 9, wherein the inhibited pathways comprise one or more of SHH, and TGFβ.

11. The method of claim 9, wherein the one or more activated pathways are activated by at least one of Activin A, WNT3a, BMP4, SAG, TTNPB, and FGF8b.

12. The method of claim 10, wherein the one or more inhibited pathways are inhibited by at least one of Ly-364947 and Sant1.

13. The method of claim 1, wherein the TEC cells express one or markers selected from FOXN1, AIRE, CK5, CK8, CXCL12, CCL25, DLL4, and HLA-DR.

14. The method of claim 1, wherein the differentiation media comprises one or more of Activin A, Wnt3a, TTNPB, BMP4, LY364947, FGF8b, FGF8a, SAG, and SANT-1 on days 0-13.

15. The method of claim 1, further comprising:
co-culturing the TEP cells with hematopoietic stem and progenitor cells (HPSCs) or HSCs for about 7 days generates TECs.

16. The method of claim 15, wherein the TECs express one or more genetic markers selected from FOXN1, AIRE, CK5, CK8, CXCL12, CCL25, DLL4, TP63, CBX4, and HLA-DR.

17. The method of claim 15, wherein the TECs express markers typically expressed by cortical TECs and medullary TECs.

18. The method of claim 17, wherein the markers are selected from KRT5, KRT8, AIRE, PSMB11, and PRSS16.

19. A population of differentiated, mature thymic epithelial cells comprising:
one or more thymic epithelial cells (TECs) expressing one or more of KRT5, KRT8, AIRE, PSMB11, and PRSS16, wherein the one or more TECs is derived from thymic epithelial progenitor (TEP) cell derived from an induced pluripotent stem cell (iPSC) grown in-vitro in the presence of one or more of Activin A, Wnt3a, TTNPB, BMP4, LY364947, FGF8b, FGF8a, SAG, and SANT-1, and wherein the TEP differentiates into a TEC in-vivo after transplantation into a recipient.

20. The population of differentiated, mature thymic epithelial cells of claim 19, wherein the iPSCs are grown in-vitro for between 12 and 14 days.

21. The population of differentiated, mature thymic epithelial cells of claim 19 or claim 20, wherein the iPSCs are derived from one or more cells of the recipient.

22. The population of differentiated, mature thymic epithelial cells any of claims 19-21, wherein the TECs express one or more markers selected from FOXN1, AIRE, CK5, CK8, CXCL12, CCL25, DLL4, and HLA-DR.

23. A system for generating a mature functional thymic epithelial cell comprising:
a method for inducing a pluripotent stem cell from a cell of a subject;
a culture device for growing the induced pluripotent stem cell for 12-14 days in the presence and absence of one or more of Activin A, Wnt3a, TTNPB, BMP4, LY364947, FGF8b, FGF8a, SAG, and SANT-1 to produce a differentiated thymic epithelial progenitor cell;
a device for implanting one or more thymic epithelial progenitor cells into a subject.

24. Use of a population of cells, as described in any of claims 19-22, or a cell produced by the method of any of claims 1-18, in the preparation of a medicament for the treatment of an immune condition or disorder, wherein the disorder or condition is selected from a non-existent thymus, damaged thymus, dysfunctional thymus, diseased thymus, aged thymus, diabetes Type 1, auto-immune, allorejection, cancer, and combinations thereof.

25. A method of treating a subject suffering from or at risk of an immune condition or disorder, comprising:
administering to the subject a TEP cell according to any of claims 19-22, or a cell produced by the method of any of claims 1-18; wherein the disorder or condition is selected from a non-existent thymus, damaged thymus, dysfunctional thymus, diseased thymus, aged thymus, Type 1 diabetes, auto-immune, allorejection, cancer, and combinations thereof.

26. A method of treating a patient with a thymic disorder comprising:
administering one or more thymic epithelial progenitor (TEP) cells derived from a patient specific induced pluripotent stem cell (iPSC) grown in-vitro in the presence of one or more of Activin A, Wnt3a, TTNPB, BMP4, LY364947, FGF8b, FGF8a, SAG, and SANT-1, and wherein the TEP differentiates into a thymic epithelial cells (TECs) in-vivo after administration to the patient, wherein the iPSCs are grown in-vitro for between 12 and 14 days, and wherein the mature TECs express one or more markers selected from FOXN1, AIRE, CK5, CK8, CXCL12, CCL25, DLL4, and HLA-DR.

27. The method of claim 26, wherein the iPSCs are derived from one or more of the patient's skin, uterine tissue, kidney, liver, muscle, adrenal glands, blood.

28. The method of claim 26 or 27, wherein the selected marker is expressed at between 0.5-fold and 1000-fold in mature iPSC-derived TECs relative to the administered TEP cells or the iPSCs.


SEQUENCE LISTING (41 sequences)

SEQ ID NO: 1 - DNA, 1812 bp, Artificial Sequence
  Homo sapiens actin beta (ACTB) nucleotide
accgccgagaccgcgtccgccccgcgagcacagagcctcgcctttgccgatccgccgcccgtccacacccgccgccagctcaccatggatgatgatatcgccgcgctcgtcgtcgacaacggctccggcatgtgcaaggccggcttcgcgggcgacgatgccccccgggccgtcttcccctccatcgtggggcgccccaggcaccagggcgtgatggtgggcatgggtcagaaggattcctatgtgggcgacgaggcccagagcaagagaggcatcctcaccctgaagtaccccatcgagcacggcatcgtcaccaactgggacgacatggagaaaatctggcaccacaccttctacaatgagctgcgtgtggctcccgaggagcaccccgtgctgctgaccgaggcccccctgaaccccaaggccaaccgcgagaagatgacccagatcatgtttgagaccttcaacaccccagccatgtacgttgctatccaggctgtgctatccctgtacgcctctggccgtaccactggcatcgtgatggactccggtgacggggtcacccacactgtgcccatctacgaggggtatgccctcccccatgccatcctgcgtctggacctggctggccgggacctgactgactacctcatgaagatcctcaccgagcgcggctacagcttcaccaccacggccgagcgggaaatcgtgcgtgacattaaggagaagctgtgctacgtcgccctggacttcgagcaagagatggccacggctgcttccagctcctccctggagaagagctacgagctgcctgacggccaggtcatcaccattggcaatgagcggttccgctgccctgaggcactcttccagccttccttcctgggcatggagtcctgtggcatccacgaaactaccttcaactccatcatgaagtgtgacgtggacatccgcaaagacctgtacgccaacacagtgctgtctggcggcaccaccatgtaccctggcattgccgacaggatgcagaaggagatcactgccctggcacccagcacaatgaagatcaagatcattgctcctcctgagcgcaagtactccgtgtggatcggcggctccatcctggcctcgctgtccaccttccagcagatgtggatcagcaagcaggagtatgacgagtccggcccctccatcgtccaccgcaaatgcttctaggcggactatgacttagttgcgttacaccctttcttgacaaaacctaacttgcgcagaaaacaagatgagattggcatggctttatttgttttttttgttttgttttggttttttttttttttttggcttgactcaggatttaaaaactggaacggtgaaggtgacagcagtcggttggagcgagcatcccccaaagttcacaatgtggccgaggactttgattgcacattgttgtttttttaatagtcattccaaatatgagatgcgttgttacaggaagtcccttgccatcctaaaagccaccccacttctctctaaggagaatggcccagtcctctcccaagtccacacaggggaggtgatagcattgctttcgtgtaaattatgtaatgcaaaatttttttaatcttcgccttaatacttttttattttgttttattttgaatgatgagccttcgtgcccccccttcccccttttttgtcccccaacttgagatgtatgaaggcttttggtctccctgggagtgggtggaggcagccagggcttacctgtacactgacttgagaccagttgaataaaagtgcacaccttaaaaatga

SEQ ID NO: 2 - protein, 375 aa, Artificial Sequence
  Homo sapiens Actin Beta (ACTB) protein
  MISC_FEATURE (1)..(375): ACTB Protein
MDDDIAALVVDNGSGMCKAGFAGDDAPRAVFPSIVGRPRHQGVMVGMGQKDSYVGDEAQSKRGILTLKYPIEHGIVTNWDDMEKIWHHTFYNELRVAPEEHPVLLTEAPLNPKANREKMTQIMFETFNTPAMYVAIQAVLSLYASGRTTGIVMDSGDGVTHTVPIYEGYALPHAILRLDLAGRDLTDYLMKILTERGYSFTTTAEREIVRDIKEKLCYVALDFEQEMATAASSSSLEKSYELPDGQVITIGNERFRCPEALFQPSFLGMESCGIHETTFNSIMKCDVDIRKDLYANTVLSGGTTMYPGIADRMQKEITALAPSTMKIKIIAPPERKYSVWIGGSILASLSTFQQMWISKQEYDESGPSIVHRKCF

SEQ ID NO: 3 - DNA, 2690 bp, Artificial Sequence
  Homo sapiens autoimmune regulator (AIRE) transcript - (AIRE nucleotide)
ggggagacgggcgggcgcacagccggcgcggaggccccacagccccgccgggacccgaggccaagcgaggggctgccagtgtcccgggacccaccgcgtccgccccagccccgggtccccgcgcccaccccatggcgacggacgcggcgctacgccggcttctgaggctgcaccgcacggagatcgcggtggccgtggacagcgccttcccactgctgcacgcgctggctgaccacgacgtggtccccgaggacaagtttcaggagacgcttcatctgaaggaaaaggagggctgcccccaggccttccacgccctcctgtcctggctgctgacccaggactccacagccatcctggacttctggagggtgctgttcaaggactacaacctggagcgctatggccggctgcagcccatcctggacagcttccccaaagatgtggacctcagccagccccggaaggggaggaagcccccggccgtccccaaggctttggtaccgccacccagactccccaccaagaggaaggcctcagaagaggctcgagctgccgcgccagcagccctgactccaaggggcaccgccagcccaggctctcaactgaaggccaagccccccaagaagccggagagcagcgcagagcagcagcgccttccactcgggaacgggattcagaccatgtcagcttcagtccagagagctgtggccatgtcctccggggacgtcccgggagcccgaggggccgtggaggggatcctcatccagcaggtgtttgagtcaggcggctccaagaagtgcatccaggttggcggggagttctacactcccagcaagttcgaagactccggcagtgggaagaacaaggcccgcagcagcagtggcccgaagcctctggttcgagccaagggagcccagggcgctgcccccggtggaggtgaggctaggctgggccagcagggcagcgttcccgcccctctggccctccccagtgacccccagctccaccagaagaatgaggacgagtgtgccgtgtgtcgggacggcggggagctcatctgctgtgacggctgccctcgggccttccacctggcctgcctgtcccctccgctccgggagatccccagtgggacctggaggtgctccagctgcctgcaggcaacagtccaggaggtgcagccccgggcagaggagccccggccccaggagccacccgtggagaccccgctccccccggggcttaggtcggcgggagaggaggtaagaggtccacctggggaacccctagccggcatggacacgactcttgtctacaagcacctgccggctccgccttctgcagccccgctgccagggctggactcctcggccctgcaccccctactgtgtgtgggtcctgagggtcagcagaacctggctcctggtgcgcgttgcggggtgtgcggagatggtacggacgtgctgcggtgtactcactgcgccgctgccttccactggcgctgccacttcccagccggcacctcccggcccgggacgggcctgcgctgcagatcctgctcaggagacgtgaccccagcccctgtggagggggtgctggcccccagccccgcccgcctggcccctgggcctgccaaggatgacactgccagtcacgagcccgctctgcacagggatgacctggagtcccttctgagcgagcacaccttcgatggcatcctgcagtgggccatccagagcatggcccgtccggcggcccccttcccctcctgaccccagatggccgggacatgcagctctgatgagagagtgctgagaaggacacctccttcctcagtcctggaagccggccggctgggatcaagaaggggacagcgccacctcttgtcagtgctcggctgtaaacagctctgtgtttctggggacaccagccatcatgtgcctggaaattaaaccctgccccacttctctactctggaagtccccgggagcctctccttgcctggtgacctactaaaaatataaaaattagctgggtgtggtggtgggtgcctgtaatcccagctacatgggagcctgaggcatgagaatcacttgaactcgggaggtggaggttgcagtgagctgagattgcgccactgcactccagtctggtcggcaagagtgagactccgtctcaaaaacaaaacaaaacaaaaaaaccacataacataaatttatcatctcgaccacttttcagttcagtggcattcacatctcatgtaaccattgccaccaccatctgcagagctgttccagcttcctgaatggaaactctggccccatgaaacactcactccccattcccctcccaacccctggcaccctccactctactttctgtctctatgacttctccagggacctcatggaagtggaatcacacagtatctgtccttttgtgtctgtcttattttactcagcatggtggccccaaggttcatccatgttgtagcatgtgccgaaatctgcttccttttttaaggctgagtaatattccagcacatggatggactgcattgtgttcatctatgctttcctcagcagacacctgggtgcttccaccttttggctgttgtgagtaatgctgctatgaatatgtgtgtgcaaatatctgtgtgagtctttgagtaaaatcgctggatcacatggtaa

SEQ ID NO: 4 - protein, 545 aa, Artificial Sequence
  Homo sapiens autoimmune regulator (AIRE) - (AIRE protein)
MATDAALRRLLRLHRTEIAVAVDSAFPLLHALADHDVVPEDKFQETLHLKEKEGCPQAFHALLSWLLTQDSTAILDFWRVLFKDYNLERYGRLQPILDSFPKDVDLSQPRKGRKPPAVPKALVPPPRLPTKRKASEEARAAAPAALTPRGTASPGSQLKAKPPKKPESSAEQQRLPLGNGIQTMSASVQRAVAMSSGDVPGARGAVEGILIQQVFESGGSKKCIQVGGEFYTPSKFEDSGSGKNKARSSSGPKPLVRAKGAQGAAPGGGEARLGQQGSVPAPLALPSDPQLHQKNEDECAVCRDGGELICCDGCPRAFHLACLSPPLREIPSGTWRCSSCLQATVQEVQPRAEEPRPQEPPVETPLPPGLRSAGEEVRGPPGEPLAGMDTTLVYKHLPAPPSAAPLPGLDSSALHPLLCVGPEGQQNLAPGARCGVCGDGTDVLRCTHCAAAFHWRCHFPAGTSRPGTGLRCRSCSGDVTPAPVEGVLAPSPARLAPGPAKDDTASHEPALHRDDLESLLSEHTFDGILQWAIQSMARPAAPFPS

SEQ ID NO: 5 - DNA, 991 bp, Artificial Sequence
  Homo sapiens C-C motif chemokine ligand 25 (CCL25), transcript variant 1 - (CCL25 nucleotide)
gcagatgggacagcttggcctacagcccggcgggcatcagctcccttgacccagtggatatcggtggccccgttattcgtccaggtgcccagggaggaggacccgcctgcagcatgaacctgtggctcctggcctgcctggtggccggcttcctgggagcctgggcccccgctgtccacacccaaggtgtctttgaggactgctgcctggcctaccactaccccattgggtgggctgtgctccggcgcgcctggacttaccggatccaggaggtgagcgggagctgcaatctgcctgctgcgatattctacctccccaagagacacaggaaggtgtgtgggaaccccaaaagcagggaggtgcagagagccatgaagctcctggatgctcgaaataaggtttttgcaaagctccaccacaacacgcagaccttccaagcaggccctcatgctgtaaagaagttgagttctggaaactccaagttatcatcgtccaagtttagcaatcccatcagcagcagtaagaggaatgtctccctcctgatatcagctaattcaggactgtgagccggctcatttctgggctccatcggcacaggaggggccggatctttctccgataaaaccgtcgccctacagacccagctgtccccacgcctctgtcttttgggtcaagtcttaatccctgcacctgagttggtcctccctctgcacccccaccacctcctgcccgtctggcaactggaaagagggagttggcctgattttaagccttttgccgctccggggaccagcagcaatcctgggcagccagtggctcttgtagagaagacttaggatacctctctcactttctgtttcttgccgtccaccccgggccatgccagtgtgtccctctgggtccctccaaaactctggtcagttcaaggatgcccctcccaggctatgcttttctataacttttaaataaaccttggggggtgatggagtca

SEQ ID NO: 6 - protein, 150 aa, Artificial Sequence
  C-C motif chemokine 25 isoform 1 precursor [Homo sapiens] - (CCL25 protein)
MNLWLLACLVAGFLGAWAPAVHTQGVFEDCCLAYHYPIGWAVLRRAWTYRIQEVSGSCNLPAAIFYLPKRHRKVCGNPKSREVQRAMKLLDARNKVFAKLHHNTQTFQAGPHAVKKLSSGNSKLSSSKFSNPISSSKRNVSLLISANSGL

SEQ ID NO: 7 - DNA, 1928 bp, Artificial Sequence
  Homo sapiens C-X-C motif chemokine ligand 12 (CXCL12), transcript variant 1 - (CXCL12 nucleotide)
gccgcactttcactctccgtcagccgcattgcccgctcggcgtccggcccccgacccgcgctcgtccgcccgcccgcccgcccgcccgcgccatgaacgccaaggtcgtggtcgtgctggtcctcgtgctgaccgcgctctgcctcagcgacgggaagcccgtcagcctgagctacagatgcccatgccgattcttcgaaagccatgttgccagagccaacgtcaagcatctcaaaattctcaacactccaaactgtgcccttcagattgtagcccggctgaagaacaacaacagacaagtgtgcattgacccgaagctaaagtggattcaggagtacctggagaaagctttaaacaagtaagcacaacagccaaaaaggactttccgctagacccactcgaggaaaactaaaaccttgtgagagatgaaagggcaaagacgtgggggagggggccttaaccatgaggaccaggtgtgtgtgtggggtgggcacattgatctgggatcgggcctgaggtttgccagcatttagaccctgcatttatagcatacggtatgatattgcagcttatattcatccatgccctgtacctgtgcacgttggaacttttattactggggtttttctaagaaagaaattgtattatcaacagcattttcaagcagttagttccttcatgatcatcacaatcatcatcattctcattctcattttttaaatcaacgagtacttcaagatctgaatttggcttgtttggagcatctcctctgctcccctggggagtctgggcacagtcaggtggtggcttaacagggagctggaaaaagtgtcctttcttcagacactgaggctcccgcagcagcgcccctcccaagaggaaggcctctgtggcactcagataccgactggggctgggcgccgccactgccttcacctcctctttcaacctcagtgattggctctgtgggctccatgtagaagccactattactgggactgtgctcagagacccctctcccagctattcctactctctccccgactccgagagcatgcttaatcttgcttctgcttctcatttctgtagcctgatcagcgccgcaccagccgggaagagggtgattgctggggctcgtgccctgcatccctctcctcccagggcctgccccacagctcgggccctctgtgagatccgtctttggcctcctccagaatggagctggccctctcctggggatgtgtaatggtccccctgcttacccgcaaaagacaagtctttacagaatcaaatgcaattttaaatctgagagctcgctttgagtgactgggttttgtgattgcctctgaagcctatgtatgccatggaggcactaacaaactctgaggtttccgaaatcagaagcgaaaaaatcagtgaataaaccatcatcttgccactaccccctcctgaagccacagcagggtttcaggttccaatcagaactgttggcaaggtgacatttccatgcataaatgcgatccacagaaggtcctggtggtatttgtaactttttgcaaggcatttttttatatatatttttgtgcacatttttttttacgtttctttagaaaacaaatgtatttcaaaatatatttatagtcgaacaattcatatatttgaagtggagccatatgaatgtcagtagtttatacttctctattatctcaaactactggcaatttgtaaagaaatatatatgatatataaatgtgattgcagcttttcaatgttagccacagtgtattttttcacttgtactaaaattgtatcaaatgtgacattatatgcactagcaataaaatgctaattgtttcatggtataaacgtcctactgtatgtgggaatttatttacctgaaataaaattcattagttgttagtgatgga

SEQ ID NO: 8 - protein, 89 aa, Artificial Sequence
  stromal cell-derived factor 1 isoform alpha precursor [Homo sapiens] (CCXL12 expressed protein)
MNAKVVVVLVLVLTALCLSDGKPVSLSYRCPCRFFESHVARANVKHLKILNTPNCALQIVARLKNNNRQVCIDPKLKWIQEYLEKALNK

SEQ ID NO: 9 - DNA, 3426 bp, Artificial Sequence
  Homo sapiens delta like canonical Notch ligand 4 (DLL4) - transcript - (DLL4 nucleotide)
agtagcggcgctgcgcgcaggccgggaacacgaggccaagagccgcagccccagccgccttggtgcagcgtacaccggcactagcccgcttgcagccccaggattagacagaagacgcgtcctcggcgcggtcgccgcccagccgtagtcacctggattacctacagcggcagctgcagcggagccagcgagaaggccaaaggggagcagcgtcccgagaggagcgcctcttttcagggaccccgccggctggcggacgcgcgggaaagcggcgtcgcgaacagagccagattgagggcccgcgggtggagagagcgacgcccgaggggatggcggcagcgtcccggagcgcctctggctgggcgctactgctgctggtggcactttggcagcagcgcgcggccggctccggcgtcttccagctgcagctgcaggagttcatcaacgagcgcggcgtactggccagtgggcggccttgcgagcccggctgccggactttcttccgcgtctgccttaagcacttccaggcggtcgtctcgcccggaccctgcaccttcgggaccgtctccacgccggtattgggcaccaactccttcgctgtccgggacgacagtagcggcggggggcgcaaccctctccaactgcccttcaatttcacctggccgggtaccttctcgctcatcatcgaagcttggcacgcgccaggagacgacctgcggccagaggccttgccaccagatgcactcatcagcaagatcgccatccagggctccctagctgtgggtcagaactggttattggatgagcaaaccagcaccctcacaaggctgcgctactcttaccgggtcatctgcagtgacaactactatggagacaactgctcccgcctgtgcaagaagcgcaatgaccacttcggccactatgtgtgccagccagatggcaacttgtcctgcctgcccggttggactggggaatattgccaacagcctatctgtctttcgggctgtcatgaacagaatggctactgcagcaagccagcagagtgcctctgccgcccaggctggcagggccggctgtgtaacgaatgcatcccccacaatggctgtcgccacggcacctgcagcactccctggcaatgtacttgtgatgagggctggggaggcctgttttgtgaccaagatctcaactactgcacccaccactccccatgcaagaatggggcaacgtgctccaacagtgggcagcgaagctacacctgcacctgtcgcccaggctacactggtgtggactgtgagctggagctcagcgagtgtgacagcaacccctgtcgcaatggaggcagctgtaaggaccaggaggatggctaccactgcctgtgtcctccgggctactatggcctgcattgtgaacacagcaccttgagctgcgccgactccccctgcttcaatgggggctcctgccgggagcgcaaccagggggccaactatgcttgtgaatgtccccccaacttcaccggctccaactgcgagaagaaagtggacaggtgcaccagcaacccctgtgccaacgggggacagtgcctgaaccgaggtccaagccgcatgtgccgctgccgtcctggattcacgggcacctactgtgaactccacgtcagcgactgtgcccgtaacccttgcgcccacggtggcacttgccatgacctggagaatgggctcatgtgcacctgccctgccggcttctctggccgacgctgtgaggtgcggacatccatcgatgcctgtgcctcgagtccctgcttcaacagggccacctgctacaccgacctctccacagacacctttgtgtgcaactgcccttatggctttgtgggcagccgctgcgagttccccgtgggcttgccgcccagcttcccctgggtggccgtctcgctgggtgtggggctggcagtgctgctggtactgctgggcatggtggcagtggctgtgcggcagctgcggcttcgacggccggacgacggcagcagggaagccatgaacaacttgtcggacttccagaaggacaacctgattcctgccgcccagcttaaaaacacaaaccagaagaaggagctggaagtggactgtggcctggacaagtccaactgtggcaaacagcaaaaccacacattggactataatctggccccagggcccctggggcgggggaccatgccaggaaagtttccccacagtgacaagagcttaggagagaaggcgccactgcggttacacagtgaaaagccagagtgtcggatatcagcgatatgctcccccagggactccatgtaccagtctgtgtgtttgatatcagaggagaggaatgaatgtgtcattgccacggaggtataaggcaggagcctacctggacatccctgctcagccccgcggctggaccttccttctgcattgtttacattgcatcctggatgggacgtttttcatatgcaacgtgctgctctcaggaggaggagggaatggcaggaaccggacagactgtgaacttgccaagagatgcaatacccttccacacctttgggtgtctgtctggcatcagattggcagctgcaccaaccagaggaacagaagagaagagagatgccactgggcactgccctgccagtagtggccttcagggggctccttccggggctccggcctgttttccagagagagtggcagtagccccatggggcccggagctgctgtggcctccactggcatccgtgtttccaaaagtgcctttggcccaggctccacggcgacagttgggcccaaatcagaaaggagagagggggccaatgagggcagggcctcctgtgggctggaaaaccactgggtgcgtctcttgctggggtttgccctggaggtgaggtgagtgctcgagggaggggagtgctttctgccccatgcctccaactactgtatgcaggcctggctctctggtctaggccctttgggcaagaatgtccgtctacccggcttccaccaccctctggccctgggcttctgtaagcagacaggcagagggcctgcccctcccaccagccaagggtgccaggcctaactggggcactcagggcagtgtgttggaaattccactgagggggaaatcaggtgctgcggccgcctgggccctttcctccctcaagcccatctccacaacctcgagcctgggctctggtccactactgccccagaccaccctcaaagctggtcttcagaaatcaataatatgagtttttattttgtttttttttttttttttgtagtttattttggagtctagtatttcaataatttaagaatcagaagcactgacctttctacattttataacattattttgtatataatgtgtatttataatatgaaacagatgtgtacagga

SEQ ID NO: 10 - protein, 685 aa, Artificial Sequence
  delta-like protein 4 precursor [Homo sapiens] - (DLL4 protein)
MAAASRSASGWALLLLVALWQQRAAGSGVFQLQLQEFINERGVLASGRPCEPGCRTFFRVCLKHFQAVVSPGPCTFGTVSTPVLGTNSFAVRDDSSGGGRNPLQLPFNFTWPGTFSLIIEAWHAPGDDLRPEALPPDALISKIAIQGSLAVGQNWLLDEQTSTLTRLRYSYRVICSDNYYGDNCSRLCKKRNDHFGHYVCQPDGNLSCLPGWTGEYCQQPICLSGCHEQNGYCSKPAECLCRPGWQGRLCNECIPHNGCRHGTCSTPWQCTCDEGWGGLFCDQDLNYCTHHSPCKNGATCSNSGQRSYTCTCRPGYTGVDCELELSECDSNPCRNGGSCKDQEDGYHCLCPPGYYGLHCEHSTLSCADSPCFNGGSCRERNQGANYACECPPNFTGSNCEKKVDRCTSNPCANGGQCLNRGPSRMCRCRPGFTGTYCELHVSDCARNPCAHGGTCHDLENGLMCTCPAGFSGRRCEVRTSIDACASSPCFNRATCYTDLSTDTFVCNCPYGFVGSRCEFPVGLPPSFPWVAVSLGVGLAVLLVLLGMVAVAVRQLRLRRPDDGSREAMNNLSDFQKDNLIPAAQLKNTNQKKELEVDCGLDKSNCGKQQNHTLDYNLAPGPLGRGTMPGKFPHSDKSLGEKAPLRLHSEKPECRISAICSPRDSMYQSVCLISEERNECVIATEV

SEQ ID NO: 11 - DNA, 4163 bp, Artificial Sequence
  Homo sapiens EYA transcriptional coactivator and phosphatase 1 (EYA1), transcript variant EYA1C - (EYA1 nucleotide)
ctcagatgctatctgccgctgctgtttggtggggaaggagcgctgggcgcaaagctgttaccaaacagaacggtgggagctgatggctccgagtttggggcgaggtagaaactctccagtgccacttccgactttaagccttcctgttgccgtccactgtggcgggtttcttcctggggaacacgttttcgctcagtcgctcggcagcccgagcctgcggcagcggccaggcgcctgccccctgcgccgagctttcccctgcagaggcgctccactcccagaagcgccgcggctgcaccagagcgcctgagagcccccgcgcgtacccatccaggagcaaaactatgtcaggaatggaggtttgctaacccagaaaattcgaaggaacacattaaactggtggatgcagcagatgtaagcgctgtgcaaacatctcaagccagttcagatgttgctgtttcctcaagttgcaggtctatggaaatgcaggatctaaccagcccgcatagccgtctgagtggtagtagtgaatcccccagtggccccaaactcggtaactctcatataaatagtaattccatgactcccaatggcaccgaagttaaaacagagccaatgagcagcagtgaaacagcttcaacgacagccgacgggtctttaaacaatttctcaggttcagcaattgggagcagtagtttcagcccacgaccaactcaccagttctctccaccacagatttacccttccaacagaccatacccacatattctccctaccccttcctcacaaactatggctgcatatgggcaaacacagtttaccacaggaatgcaacaagctacagcctatgccacgtacccacagccaggacagccgtacggcatttcctcatatggtgcattgtgggcaggcatcaagactgaaggtggattgtcacagtctcagtcacctggacagacaggatttctcagctatggcacaagcttcagtacccctcaacctggacaggcaccatacagctaccagatgcaaggtagcagttttacaacatcatcaggaatatatacaggaaataattcactcacaaattcctctggatttaatagttcacagcaggactatccgtcttatcccagttttggccagggtcagtacgcacagtattataacagctcaccgtatccagcacattatatgaccagcagcaacaccagcccaacgacaccatccaccaatgccacttaccagcttcaagaaccgccatctggcatcaccagccaagcagttacagatcccacagcagagtacagcacaatccacagcccatcaacacccattaaagattcagattctgatcgattgcgtcgaggttcagatgggaaatcacgtggacggggccgaagaaacaataatccttcacctcccccagattctgatcttgagagagtgttcatctgggacttggatgagacaatcattgttttccactccttgcttactgggtcctacgccaacagatatgggagggatccacccacttcagtttcccttggactgcgaatggaagaaatgattttcaacttggcagacacacatttattttttaatgacttagaagaatgtgaccaagtccatatagatgatgtttcttcagatgataacggacaggacctaagcacatataactttggaacagatggctttcctgctgcagcaaccagtgctaacttatgtttggcaactggtgtacggggcggtgtggactggatgagaaagttggccttccgctacagacgggtaaaagagatctacaacacctacaaaaataatgttggaggtctgcttggtccagctaagagggaagcctggctgcagttgagggccgaaattgaagccctgaccgactcctggttgacactggccctgaaagcactctcgctcattcactcccggacaaactgtgtgaatattttagtaacaactactcagctcatcccagcattggcgaaagtcctgctgtatgggttaggaattgtatttccaatagaaaatatttacagtgcaactaaaataggaaaagaaagctgttttgagagaataattcaaaggtttggaagaaaagtggtgtatgttgttataggagatggtgtagaagaagaacaaggagcaaaaaagcacgcgatgcccttctggaggatctccagccactcggacctcatggccctgcaccatgccttggaactggagtacctgtaacagcgctcggcactttgacagcgcacagctgctctgtgaccagggacagatccagcaggccccagtctcgcatcagcgccggcctccagaacttagcaatttccgcctggtgatgcgcagttgctgtcagtcttgacctctgcctttgtggtgaatggaggaccacgtctatttcatcagaacagctgttgactctagtactgtgaatccagtgaaaataagccatgagaatgttttagcacagcgttatgtgtctgccacattaactacacggttcaaacctgtgaagaaaggacctgcaaacgcttcagttgttagcattttcaatgtgatataaacagcttctccaatacagcaaacctaattgcacaacagagactgaaatgtgtttcctgaataccagtggaggaattttcttgtaaagaaggtttactttttggtgtctcatacccagggtaatctgtacatctctacttatttatgaacagactttttttaaaaagataaaaaaacagctttattgaggtataattcacccaccagacttttttaaacatcaaataattgaagagacaatagcattagaaataagtgattaaaggcctctgcctcacaacatggcaagtacagtactttgaattttagcacattgcatagtagttttaagtatgtctaatttaaacgtataatatgtacatcactgagacaatcatgtacagaaagaatttttggtgtaaatttgtaataatggataattcttttacatattgtttagggaaatgatattgaaaggtagcaatgcctggatagtgaagcatgaggcagcacgtgcacaaattcatgtgccgtgccttatctgagttttcggtataaatatgtagataatggattttttttttttagataatgttgtcaagaccaaaagcatggatgtcaagtgtcagtaaggattttgttttctaaaattttttcctgcatcagttcttctgagggccttgatgaaataacacagcagtttcttaaacaatttgaaacaaaatgagctctcctaccacctcactttttcatttccacactaatgtattatatgtaactacttggaaaaaataattattcaaatgcttcttcccacaaagaatatagatgatagtagatatattttattaataaaatggttcatgaatcggagactaacaaagttttcatgtgctcagaattattaattatcgtgtctgcattttctttcgataaaggaagacacacgatgctaatccggaaatcagcaaactttgcattactccctatgtgcgtattttctctttcttcctgtcaccctgaggaaggttcattgccattgtcatcaccatggaaacaacgttcctctccacctgcattatgtactacatgacaggcatcaatctggggaaataataaaattatcacctttgtcagaccataagagtttctccaaaagtggtcagtttggctgggcaatattttctctcatctaacaaacacaatccattgtcatgaaattacccttaggatgagtcttctttaatcaatcatatattgggcgggaaaaacaccagctttgacccgaagtagttgaagagctacttcattcttttctgaagttgtgtgttgctgctagaaatagtcatttgtgaattatccaaattgtttaaattcacaattgaattagttttttcttcctttttgcttgaagcaaacagttgacaatttttaaccttttcattttatgtttttgtactctgcagactgaaaagacaaagtttatcttggccttactgtataaaggtgtgctgtgtccaccgttgtgtacagaatttttcttcattaattttgtgtttaagttaataaaatttatttgtgatgtactgtaa

SEQ ID NO: 12 - protein, 592 aa, Artificial Sequence
  eyes absent homolog 1 isoform 1 [Homo sapiens] - (EYA1 protein)
MEMQDLTSPHSRLSGSSESPSGPKLGNSHINSNSMTPNGTEVKTEPMSSSETASTTADGSLNNFSGSAIGSSSFSPRPTHQFSPPQIYPSNRPYPHILPTPSSQTMAAYGQTQFTTGMQQATAYATYPQPGQPYGISSYGALWAGIKTEGGLSQSQSPGQTGFLSYGTSFSTPQPGQAPYSYQMQGSSFTTSSGIYTGNNSLTNSSGFNSSQQDYPSYPSFGQGQYAQYYNSSPYPAHYMTSSNTSPTTPSTNATYQLQEPPSGITSQAVTDPTAEYSTIHSPSTPIKDSDSDRLRRGSDGKSRGRGRRNNNPSPPPDSDLERVFIWDLDETIIVFHSLLTGSYANRYGRDPPTSVSLGLRMEEMIFNLADTHLFFNDLEECDQVHIDDVSSDDNGQDLSTYNFGTDGFPAAATSANLCLATGVRGGVDWMRKLAFRYRRVKEIYNTYKNNVGGLLGPAKREAWLQLRAEIEALTDSWLTLALKALSLIHSRTNCVNILVTTTQLIPALAKVLLYGLGIVFPIENIYSATKIGKESCFERIIQRFGRKVVYVVIGDGVEEEQGAKKHAMPFWRISSHSDLMALHHALELEYL

SEQ ID NO: 13 - DNA, 3521 bp, Artificial Sequence
  Homo sapiens forkhead box N1 (FOXN1), transcript variant 1 - (FOXN1 nucleotide)
acagacacaggacggccgagctgacggactgacagacggacagagctcctggccccccagacccgggcccccacgccgacctgcttcactgagcaggccaggactgggtgatggtgtcgctacccccgccgcagtctgacgtcacgctgccgggccccaccagactggagggcgagcgccaaggggacctcatgcaggcaccgggcctcccaggctcccctgccccacagagtaagcatgccggcttcagctgctcgtcatttgtgtccgacggccctccagagaggacaccctcactgcccccacacagcccccgcattgcgtcaccagggcccgagcaagtccagggccactgcccagccggccccggccctgggcccttcaggctctcaccctcagacaagtatcctggctttggctttgaggaggccgcagcaagcagccctgggcgattcctcaagggcagccacgcgcccttccacccgtacaagcggcctttccatgaggacgtcttcccagaggccgagaccaccctggccctcaaaggacactcctttaagaccccagggccgctggaggccttcgaggagatcccagtggacgtggcggaggccgaggccttcctgcctggcttctcagcagaggcctggtgtaacgggctcccctaccccagccaggagcatggcccccaagtcctgggttcagaggtcaaagtcaagcccccagttctggagagtggtgctgggatgttctgctaccagcctcccttgcagcatatgtactgctcctcccagccccccttccaccagtactcgccaggtggtggcagctaccccataccctacctgggctcctcacactatcagtaccagcgaatggcaccccaggccagcaccgatgggcaccagcctctcttcccaaaacccatctattcctacagcatcctcatcttcatggcccttaagaacagtaaaactgggagccttcccgtcagcgagatctacaattttatgacggagcactttccttacttcaagacagcacccgatggctggaagaattctgtccggcacaacctatccctcaacaagtgcttcgagaaggtggagaacaaatcaggaagttcctcccgcaagggctgcctgtgggccctcaatccggccaagatcgacaagatgcaagaggagctgcaaaaatggaagaggaaagatcccattgctgtgcgcaaaagcatggccaagccagaagagctggacagcctcattggagacaagagagaaaagctgggctccccactcctgggctgtccgccccctgggctgtccggctcaggccccatccggcccctggcacccccagctggcctctccccaccactgcactcactccacccagctccaggccccattcctggcaagaaccccctgcaggacctacttatggggcacacaccctcctgctatgggcagacatacttgcacctctcaccaggcctggcccctcctggacccccgcagccattgttcccacagccggacgggcaccttgagctgcgggcccagccaggcaccccccaggactcgcctctgcctgcccacaccccacccagccacagtgccaagctactggccgagccttccccagccaggactatgcacgacaccctgctgccagatggagaccttggcactgacctggatgccatcaatccctcactcactgacttcgacttccagggaaacctgtgggaacagttgaaggatgatagcttggccctcgaccccctggtactggtgacctcatccccgacatcatcttcgatgccaccaccccagccaccacctcactgcttcccccctgggccctgtctgacagagacaggcagtggggcaggtgacttggcagccccgggcagtggtggctccggggcactgggtgacctgcacctcaccaccctctactctgcctttatggagctggagcccacgccccccacggcccctgcaggcccctctgtgtacctcagccccagctccaagcccgtggccctggcatgagctgtgcccagcttcgtcagctccagcgtttgcctggtctggaagtcctggccggccgcccacatcgggctcaccttaaaggtcaaggaaggaaaatactacctgtcccctatgccactaagccaacgtgtgtgtcagctggtagctgggggcgcagaggacatcacctggggtgctgcctctcacacatttctgccacgtggtggcccagctcctcacccagggcccccaaagagcaagcgtctgggcaagaggaaaatgccctgtccctagctcacactcatccacacttaagccctcgtgcacacacacaaattattcagatgtacacccacccacatatcttacagccagaggaaccagcactccatcactgagagcccgacttcgtttctggggcaactgagagctgagcgctttgcttaccaaaagctcagggccctgtgccaggccaaagatccccccagacccccattctgacatccacatgctctgcagtcctggccccctcgtcattttctttcccagaagcgccctgtatttattcccccatcttcatcccaacagcccagcaagaaggaggagacagagagctcctccctgggttgtctgtggacccccccaggagctgctaattggcagcacccactcagccattctctacccatccttagtacatgctctgtccagctttccccagggtgacatacagaaggggcaacatctacccgggcaccaaactgagcccaattccgaagcagtgtttggaaaagcccttgccctcggaaacttgaacatgtcctttttccccagtcccgggtgcaggaagggccccctccaggtcaccacaatctcactcctcctccagaaagaaaagttcacatagtttaggtcctggcttaactctacttatgatggttcaacttacactttttcaactttaagatggtgcaaaagcaatacacattcagtatgttcctcgacttaggatgcggttatgtccgataaacccattgtaaattgaaaccaccgaaagtcaaaaaccactttcgacttacgatgtttcccactcacgatgggtttctccggatgtagccccatcataagttgaggagcacctgttagttacttccaactgtctcaggcaacctgggcattcaggagaaaagcctggcacagggtgacagggaagcacccatgcaaagcacccccgctttgactgcttggcagacctagggtgcccgggaagcacaagggatacccttggccctgcggggtgtggcccaggtgcctgccatgagcctgaccccatcgagggggaacctcagcccaccctgggcctgggggtgacaagaacttccacaggttggccccaggcttataggaggcataagcagatcccgcgatggcttgtcattgtcccctgtcctggaacaataaagcaagtgccttgtggtctcacta

SEQ ID NO: 14 - protein, 648 aa, Artificial Sequence
  forkhead box protein N1 (FOXN1) - (FOXN1 protein)
MVSLPPPQSDVTLPGPTRLEGERQGDLMQAPGLPGSPAPQSKHAGFSCSSFVSDGPPERTPSLPPHSPRIASPGPEQVQGHCPAGPGPGPFRLSPSDKYPGFGFEEAAASSPGRFLKGSHAPFHPYKRPFHEDVFPEAETTLALKGHSFKTPGPLEAFEEIPVDVAEAEAFLPGFSAEAWCNGLPYPSQEHGPQVLGSEVKVKPPVLESGAGMFCYQPPLQHMYCSSQPPFHQYSPGGGSYPIPYLGSSHYQYQRMAPQASTDGHQPLFPKPIYSYSILIFMALKNSKTGSLPVSEIYNFMTEHFPYFKTAPDGWKNSVRHNLSLNKCFEKVENKSGSSSRKGCLWALNPAKIDKMQEELQKWKRKDPIAVRKSMAKPEELDSLIGDKREKLGSPLLGCPPPGLSGSGPIRPLAPPAGLSPPLHSLHPAPGPIPGKNPLQDLLMGHTPSCYGQTYLHLSPGLAPPGPPQPLFPQPDGHLELRAQPGTPQDSPLPAHTPPSHSAKLLAEPSPARTMHDTLLPDGDLGTDLDAINPSLTDFDFQGNLWEQLKDDSLALDPLVLVTSSPTSSSMPPPQPPPHCFPPGPCLTETGSGAGDLAAPGSGGSGALGDLHLTTLYSAFMELEPTPPTAPAGPSVYLSPSSKPVALA

SEQ ID NO: 15 - DNA, 1235 bp, Artificial Sequence
  Homo sapiens major histocompatibility complex, class II, DR alpha (HLA-DRA) - nucleotide
attcttgtctgttctgcctcactcccgagctctactgactcccaacagagcgcccaagaagaaaatggccataagtggagtccctgtgctaggatttttcatcatagctgtgctgatgagcgctcaggaatcatgggctatcaaagaagaacatgtgatcatccaggccgagttctatctgaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacctggaaatcatgacaaagcgctccaactatactccgatcaccaatgtacctccagaggtaactgtgctcacaaacagccctgtggaactgagagagcccaacgtcctcatctgtttcatagacaagttcaccccaccagtggtcaatgtcacgtggcttcgaaatggaaaacctgtcaccacaggagtgtcagagacagtcttcctgcccagggaagaccaccttttccgcaagttccactatctccccttcctgccctcaactgaggacgtttacgactgcagggtggagcactggggcttggatgagcctcttctcaagcactgggagtttgatgctccaagccctctcccagagactacagagaacgtggtgtgtgccctgggcctgactgtgggtctggtgggcatcattattgggaccatcttcatcatcaagggattgcgcaaaagcaatgcagcagaacgcagggggcctctgtaaggcacatggaggtgatggtgtttcttagagagaagatcactgaagaaacttctgctttaatggctttacaaagctggcaatattacaatccttgacctcagtgaaagcagtcatcttcagcattttccagccctatagccaccccaagtgtggatatgcctcttcgattgctccgtactctaacatctagctggcttccctgtctattgccttttcctgtatctattttcctctatttcctatcattttattatcaccatgcaatgcctctggaataaaacatacaggagtctgtctctgctatggaatgccccatggggcatctcttgtgtacttattgtttaaggtttcctcaaactgtgatttttctgaacacaataaactattttgatgatcttgggtggaa

SEQ ID NO: 16 - protein, 254 aa, Artificial Sequence
  HLA class II histocompatibility antigen, DR alpha chain precursor - protein
MAISGVPVLGFFIIAVLMSAQESWAIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTENVVCALGLTVGLVGIIIGTIFIIKGLRKSNAAERRGPL

SEQ ID NO: 17 - DNA, 2437 bp, Artificial Sequence
  Homo sapiens homeobox A3 (HOXA3), transcript variant 1 - (HOXA3 nucleotide)
tctcactagcctcagagcactctcagaagttcagaaactaagaccagaaaagagaagatttttagacagctcatgaaacggtctgcgcggggcggccattggcggcggagtgtcacgtgaccgcgggggcgtgccaatgtgcgccctcacgggtgtcaaacccctgtcagagtgtgcgatcaagatcgtgaaacaacgcgatgcaaaaagcgacctactacgacagctcggcgatctacggtggctacccctaccaggcagccaacgggttcgcttataatgccaatcagcagccgtacccggcgtccgccgctttgggcgccgacggcgagtaccaccgacccgcctgctccctccagtctccctccagcgccgggggccaccccaaggcacacgaactgagtgaggcgtgcctgcgcaccctgagcgccccacctagccagcctccaagcctgggagagccgcccctgcacccgccgccgccccaggccgcgccccctgccccacagccgcctcagcccgcacctcagccccctgcacctacccctgccgcgcccccgcctccctcttctgcctcccctcctcagaatgccagcaacaaccctacccctgccaacgcggccaagagccccctgctcaactcacccacagtggccaaacaaatcttcccctggatgaaagagtctcgacaaaacacaaagcagaaaaccagcagctccagctcaggcgaaagctgcgctggcgacaagagcccgccggggcaggcttcgtccaagcgcgcgcgcacggcctacacgagcgcgcagctggtggagctggagaaagagttccacttcaaccgctacctgtgccggccgcgccgggtggagatggccaatctgctgaacctcactgagcgccagatcaagatctggttccagaatcgccgcatgaagtacaaaaaggatcagaagggcaagggcatgctaacgtcatcggggggccagtctccaagtcgcagccccgtgccccccggagccggtggctatctgaactctatgcattcgctggtcaacagcgtcccgtatgagccccagtcgcccccgcccttctccaagcccccccagggtacctacgggctgccccccgcctcctaccctgcgtccctgcccagctgcgcacccccgccacccccacagaagcgctacacggcggcaggggcgggcgcagggggcacccccgactatgacccgcacgctcatggcctgcagggcaacggcagctatgggaccccacacatacagggaagccccgtcttcgtggggggcagctatgtggagcccatgagcaactccgggccagccctctttggtctaactcacctcccccacgctgcctcgggcgccatggactatgggggtgccgggccgctgggcagcggccaccaccacgggccggggcctggggagccgcaccccacctacacggaccttaccggccaccatccttctcagggaagaattcaggaagcacccaagctcacccacctgtgatagtgggcttggggctacgcgccaggagagtctccccccacccaccttttttctttggttgcttttttttttttttttttttaggttcttcctgccctttccttccttccttttctctcttctccgccccgcactccgtttcccggtttcccccctcgttggtaaggcgtttttatagtttatgtgacgtagcaatcttggttgctggaatggctgtatcatagcgatatttatctcttcctgctcctcgataggccactggccctgcaccctttaccttctccactctttgatcagaaacagggtatatgaacaaattttctagtcgagttttcaatgtgaatttgttcttacattatggctcccgaggggaagcgattactttttttaattttaaattttttttttaattgcacttcttgtaaagagtgagaaaaaaaatcaaaggcgctttgaaacaggggctctctgtgcaaggatgactaagtgtacgtctttccgtgtgtgtatgctggtgaacagtcagatttatttatatttttttgcaagcattgaataatctaagttttaaatattatttatccccatccgttcgtatttatattaaagaattctgtaccctgatggttcagaagggttcttgggccttttgttcaattgtgtattggcgtacttagaattttttttatttgaaagagaaatataattcctttaaacggtaacgatacaataaaaccagagaagatccagcttttgaaaacagtgatttaggtttgtaacatccggcaaaactgaaaaaaaaaatctgtaaacgcgaaaaatactagatttgttttgagagttcttcattccttgctgctcacattctgagaaacaaaaagaaataaagtttttattctgaataa

SEQ ID NO: 18 - protein, 443 aa, Artificial Sequence
  homeobox protein Hox-A3  (HOXA3 protein)
MQKATYYDSSAIYGGYPYQAANGFAYNANQQPYPASAALGADGEYHRPACSLQSPSSAGGHPKAHELSEACLRTLSAPPSQPPSLGEPPLHPPPPQAAPPAPQPPQPAPQPPAPTPAAPPPPSSASPPQNASNNPTPANAAKSPLLNSPTVAKQIFPWMKESRQNTKQKTSSSSSGESCAGDKSPPGQASSKRARTAYTSAQLVELEKEFHFNRYLCRPRRVEMANLLNLTERQIKIWFQNRRMKYKKDQKGKGMLTSSGGQSPSRSPVPPGAGGYLNSMHSLVNSVPYEPQSPPPFSKPPQGTYGLPPASYPASLPSCAPPPPPQKRYTAAGAGAGGTPDYDPHAHGLQGNGSYGTPHIQGSPVFVGGSYVEPMSNSGPALFGLTHLPHAASGAMDYGGAGPLGSGHHHGPGPGEPHPTYTDLTGHHPSQGRIQEAPKLTHL

SEQ ID NO: 19 - DNA, 2238 bp, Artificial Sequence
  Homo sapiens keratin 5 (KRT5) - (KRT5 nucleotide)
aacagagccaccttctgcgtcctgctgagctctgttctctccagcacctcccaacccactagtgcctggttctcttgctccaccaggaacaagccaccatgtctcgccagtcaagtgtgtccttccggagcgggggcagtcgtagcttcagcaccgcctctgccatcaccccgtctgtctcccgcaccagcttcacctccgtgtcccggtccgggggtggcggtggtggtggcttcggcagggtcagccttgcgggtgcttgtggagtgggtggctatggcagccggagcctctacaacctggggggctccaagaggatatccatcagcactagtggtggcagcttcaggaaccggtttggtgctggtgctggaggcggctatggctttggaggtggtgccggtagtggatttggtttcggcggtggagctggtggtggctttgggctcggtggcggagctggctttggaggtggcttcggtggccctggctttcctgtctgccctcctggaggtatccaagaggtcactgtcaaccagagtctcctgactcccctcaacctgcaaatcgaccccagcatccagagggtgaggaccgaggagcgcgagcagatcaagaccctcaacaataagtttgcctccttcatcgacaaggtgcggttcctggagcagcagaacaaggttctggacaccaagtggaccctgctgcaggagcagggcaccaagactgtgaggcagaacctggagccgttgttcgagcagtacatcaacaacctcaggaggcagctggacagcatcgtgggggaacggggccgcctggactcagagctgagaaacatgcaggacctggtggaagacttcaagaacaagtatgaggatgaaatcaacaagcgtaccactgctgagaatgagtttgtgatgctgaagaaggatgtagatgctgcctacatgaacaaggtggagctggaggccaaggttgatgcactgatggatgagattaacttcatgaagatgttctttgatgcggagctgtcccagatgcagacgcatgtctctgacacctcagtggtcctctccatggacaacaaccgcaacctggacctggatagcatcatcgctgaggtcaaggcccagtatgaggagattgccaaccgcagccggacagaagccgagtcctggtatcagaccaagtatgaggagctgcagcagacagctggccggcatggcgatgacctccgcaacaccaagcatgagatctctgagatgaaccggatgatccagaggctgagagccgagattgacaatgtcaagaaacagtgcgccaatctgcagaacgccattgcggatgccgagcagcgtggggagctggccctcaaggatgccaggaacaagctggccgagctggaggaggccctgcagaaggccaagcaggacatggcccggctgctgcgtgagtaccaggagctcatgaacaccaagctggccctggacgtggagatcgccacttaccgcaagctgctggagggcgaggaatgcagactcagtggagaaggagttggaccagtcaacatctctgttgtcacaagcagtgtttcctctggatatggcagtggcagtggctatggcggtggcctcggtggaggtcttggcggcggcctcggtggaggtcttgccggaggtagcagtggaagctactactccagcagcagtgggggtgtcggcctaggtggtgggctcagtgtggggggctctggcttcagtgcaagcagtggccgagggctgggggtgggctttggcagtggcgggggtagcagctccagcgtcaaatttgtctccaccacctcctcctcccggaagagcttcaagagctaagaacctgctgcaagtcactgccttccaagtgcagcaacccagcccatggagattgcctcttctaggcagttgctcaagccatgttttatccttttctggagagtagtctagaccaagccaattgcagaaccacattctttggttcccaggagagccccattcccagcccctggtctcccgtgccgcagttctatattctgcttcaaatcagccttcaggtttcccacagcatggcccctgctgacacgagaacccaaagttttcccaaatctaaatcatcaaaacagaatccccaccccaatcccaaattttgttttggttctaactacctccagaatgtgttcaataaaatgcttttataatataa

SEQ ID NO: 20 - protein, 590 aa, Artificial Sequence
  keratin, type II cytoskeletal 5 [Homo sapiens] - (KRT5 protein)
MSRQSSVSFRSGGSRSFSTASAITPSVSRTSFTSVSRSGGGGGGGFGRVSLAGACGVGGYGSRSLYNLGGSKRISISTSGGSFRNRFGAGAGGGYGFGGGAGSGFGFGGGAGGGFGLGGGAGFGGGFGGPGFPVCPPGGIQEVTVNQSLLTPLNLQIDPSIQRVRTEEREQIKTLNNKFASFIDKVRFLEQQNKVLDTKWTLLQEQGTKTVRQNLEPLFEQYINNLRRQLDSIVGERGRLDSELRNMQDLVEDFKNKYEDEINKRTTAENEFVMLKKDVDAAYMNKVELEAKVDALMDEINFMKMFFDAELSQMQTHVSDTSVVLSMDNNRNLDLDSIIAEVKAQYEEIANRSRTEAESWYQTKYEELQQTAGRHGDDLRNTKHEISEMNRMIQRLRAEIDNVKKQCANLQNAIADAEQRGELALKDARNKLAELEEALQKAKQDMARLLREYQELMNTKLALDVEIATYRKLLEGEECRLSGEGVGPVNISVVTSSVSSGYGSGSGYGGGLGGGLGGGLGGGLAGGSSGSYYSSSSGGVGLGGGLSVGGSGFSASSGRGLGVGFGSGGGSSSSVKFVSTTSSSRKSFKS

SEQ ID NO: 21 - DNA, 1793 bp, Artificial Sequence
  Homo sapiens keratin 8 (KRT8), transcript variant 1 - (KRT8 nucleotide)
attcagcaaatgtttgcggaatgaatggggtgagctggagccaggacctgcaggaagggatctccgcctggttcggcccgcctgcctccactcctgcctctaccatgtccatcagggtgacccagaagtcctacaaggtgtccacctctggcccccgggccttcagcagccgctcctacacgagtgggcccggttcccgcatcagctcctcgagcttctcccgagtgggcagcagcaactttcgcggtggcctgggcggcggctatggtggggccagcggcatgggaggcatcaccgcagttacggtcaaccagagcctgctgagcccccttgtcctggaggtggaccccaacatccaggccgtgcgcacccaggagaaggagcagatcaagaccctcaacaacaagtttgcctccttcatagacaaggtacggttcctggagcagcagaacaagatgctggagaccaagtggagcctcctgcagcagcagaagacggctcgaagcaacatggacaacatgttcgagagctacatcaacaaccttaggcggcagctggagactctgggccaggagaagctgaagctggaggcggagcttggcaacatgcaggggctggtggaggacttcaagaacaagtatgaggatgagatcaataagcgtacagagatggagaacgaatttgtcctcatcaagaaggatgtggatgaagcttacatgaacaaggtagagctggagtctcgcctggaagggctgaccgacgagatcaacttcctcaggcagctatatgaagaggagatccgggagctgcagtcccagatctcggacacatctgtggtgctgtccatggacaacagccgctccctggacatggacagcatcattgctgaggtcaaggcacagtacgaggatattgccaaccgcagccgggctgaggctgagagcatgtaccagatcaagtatgaggagctgcagagcctggctgggaagcacggggatgacctgcggcgcacaaagactgagatctctgagatgaaccggaacatcagccggctccaggctgagattgagggcctcaaaggccagagggcttccctggaggccgccattgcagatgccgagcagcgtggagagctggccattaaggatgccaacgccaagttgtccgagctggaggccgccctgcagcgggccaagcaggacatggcgcggcagctgcgtgagtaccaggagctgatgaacgtcaagctggccctggacatcgagatcgccacctacaggaagctgctggagggcgaggagagccggctggagtctgggatgcagaacatgagtattcatacgaagaccaccagcggctatgcaggtggtctgagctcggcctatgggggcctcacaagccccggcctcagctacagcctgggctccagctttggctctggcgcgggctccagctccttcagccgcaccagctcctccagggccgtggttgtgaagaagatcgagacacgtgatgggaagctggtgtctgagtcctctgacgtcctgcccaagtgaacagctgcggcagcccctcccagcctacccctcctgcgctgccccagagcctgggaaggaggccgctatgcagggtagcactgggaacaggagacccacctgaggctcagccctagccctcagcccacctggggagtttactacctggggaccccccttgcccatgcctccagctacaaaacaattcaattgctttttttttttggtccaaaataaaacctcagctagctctgccaa

SEQ ID NO: 22 - protein, 511 aa, Artificial Sequence
  keratin, type II cytoskeletal 8 isoform 1 [Homo sapiens] - (KRT8 protein)
MNGVSWSQDLQEGISAWFGPPASTPASTMSIRVTQKSYKVSTSGPRAFSSRSYTSGPGSRISSSSFSRVGSSNFRGGLGGGYGGASGMGGITAVTVNQSLLSPLVLEVDPNIQAVRTQEKEQIKTLNNKFASFIDKVRFLEQQNKMLETKWSLLQQQKTARSNMDNMFESYINNLRRQLETLGQEKLKLEAELGNMQGLVEDFKNKYEDEINKRTEMENEFVLIKKDVDEAYMNKVELESRLEGLTDEINFLRQLYEEEIRELQSQISDTSVVLSMDNSRSLDMDSIIAEVKAQYEDIANRSRAEAESMYQIKYEELQSLAGKHGDDLRRTKTEISEMNRNISRLQAEIEGLKGQRASLEAAIADAEQRGELAIKDANAKLSELEAALQRAKQDMARQLREYQELMNVKLALDIEIATYRKLLEGEESRLESGMQNMSIHTKTTSGYAGGLSSAYGGLTSPGLSYSLGSSFGSGAGSSSFSRTSSSRAVVVKKIETRDGKLVSESSDVLPK

SEQ ID NO: 23 - DNA, 5182 bp, Artificial Sequence
  Homo sapiens Nanog homeobox (NANOG), transcript variant 1 - (NANOG nucleotide)
ataaatctagagactccaggattttaacgttctgctggactgagctggttgcctcatgttattatgcaggcaactcactttatcccaatttcttgatacttttccttctggaggtcctatttctctaacatcttccagaaaagtcttaaagctgccttaaccttttttccagtccacctcttaaattttttcctcctcttcctctatactaacatgagtgtggatccagcttgtccccaaagcttgccttgctttgaagcatccgactgtaaagaatcttcacctatgcctgtgatttgtgggcctgaagaaaactatccatccttgcaaatgtcttctgctgagatgcctcacacggagactgtctctcctcttccttcctccatggatctgcttattcaggacagccctgattcttccaccagtcccaaaggcaaacaacccacttctgcagagaagagtgtcgcaaaaaaggaagacaaggtcccggtcaagaaacagaagaccagaactgtgttctcttccacccagctgtgtgtactcaatgatagatttcagagacagaaatacctcagcctccagcagatgcaagaactctccaacatcctgaacctcagctacaaacaggtgaagacctggttccagaaccagagaatgaaatctaagaggtggcagaaaaacaactggccgaagaatagcaatggtgtgacgcagaaggcctcagcacctacctaccccagcctttactcttcctaccaccagggatgcctggtgaacccgactgggaaccttccaatgtggagcaaccagacctggaacaattcaacctggagcaaccagacccagaacatccagtcctggagcaaccactcctggaacactcagacctggtgcacccaatcctggaacaatcaggcctggaacagtcccttctataactgtggagaggaatctctgcagtcctgcatgcagttccagccaaattctcctgccagtgacttggaggctgccttggaagctgctggggaaggccttaatgtaatacagcagaccactaggtattttagtactccacaaaccatggatttattcctaaactactccatgaacatgcaacctgaagacgtgtgaagatgagtgaaactgatattactcaatttcagtctggacactggctgaatccttcctctcccctcctcccatccctcataggatttttcttgtttggaaaccacgtgttctggtttccatgatgcccatccagtcaatctcatggagggtggagtatggttggagcctaatcagcgaggtttctttttttttttttttcctattggatcttcctggagaaaatactttttttttttttttttttgaaacggagtcttgctctgtcgcccaggctggagtgcagtggcgcggtcttggctcactgcaagctccgtctcccgggttcacgccattctcctgcctcagcctcccgagcagctgggactacaggcgcccgccacctcgcccggctaatattttgtatttttagtagagacggggtttcactgtgttagccaggatggtctcgatctcctgaccttgtgatccacccgcctcggcctccctaacagctgggatttacaggcgtgagccaccgcgccctgcctagaaaagacattttaataaccttggctgccgtctctggctatagataagtagatctaatactagtttggatatctttagggtttagaatctaacctcaagaataagaaatacaagtacaaattggtgatgaagatgtattcgtattgtttgggattgggaggctttgcttattttttaaaaactattgaggtaaagggttaagctgtaacatacttaattgatttcttaccgtttttggctctgttttgctatatcccctaatttgttggttgtgctaatctttgtagaaagaggtctcgtatttgctgcatcgtaatgacatgagtactgctttagttggtttaagttcaaatgaatgaaacaactatttttcctttagttgattttaccctgatttcaccgagtgtttcaatgagtaaatatacagcttaaacataaatctttgttaaatttttggttggggtggagaaggaaattagctgaggacactgctatcttagaaatgcatagaaatagctgagcgtggtggcctatgcctgtagtcccagctatttgggaggctgaggcaggagaatcgcttgaacctggtaggtggaggttgtagtgagccaatatcacaccactgcactccagcttgggcgacaagagcaaaactcccatctcaaaaataaataaataaataagtaaataaatgaataataaataaatatgccagttttagaatgaatgaataataaatatgccagttttagaaaaaagggcacttacgtgcattgtacatgtgcatctacaaatgtaatgggttatttccgtgtatgaatctgtaattgatgctaacatttaaagtttcatcagaggcctatatgtaagtaaattctaaggctagttgaaacagatttatgttctcaccttaagagcagctacgcaggcatctgtgaattatactaataatcctttactgtccttttcccatcttgtgcctgccctaaatgtatctgatctgagagcacgtcccttcactctgaagcagcatgatgcaggttatcagagtggttttttccagtcttttctctcattatttccccaattgtccaatgttactaagactatccagtctaattccaagtgttttttttttgttttattttgtttttttttaagactggagtcttactctgttgcccaggctggagtgtagtggagcgatctcggctcaccgcaacctccgcctcccgagttcaagtgattctcctgcctcagcctcccaagtagctgggactacaggggtgagccaccatgcccagctaattgtatttttagtagagacagggtttcactatgttggccaggctggtcttgaactcctgacctcatgatccaccagccttggcctcccaaagtgttgggattacaggcgtgagccactgcgcctggcctatcgtttgtttttttagacagtctctctctattgcccaggctggagtgcagtggcacaatcttggctcactgcaacctccgcctcctgggttcgagcaattctcctgcgtcagcctcctaagtagctgggattacaggcacccaacattacacccggctaatttttggtatttttagtagagatggggtttcaccatgttggccagcctggtctcgaactcctgacctcaggtgatccacctgactcggcctcccaaagtggtgggattataggtgtgagccaccgtgtctgggcaatttttttttttttttttgagatggaacttcacttttgttactcaggctggagcacaatgatgcaatctctactcactgcaacctgcacctcccaggttcaagtgattctcttgcctcagcctttcgagtggctggtattacagggatttgccactatgcccagctaattctttgtatttagtagagatggggtttcaccgtattgaccaggctggtctcgaactcctgacctcaggtaatccaccctcctcggtctcccaaagtgctaggattacaggcatgagccacagtgcccagccttcttttttatttttttaagacgggcttattctgctgctcagactggggcttagtggcaccatttcacctcactgtagcctcgacttcccggattcaagcaatcctcctgcctgagtctctcaaatagcccagactataggtgcacaccaccatgccagctaacttttgtgtttttaggagagacggggtttccccatgttggcctggctagtctcaaactcctgagctcaagcagtctaccagccttggcctcccgaagtgctgagattacaggcatgagtcaccagcctagccctgtattatgttttctgaacttttttttttttaatcacccagtattaatcttcaacctcttggcaggtaagaatttagagatctatttggaataatctttaaagtaacaataataactacagtcaaaagcagtaagattagtgggccaggtgccgtggctcacacctgtaatcccagcacttcgggagaccaaggcaggtggatcacctgaggtcaggagttcaagtttagcatggccaatatggcgaaaccctgtctctactaaaaatacagaaatttgccagaggtcttggtttgcgcctgtaatctcagctacttggaaggcggaggcaggataatcacttgaacccgggaggttgcagtgaccccagataatgccaccccactccagcctgggcgacagagcgtcactgtctcaaaaataaacaaaaataataggagattaggcaaaaaggatagtacaaggctctctgtttaacatttcttcctttctctagaaaattttcctttcttttcccaaaaggaggtgtgtgttacttccaatgctatattttagtagctttaccatattttagtttttaaaattcattattttgatttctcagctttcattttcatttcagtttgtacctgaaggagagaagcatggattgaatttgcctgggttttccgaatttcataatataaatttaaaactcagaagtgggaggaatacaggccttgattagtgaatttcaagctcagttctcggggtgaaggacaaggttaattctactctgagtaattctaccgttcagaaaacctggtgtgtctacaggaaaaaatacaggctgtgactacaaacctaaaagtaaaggcaaacaactgccagacagaggcaggaaagtgggggagagcccttggtaaatattgcatgcctcctggggcccccatgggctttgactcaagggggagatgaagggaatttgttaaagaaagaagtcactgggacagacaaaagcttccggcacaggaagttagttcgactaaggataccgaaatgtaggaatttgaatgggttcctgggtgtgaggttgaagtgaaacttgattgaaaagaagctgctggtggtgagtctctccttaagctattcctggcttctgggtagttggtgtaaactctgtggtccttgaataaagtgtgcttgagaatttta

SEQ ID NO: 24 - protein, 305 aa, Artificial Sequence
  homeobox protein NANOG isoform 1 - (NANOG protein)
MSVDPACPQSLPCFEASDCKESSPMPVICGPEENYPSLQMSSAEMPHTETVSPLPSSMDLLIQDSPDSSTSPKGKQPTSAEKSVAKKEDKVPVKKQKTRTVFSSTQLCVLNDRFQRQKYLSLQQMQELSNILNLSYKQVKTWFQNQRMKSKRWQKNNWPKNSNGVTQKASAPTYPSLYSSYHQGCLVNPTGNLPMWSNQTWNNSTWSNQTQNIQSWSNHSWNTQTWCTQSWNNQAWNSPFYNCGEESLQSCMQFQPNSPASDLEAALEAAGEGLNVIQQTTRYFSTPQTMDLFLNYSMNMQPEDV

SEQ ID NO: 25 - DNA, 1409 bp, Artificial Sequence
  Homo sapiens POU class 5 homeobox 1 (POU5F1), transcript variant 1 (OCT4 nucleotide)
gagtagtcccttcgcaagccctcatttcaccaggcccccggcttggggcgccttccttccccatggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctccgtcaccactctgggctctcccatgcattcaaactgaggtgcctgcccttctaggaatgggggacagggggaggggaggagctagggaaagaaaacctggagtttgtgccagggtttttgggattaagttcttcattcactaaggaaggaattgggaacacaaagggtgggggcaggggagtttggggcaactggttggagggaaggtgaagttcaatgatgctcttgattttaatcccacatcatgtatcacttttttcttaaataaagaagcctgggacacagtagatagacacactta

SEQ ID NO: 26 - protein, 360 aa, Artificial Sequence
  POU domain, class 5, transcription factor 1 isoform 1 (OCT4 - protein)
MAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSN

SEQ ID NO: 27 - DNA, 2512 bp, Artificial Sequence
  Homo sapiens SRY-box transcription factor 2 (SOX2) (SOX2 nucleotide)
gatggttgtctattaacttgttcaaaaaagtatcaggagttgtcaaggcagagaagagagtgtttgcaaaagggggaaagtagtttgctgcctctttaagactaggactgagagaaagaagaggagagagaaagaaagggagagaagtttgagccccaggcttaagcctttccaaaaaataataataacaatcatcggcggcggcaggatcggccagaggaggagggaagcgctttttttgatcctgattccagtttgcctctctctttttttcccccaaattattcttcgcctgattttcctcgcggagccctgcgctcccgacacccccgcccgcctcccctcctcctctccccccgcccgcgggccccccaaagtcccggccgggccgagggtcggcggccgccggcgggccgggcccgcgcacagcgcccgcatgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatgtgagggccggacagcgaactggaggggggagaaattttcaaagaaaaacgagggaaatgggaggggtgcaaaagaggagagtaagaaacagcatggagaaaacccggtacgctcaaaaagaaaaaggaaaaaaaaaaatcccatcacccacagcaaatgacagctgcaaaagagaacaccaatcccatccacactcacgcaaaaaccgcgatgccgacaagaaaacttttatgagagagatcctggacttctttttgggggactatttttgtacagagaaaacctggggagggtggggagggcgggggaatggaccttgtatagatctggaggaaagaaagctacgaaaaactttttaaaagttctagtggtacggtaggagctttgcaggaagtttgcaaaagtctttaccaataatatttagagctagtctccaagcgacgaaaaaaatgttttaatatttgcaagcaacttttgtacagtatttatcgagataaacatggcaatcaaaatgtccattgtttataagctgagaatttgccaatatttttcaaggagaggcttcttgctgaattttgattctgcagctgaaatttaggacagttgcaaacgtgaaaagaagaaaattattcaaatttggacattttaattgtttaaaaattgtacaaaaggaaaaaattagaataagtactggcgaaccatctctgtggtcttgtttaaaaagggcaaaagttttagactgtactaaattttataacttactgttaaaagcaaaaatggccatgcaggttgacaccgttggtaatttataatagcttttgttcgatcccaactttccattttgttcagataaaaaaaaccatgaaattactgtgtttgaaatattttcttatggtttgtaatatttctgtaaatttattgtgatattttaaggttttcccccctttattttccgtagttgtattttaaaagattcggctctgtattatttgaatcagtctgccgagaatccatgtatatatttgaactaatatcatccttataacaggtacattttcaacttaagtttttactccattatgcacagtttgagataaataaatttttgaaatatggacactgaaa

SEQ ID NO: 28 - protein, 317 aa, Artificial Sequence
  transcription factor SOX-2 [Homo sapiens] (SOX-2 protein)
MYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHM

SEQ ID NO: 29 - DNA, 21 bp, Artificial Sequence
  Synthetic Construct - Primer - ACTB Forward
catgtacgttgctatccaggc

SEQ ID NO: 30 - DNA, 21 bp, Artificial Sequence
  Synthetic construct - primer - ACTB Reverse
ctccttaatgtcacgcacgat

SEQ ID NO: 31 - DNA, 21 bp, Artificial Sequence
  synthetic construct - primer - NANOG forward
ccccagcctttactcttccta

SEQ ID NO: 32 - DNA, 22 bp, Artificial Sequence
  synthetic construct - primer - NANOG reverse
ccaggttgaattgttccaggtc

SEQ ID NO: 33 - DNA, 21 bp, Artificial Sequence
  synthetic construct - primer - OCT4 forward
ccgaaagagaaagcgaaccag

SEQ ID NO: 34 - DNA, 21 bp, Artificial Sequence
  synthetic construct - primer - OCT4 reverse
atgtggctgatctgctgcagt

SEQ ID NO: 35 - DNA, 19 bp, Artificial Sequence
  synthetic construct - primer -  SOX2 forward
ccatgaccagctcgcagac

SEQ ID NO: 36 - DNA, 19 bp, Artificial Sequence
  synthetic construct - primer - SOX2 reverse
ggacttgaccaccgaaccc

SEQ ID NO: 37 - DNA, 20 bp, Artificial Sequence
  synthetic construct - sgRNA
cacagctcatgccagggcca

SEQ ID NO: 38 - DNA, 20 bp, Artificial Sequence
  synthetic construct - sgRNA
gctgggcacagctcatgcca

SEQ ID NO: 39 - DNA, 24 bp, Artificial Sequence
  synthetic construct - primer - 1L
aatctaccttccttgggagactgg

SEQ ID NO: 40 - DNA, 20 bp, Artificial Sequence
  synthetic construct - primer - 2R
taaacggccacaagttcagc

SEQ ID NO: 41 - DNA, 20 bp, Artificial Sequence
  synthetic construct -  primer - 3R
cctctcacacatttctgcca